(19) United States
(12) Patent Application Publication
(10) Pub. No.: US 2025/0257354 A1
(43) Pub. Date: Aug. 14, 2025

(54) METHODS AND COMPOSITIONS RELATED TO TRNA THERAPEUTICS FOR TREATING VISION LOSS

(71) Applicants: UNIVERSITY OF IOWA RESEARCH FOUNDATION, Iowa City, IA (US); WISCONSIN ALUMNI RESEARCH FOUNDATION, Madison, WI (US)

(72) Inventors: Christopher Ahern, Iowa City, IA (US); Bikash Pattnaik, Madison, WI (US); Shaoqin Gong, Madison, WI (US); Yuyuan Wang, Madison, WI (US)

(73) Assignees: UNIVERSITY OF IOWA RESEARCH FOUNDATION, Iowa City, IA (US); WISCONSIN ALUMNI RESEARCH FOUNDATION, Madison, WI (US)

(21) Appl. No.: 18/857,516

(22) PCT Filed: Apr. 21, 2023
(86) PCT No.: PCT/US2023/019454

§ 371 Date: Oct. 17, 2024

Related U.S. Application Data
(60) Provisional application No. 63/333,442, filed on Apr. 21, 2022.

(51) Int. Cl. C12N 15/113; A01K 67/0275; A61K 9/00; A61K 9/51; A61K 48/00; C12N 15/86

(52) U.S. Cl. CPC C12N 15/113; A01K 67/0275; A61K 9/0051; A61K 9/51; A61K 48/005; C12N 15/86; A01K 2217/072; A01K 2227/105; A01K 2267/0306; C12N 2320/32; C12N 2320/34; C12N 2750/14143

(57) ABSTRACT

Provided herein are compositions and methods related to tRNA therapeutics for treating vision loss and blindness.

### RELATED APPLICATIONS

This application claims the benefit of priority under 35 U.S.C. § 119 (e) of U.S. Provisional Application Ser. No. 63/333,442, filed on Apr. 21, 2022, the entire contents of which are incorporated herein by reference.

### STATEMENT REGARDING FEDERALLY SPONSORED RESEARCH

This invention was made with government support under EY032434 awarded by the National Institutes of Health. The government has certain rights in the invention.

### FIELD OF THE INVENTION

Provided herein are compositions and methods related to tRNA therapeutics for treating vision loss and blindness.

### SUMMARY OF THE INVENTION

Premature termination codons (PTCs) arise from single nucleotide mutations that convert a canonical triplet nucleotide codon into one of three stop codons, e.g., TAG, TGA, or TAA. One such example, Leber congenital amaurosis (LCA), a rare type of inherited eye disorder that causes severe vision loss at birth that affects about 200,000 individuals and stems from a variety of gene defects. Of this, PTC-related eye diseases account for about 22,000 individuals (considering an incidence of ˜11%), and this number is likely to grow with expanded access to sequencing analysis. The FDA has approved AAV-mediated gene therapy, Luxturna®, only for biallelic RPE65 (mutation-associated retinal dystrophy), yet patients benefiting from Luxturna represent a fraction of the LCA mutations (47).

Eye and retina, in particular, are attractive tissues as a drug target because of its anatomic and immunological advantages, as demonstrated by anti-VEGF delivery through intravitreal routes (49) and RPE65 gene therapy through subretinal routes (50). Systemic drug delivery to the retina is challenging due to the blood-retina barrier that restricts adequate drug availability in the retina. Intravitreal administration has been deemed the most favored as multiple dosing intervals are tolerated (51) except for targeting to the posterior retina, which poses diffusion limitations. Drug delivery to the posterior retina is achievable through sub-retina and suprachoroidal routes (52, 53). Importantly, there are multiple methods for viral and non-viral delivery of nucleic acid-based therapies for the eye; however, until recently, there have been limited options for a generalizable in vivo “deliverable”. Provided herein is an approach to repair one or more PTCs across one or more genes. The approach provided can be used to treat vision loss and blindness. Thus, provided herein are methods and compositions for doing so.

In one aspect, a method of reading through one or more premature termination codons (PTCs) in cells of the eye, comprising delivering one or more of the tRNAs, one or more of the oligonucleotide(s) encoding the tRNA(s), one or more of the vector(s) encoding the tRNA(s), one or more of the expression cassette(s) encoding the tRNA(s), or one or more of the composition(s) comprising any of the foregoing provided herein to cells of the eye in an amount effective to read through the one or more PTCs is provided.

In another aspect, a method of restoring protein function in cells of the eye, comprising delivering one or more of the tRNAs, one or more of the oligonucleotide(s) encoding the tRNA(s), one or more of the vector(s) encoding the tRNA(s), one or more of the expression cassette(s) encoding the tRNA(s), or one or more of the composition(s) comprising any of the foregoing provided herein to cells of the eye in an amount effective to restore protein function in the cells is provided.

In another aspect, a method of restoring vision in a subject, comprising administering one or more of the tRNAs, one or more of the oligonucleotide(s) encoding the tRNA(s), one or more of the vector(s) encoding the tRNA(s), one or more of the expression cassette(s) encoding the tRNA(s), or one or more of the composition(s) comprising any of the foregoing provided herein to cells of the eye in an amount effective to restore vision in the subject is provided.

In one embodiment of any one of the methods provided herein, the composition for use is any one of the compositions provided herein.

### BRIEF DESCRIPTION OF THE FIGURES

FIG. 1 shows the folded tRNA structure (Left) with the anticodon region shown as a loop at the bottom of the structure. In this example, UCG is the wildtype tRNA anticodon for decoding the CGA Arginine codon. Right, mutation of UCG-to-UCA within the anticodon (underline) converts this tRNA to a UGA (RNA) stop codon suppressor.

FIG. 2 shows tRNA suppressors for every known human disease-causing PTC identified.

FIG. 3 shows engineered tRNA do not suppress normal protein termination codons aka “real stops”. The average log 2-fold change of normalized ribosome footprint occupancy was plotted for each nucleotide from −50 to +50 nt surrounding stop codons of transcriptome (18,101 sequences). The cartoon illustrates the ˜15 nt offset from the 5′ end of ribosome footprint to the first base position of stop codon in the ribosome. Right, inset, shows ribosome footprint densities on 3′UTRs are plotted as log 2-fold change for reads of treated cells versus control (puc57GG emptyvector). Transcripts were grouped by their endogenous UAA, UAG, and UGA stop codons. Each point represents the mean of two replicates for a transcript. Error bars show Mean±SD of the log 2-fold changes.

FIGS. 4A-4D show high-fidelity post-transcriptional PTC repair with ace-tRNA. FIG. 4A shows high-fidelity encoding, as determined by mass spectrometry, of PTC repaired peptides in HEK cells for Glycine^{UGA} and Tryptophan^{UGA} tRNA. FIG. 4B show ace-tRNA (for Gly^{TGA}) display stop codon specificity and out-perform the read-through compound gentamicin (100 μM) and G418 (10 μM). FIG. 4C shows missense mutation W53Q and W53E potassium channels. W53Q and W53E channels display reduced expression and altered gating. FIG. 4D shows the average plot of membrane potential showing depolarization for W53E and W53Q channels.

FIG. 5 demonstrates positionally independent rescue of PTC. Center, topology of the 24 transmembrane, 250 kD, Nav1.2 sodium channel encoded by the SNC2A gene, a channel with similar topology to CACNA1F, where PTC cause night blindness. Upper left shows a representative family of wild-type voltage-gated sodium currents elicited by 20 millisecond depolarizations from a hold potential of −140 mV expressed in HEK cells. Five PTC sites are shown that are associated with neurological disease (developmental delay): R607X, S700X, G1013X, R1626X and W1716X from (ClinVar). Each mutant shows no measurable activity when expressed alone. For each site, exemplary rescued sodium currents are shown for each condition where the mutant SCN2A cDNA was co-transfected.

FIGS. 6A-6C show a schematic illustration of the SNPs designed for intravitreal injections. FIG. 6A shows the design of the intravitreally injected SNPs for PR-/RPE-targeting delivery. FIG. 6B shows a flowchart for the microemulsion process to prepare SNPs. FIG. 6C shows a schematic diagram of the cellular uptake of the SNPs and the subcellular release of payloads into the cytosol.

FIGS. 7A-7D demonstrate delivery efficiency of nucleic acids (pDNA and mRNA) and CRISPR RNP by SNPs. Transfection efficiency of the (FIG. 7A) pDNA- and (FIG. 7B) mRNA-loaded SNPs in HEK 293T cells. (FIG. 7C) Genome-editing efficiency of RNP-loaded SNPs in GFP-expressing HEK 293T cells. (FIG. 7D) Viability of HEK 293T cells treated with Lipo 2000 and SNPs with different concentrations.

FIGS. 8A-8E show Cre mRNA- or Cas9 RNP-loaded SNPs induced genome editing in vivo in Ai14 mice via subretinal injection. (FIG. 8A) The tdTomato locus in the Ai14 reporter mouse. A LoxP-flanked stop cassette containing 3 Ai14 sgRNA target sites prevents downstream tdTomato expression. Cre-Lox recombination or Cas9 RNP guided excision of the stop cassette results in tdTomato expression. (FIGS. 8B-8D) Representative images of tdTomato+ cells 4 days post-injection of (FIG. 8B) Cre mRNA-loaded SNP-11cRal editing PRs, (FIG. 8C) Cre mRNA-loaded SNP-ATRA editing RPE, (FIG. 8D) PBS as the control. (FIG. 8E) Representative images of tdTomato+ cells 12 days post-injection of Cas9 RNP-loaded SNP-ATRA. Scale bars: 500 μm in RPE/retina floret images; 100 μm in zoom-in images.

FIGS. 9A-9B shows 5X tRNA-loaded SNPs induced transfections in vivo in Ai14 mice via subretinal injection. (FIGS. 9A-9B) Representative images of tRNA+ cells (GFP+) 4 days post-injection of (FIG. 9A) PBS as the control, and (FIG. 9B) 5×tRNA-loaded SNP-ATRA targeting RPE cells.

FIGS. 10A-10D show RNP+ssODN-loaded SNP-CPP uptake by iPSC-RPE after 4 days of treatment. Control cells at 20× and 50× (FIG. 10A) bright field images. (FIG. 10B) ATTO-488 fluorescence after delivery of 3 μg of RNP in a superimposed image and reconstituted z-stack fluorescence image (lower panel). (FIG. 10C) Same as B but ATTO-488 positive cells after delivery of 6 ug of RNP. (FIG. 10D) Same as in B and C, ATTO-488 positive cells after delivery of 12 ug of RNP.

FIGS. 11A-11I demonstrate putative Kir7.1 loss-of-function cure through gene augmentation. (FIG. 11A) LCA16 patient derived iPSC-RPE, and TAGstop codon (FIG. 11B). (FIG. 11C) Plot of the average current-voltage (I/V) curve for Kir7.1 currents from patient iPSC-RPE vs. controliPSC-RPE. (FIG. 11D) Control iPSC-RPE and TGG genotype (FIG. 11E). (FIG. 11F) I-V plot as in F showing depolarization of membrane potential. (FIG. 11G) I-V plot using K+ or Rb+ in LCA16 hiPSC-RPE cells after gene therapy. (FIG. 11H) An average membrane potential plot for control, LCA16 and after gene therapy. (FIG. 11I) Distribution of wildtype Kir7.1 channel in LCA16 iPSC-RPE after gene therapy.

FIGS. 12A-12F demonstrates Kir7.1 LCA16 mutant functional recovery using tRNA Trp (FIG. 12A) W53X cells showing cytoplasmic distribution of GFP. (FIG. 12B) Membrane localization of tRNA^{Trp} treated Kir7.1 channel (FIG. 12C) membrane potential, (FIG. 12D) Normal Kir7.1 current, (FIGS. 12E, 12F). increased Rb permeability after treatment.

FIGS. 13A-13B (from Capowski et al., 2019) shows light microscopic (LM) identification of distinct morphological stages in live cultures of differentiating hPSC-derived 3D retinal organoids. (FIG. 13A) Schematic of the hPSC retinal differentiation protocol that can be used. (FIG. 13B) Timing and overlap of morphological stages observed across all 16 lines tested. Representative live LM phase images of stage 1, stage 2, and stage 3 organoids. Scale bars=100 microns. Stage 3 right panel is a magnification of the box outlined in Stage 3 light panel showing outer segments, and right-most panel is a multi-lobulated stage 3 organoid. Scale bar=50 microns.

FIGS. 14A-14L show stage 3 organoids possess a well-defined outer nuclear-like layer (ONL) with maturing rods and cones. (FIG. 14A) ICC image of a stage 3 organoid (d200) demonstrates an ONL region containing an outermost layer of M/L− OPSIN+/CRX+cones with M/L− OPSIN+surfaceprojections (arrow), and an underlying 3-5 nuclei-thick layer of NRL+/CRX+ rods. Mis-localized CRX+ PRs, some co-expressing M/L OPSIN or NRL, are also present below the ONL (asterisk) (scale bar=100 microns). (FIGS. 14B-14C) Higher magnification ICC images of stage 3 organoids (d160) showing the location and morphology of M/L−OPSIN+ (FIG. 14B) and S−OPSIN+ (FIG. 14C) cones and NR2E3+ rods in the ONL (B′ and C′, merge in B″ and C″) (scale bars=25 microns). (FIGS. 14D, 14E) NRL+ rod (FIG. 14D) and ARR3+ cones (FIG. 14E) % within the ONL derived from 3 different WT hiPSC lines. (FIGS. 14F, 14G) M/L−OPSIN+ (FIG. 14F) and S−OPSIN+ (FIG. 14G) cones % within the total ARR3+ cone population. (FIG. 14H) 3D multiphoton rendering of a 259d old organoid showing the surface distribution of RHO+ rods and M/L−OPSIN+ cones (scale bar=100 microns). (FIGS. 14I-14K) Confocal images of a single z plane through the organoid shown in panel H immunostained for M/L OPSIN (FIG. 14I) and RHO (FIG. 14J; merge in FIG. 14K). (FIG. 14L) Light microscopic image of the organoid taken prior to fixation showing a lobulated region of pigmented RPE (asterisk in FIGS. 14I-14L) and hair-like projections (arrow) covering the remainder of the organoid surface. Scale bar for FIGS. 14I-14L=500 microns.

FIGS. 15A-15G show transduction of photoreceptors in hiPSC-derived retinal organoids using two different AAV capsids. CAG-GFP expression constructs were packaged in AAV5 or AAV7m8 and used to transduce retina organoids at d90 of differentiation. Expression of GFP two weeks post-transduction is shown in whole organoids (no infection control: FIG. 15A (left panel), FIG. 15A (right panel); AAV5: FIG. 15B (left panel), FIG. 15B (right panel); AAV7m8: FIG. 15C (left panel), FIG. 15C (right panel) and in sections (AAV5: FIG. 15D-15F; AAV7m8: FIG. 15G). Overall, AAV5 was more efficient than AAV7m8; however, improvement is likely with both capsids following further titer optimization. Scale bars: FIG. 15A-15C=200 μm; FIG. 15D-15F=50 μm; FIG. 15G=25 μm.

FIGS. 16A-16I demonstrate AAV2.7m8 mediated transduction of iPSC-RPE and mouse RPE in vivo. (FIG. 16A) Small tRNA size enables multiple tRNA copies for multiplexed payloads and a reporter within a 4 kb. (FIG. 16B) Reporters for nanoluciferase-stop and eGFP-stop will be used with the corresponding stop codon (TGA, TAA, TAG) in the reporter construct. (FIG. 16C) Delivery and suppression activity with AAV (2/2) of Nluc-TGA+3×Arg-UGA suppressor in day 14 lentivirus-mediated NGN2 expression induced differentiation of iPSCs into excitatory neurons. X-axis, 1 μl or 0.1 μl (Titer vg/ml 2.2E13) per well (96-well plate). (FIG. 16D, left panel) Bright field image of hiPSC-RPE and (FIG. 16D, right panel) GFP expression after transduction. The same rAAV2.7m8 vector was delivered to the mouse eye by intravitreal injection (FIG. 16E). (FIG. 16F) Fundus auto fluorescence image of mouse RPE after transduction. (FIG. 16G) OCT image of intact retinal layers. (FIG. 16H, left panel) Flat mount of RPE cells imaged for GFP expression. (FIG. 16H, right panel) Higher magnification of GFP expressing RPE cells as in (FIG. 16H, left panel).

FIGS. 17A-17C show a design of the SNPs for subretinal injections. (FIG. 17A) A schematic illustration of SNPs, the injection method and the penetration of the retinal barriers by SNPs. (FIG. 17B) A schematic flowchart for fabricating the SNPs and the surface modification with HA coatings. (FIG. 17C) Synthesis schemes for HA conjugated with ligands (11cRal and ATRA) used for SNP surface coating.

FIGS. 18A-18C shows a design for subretinal injected SNPs as a potential alternative approach. (FIG. 18A) A schematic illustration of SNPs and the injection method. (FIG. 18B) A schematic flowchart for fabricating the SNPs and the surface modifications. (FIG. 18C) Synthesis schemes for SNPs conjugated with ligands (e.g., 11cRal, ATRA and CPP (e.g., TAT or POD)).

FIG. 19 shows a schematic illustration of AAV/SNP/MC mediated targeting of either RPE or PR to deliver therapeutic tRNA.

FIGS. 20A-20F demonstrate ace-tRNA delivery through minicircle and viral means.

FIG. 21 provides a schematic illustration of fabrication of HA-SNP.

FIG. 22 shows nucleic acid and RNP delivery efficiency of SNP in Ai14 mice via subretinal injection. Efficient delivery of Cre-mRNA by SNP-PEG-ATRA in mouse RPE. D1, RPE floret of eyes subretinally injected with Cre-mRNA-encapsulated SNP; D2, 20× magnification images of tdTomato+ RPE tissue; D3, RPE floret of PBS controls.

FIGS. 23A-23E demonstrate altered modulation of Kv2.1 current by Kv8.2 E148*. (FIGS. 23A-23C) current traces to voltage pulses. (FIG. 23D) Plot of maximum current amplitude. (FIG. 23E) Activation curve color coding as in FIG. 23D.

FIG. 24 shows nuclear localization of ATTO 550 in the 3D organoid PRs.

FIG. 25 shows PCR products and Sanger sequencing results.

### DETAILED DESCRIPTION OF THE INVENTION

There are limited therapeutic options for the repair of Premature Termination Codons (PTCs). Nonsense mutations can arise from the insertion of an inherited or spontaneous nucleotide alternation within a single codon to introduce an in-frame PTC within the mRNA sequence (1). Ten amino acid codons are vulnerable to PTC conversion by a single nucleotide substitution: arginine, tryptophan, tyrosine, cysteine, glutamic acid, lysine, glutamine, serine, leucine, and glycine (2, 3). This is due to the fact that one of their triplet codons is within one-base of one of the three stop codons (TGA, TAA, TAG). Because these mutations terminate protein synthesis, they are phenotypically severe, more pathogenic, yet account for 10-15% of all human genetic diseases (4). Taken together, “PTC diseases” represent a constellation of hundreds of PTC-containing genes, resulting in a multitude of individual rare diseases that share a common molecular basis: the introduced termination codon. Each afflicted gene itself may have hundreds of unique PTC genetic lesions, which, taken together, give rise to a therapeutic challenge of epic proportions. Recent estimates suggest as many as 2.5 to 3 million patients in the US alone suffer from a PTC disease, either from spontaneous or inherited mutations. There are limited clinical or therapeutic options for PTC repair. Small molecules, such as aminoglycosides (5), dipeptides (6), and oxadiazoles (7), promote “read-through” at PTC (8) but results in the encoding of a near-cognate amino acid (9), effectively generating a missense mutation in place of the PTC. Ion channels that are essential to visual transduction, such as bestrophin, Kir7.1, Kv8.2, TRPM1, Ca2+-channels, are poorly tolerant of missense mutations, which can cause gain-of-function or loss-of-function neurological disorders (10, 11). Furthermore, aminoglycosides, which are the choice read-through agents, are oto- and nephrotoxic when used chronically (12), and the first-in-class read-through compounds such as oxadiazole and Ataluren have displayed unexpectedly low efficacy in patient populations (ACT DMD Phase 3 clinical trial, NCT01826487; ACT CF, NCT02139306).

Recent and ongoing advances in CRISPR/Cas9-mediated genome editing provide a potentially permanent solution for diseases resulting from nonsense mutations, and such approaches have found recent success in the repair of retinal genes (13). However, aspects of this technology impart hurdles for its rapid therapeutic use (14, 15), including cell type-specific delivery, the efficiency of homologous recombination, and the frequency of off-target editing. It is also a complicated process of CRISPR/Cas9 construct validation and off-target assessment for each of the hundreds of contextually unique PTCs causing blindness. AAV delivery is promising, but at 5-9 kb per gene transcript, ion channel genes largely exceed the AAV payload capacity limit, thus frustrating the utility of rapidly advancing AAV therapies for tissue-specific gene replacement. Additionally, when gene delivery is successful, the potential complications arising from AAV-mediated gene augmentation are non-trivial and include accidental gene over- or under-expression as well as an escape from regulatory pathways which fine-tune ion channel expression in precision detection systems, such as those in vision. Therefore, there are limited clinical options for the many ion channel PTCs, and the therapeutic pipeline is thin.

A universal therapy method based on anti-codon edited short transfer RNA (ace-tRNA) to repair retinal PTCs in vivo (16) has been developed. Data show that this approach displays the versatility of a small molecule therapeutic and the precision of gene editing, with essentially no interactions with ‘real’ stop codons. Notable for this approach is its agnostic nature, that is, an ace-tRNA formulation for the repair of an Arg->TGA mutation can be used to treat any such gene carrying an Arg->TGA mutation at any position within the afflicted gene. Furthermore, new ace-tRNA formulations for the repair of every known PTC in retinal disease have been identified, thus providing a single, sweeping approach for treating all nonsense-mediated inherited retinal disorders. After achieving stable delivery to retinal tissue, the ace-tRNA technology can provide a new form of long-lasting gene therapy for multiple inherited blinding disorders. Provided herein is an ace-tRNA technology as a retinal therapy for the treatment of genetic ocular diseases caused by premature termination codons.

Provided herein are compositions and methods for the repair of PTC(s) in retinal genes with tRNA therapeutics. Multiple gene mutations account for inherited retinopathy, including retinitis pigmentosa, cone-rod dystrophy, Leber congenital amaurosis (LCA), macular dystrophy, and degeneration (17, 18). The subjects that can benefit from the compositions and methods provided may have any disease or disorder that result in vision loss or blindness, such as any one of the diseases or disorders provided herein. Nonsense mutations cause ˜15% of genetically inherited human disease, which holds for retinopathies. Still, for a given gene, this percentage can be much higher, upwards of ˜40% in some cases (HGMD 2019.2). The first mechanistic step in the act of “seeing” involves phototransduction by the PR and their maintenance by RPE (19). Nonsense mutations in the following genes in both RPE and PR account for retinopathies, and all of these are tRNA targets. Thus, in one embodiment, any one of the methods or compositions provided herein can be directed to one or more PTCs in one or more of these genes. A subject may be any subject in need of vision restoration and may be any subject with a PTC mutation in one or more retinal genes, such as those provided herein, such as below. Genes and PTCs that can be repaired in vitro and in vivo are shown, and still others are known in the art. Far left, disease types, OMIM listing, gene names and mutation at the DNA and RNA level. Candidate tRNA types for each amino acid and mutation have been identified (Table 1), and have shown limited contact with real stop codon for each major tRNA family type. In any one of the compositions or methods provided herein, the tRNA for us is any one of these tRNA types for the amino acid and mutation identified. In any one of the compositions or method provided herein, such tRNA is for use for the relevant disease or disorder as provided herein or as otherwise known in the art.

TABLE 1

| PTC Blindness | OMIM | Gene | DNA | RNA | Ref. |
|---|---|---|---|---|---|
| VMD | 607854 | BEST1, TYR29TER | 87C-G | UAC-UAG | (20) |
| VMD | 611809 | BEST1, ARG200TER | 598C-T | CGA-UGA | (21) |
| VMD | 611809 | BEST1, SER517TER | 1550C-G | UCA-UGA | (22) |
| Cone Dystrophy | 610356 | KCNV2, GLU143TER | 427G-T | GAA-UAA | (23) |
| Cone Dystrophy | 610356 | KCNV2, GLU306TER | 916G-T | GAG-UAG | (23) |
| Cone Dystrophy | 610356 | KCNV2, GLN76TER | 226C-T | CAA-UAA | (24) |
| Cone Dystrophy | 610356 | KCNV2, GLU148TER | 442G-T | GAA-UAA | (24) |
| Cone Dystrophy | 610478 | CACNA2D4, TYR802TER | 2406C-A | UAC-UAA | (25) |
| Cone Dystrophy | 610478 | CACNA2D4, ARG628TER | 1882C-T | CGA-UGA | (26) |
| CSNB2A | 300071 | CACNA1F, ARG958TER | 2172C-T | CGA-UGA | (27) |
| CSNB2A | 300071 | CACNA1F, ARG830TER | 2488C-T | CGA-UGA | (28) |
| LCA16 | 614186 | KCNJ13, TRP53TER | 158G-A | UGG-UAG | (29) |
| LCA16 | 614186 | KCNJ13, ARG166TER | 496C-T | CGA-UGA | (30) |
| CSNB1C | 613216 | TRPM1, GLN11TER | 31C-T | CAA-UAA | (31) |
| CSNB | 613216 | TRPM1, LYS294TER | 880A-T | AAG-UAG | (32) |
| CSNB | 613216 | TRPM1, ARG877TER | 2629C-T | CGA-UGA | (32) |
| CSNB | 613216 | TRPM1, SER882TER | 2645C-G | UCA-UGA | (33) |

As an example, KCNJ13 codes for Kir7.1, a homotetrameric inwardly rectifying potassium channel that localizes to the apical membrane of RPE (34). This channel protein is necessary for the maintenance of potassium ion homeostasis in the subretinal space, and its function is essential for vision (35). Sergouniotis et al. identified an R166X mutation in a Middle Eastern family in which a C to T mutation changed arginine (CGA) to a premature stop codon (TGA) (30). The R166X mutant lacked the C-terminus tail, which is necessary for homotetramer formation and localization to the membrane, thus resulting in strongly reduced channel activity. A 12 yr old proband of Middle Eastern origin homozygous for a nonsense mutation 158G>A, resulting in W53X PTC, has been studied. Clinical presentations were characteristic LCA phenotype, including an abnormal ERG, nystagmus, RPE pigmentation, and loss of outer retinal structures. At six years of age, ERGs a- and b-wave amplitudes were reduced. At nine years of age, pattern electroretinogram was undetectable, indicative of loss of PR function (29, 30, 36-38).

As another example, the KCNV2 gene is located on chromosome 9p24.2 (39). A rare form of cone dystrophy with enhanced rod electroretinogram is caused by homozygous nonsense mutations in the KCNV2 gene that encodes for Kv8.2 protein (40). Kv8.2 makes a heterotetrameric complex with Kv2.1 to form a unique photoreceptor outward rectifying potassium channel responsible for circulating dark current. Wu et al. first showed a nonsense mutation within exon 1 of the KCNV2 gene, 427G-T resulting in mutation E143X. Within the exon 1, Wissinger et al. found several nonsense mutations (E73X, Q76X, E148X, K260X, and Q287X) by sequencing the whole coding sequence and 442G-T transition that contributed to E148X was found in several patients tested (24). Another homozygous 916G-T mutation caused E306X to stop codon (23).

As a further example, Bestrophin 1 (BEST1 or VMD2) is on chromosome 11 (11q12.3) and encodes a transmembrane 585 amino acid chloride channel that localizes to the basal membrane of the RPE (41, 42). In the RPE, Bestrophin is essential for the regulation of fluid and ion homeostasis and ion transport (42). BEST1 mutations cause both autosomal dominant (Best vitelliform macular dystrophy, BVMD) and autosomal recessive bestrophinopathy (ARB). Burgess et al. identified a nonsense mutation in BEST1 that causes BVMD (R200X mutation resulting from a 598C>T substitution (21)), and Schatz et al. have described the Y29X nonsense mutation (20). Recently, Chibani et al. found a nonsense mutation C1550G (p.S517X) in a Tunisian BVMD family (22). However, VMD is a rare disorder with an unknown incidence.

TRPM1 gene is mapped to chromosome 15q13-q14 that encodes a cation permeable transient receptor ion channel. TRPM1 channels are present in the ON-bipolar cells in the retina, which controls the mGluR6 transduction cascade. In a recent cohort study, autosomal recessive inheritance was found to be 1:6210 within the Jewish community (32). Several nonsense mutations exist in a compound heterozygous state, including Q11X, S882X, and Y1035X (31, 33). CACNA2D4 is present on chromosome 12p13.3 and encodes a voltage-dependent calcium channel alpha-2/delta subunit 4 (43). In the PR terminals, these specialized calcium channels mediate calcium influx to initiate glutamate release (25). By whole-genome sequencing, two probands with homozygous nonsense mutation R628X have presented nonprogressive cone dysfunction (26). Similarly, two siblings with a slowly progressive cone dysfunction were identified to carry Y802X homozygous nonsense mutation (26).

As a further example, CACNA1F is present on Xp11.23 and encodes a 1,985-aminoacid L-type calcium channel protein present in the outer nuclear layer, inner nuclear layer, and ganglion cell layer (44-46). Two of the identified nonsense mutations identified in congenital stationary night blindness patients are R830X and R958X (27, 46).

Still others will be apparent to those of ordinary skill in the art. Increased genetic testing has enabled the discovery of causative gene mutations for channelopathies of the outer retina that cause blindness. Despite the established physiological relevance of these mutations, the diverse clinical manifestations have challenged disease management. Currently, there are no FDA approved treatment options, which is compounded by the complexities of these membrane proteins. Further, while specific disorders are rare with genetic heterogeneity, they share a common genetic lesion (the premature termination codon). Thus, the methods and compositions provided herein, and related to tRNA therapeutics, can provide treatment that has until now been elusive.

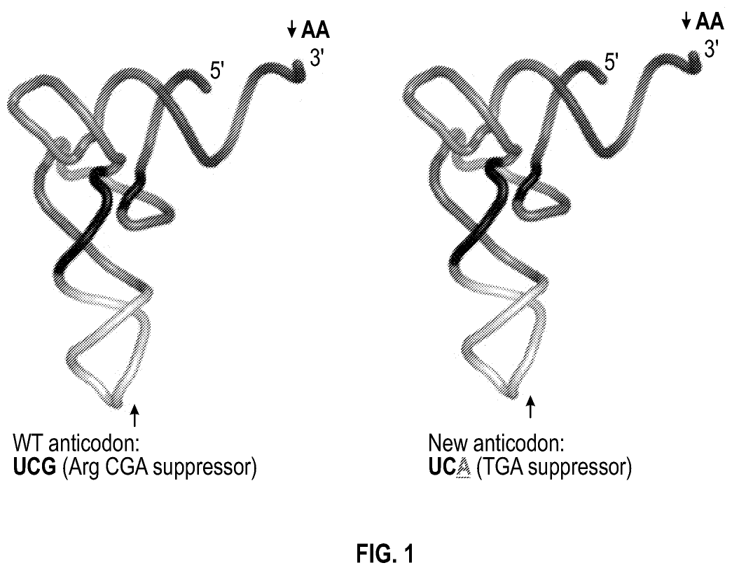

There are multiple tRNA genes for each amino acid type, each with unique sequences (isodecoders) for a given cognate amino acid (isoacceptors), leading to >400 tRNAs annotated in the human genome (56, 57). Screening for individual (human) anticodon-mutated tRNAs that retain suppression efficacy of PTCs in mammalian cells has been done (16). This approach has identified numerous suppressor tRNAs. FIG. 1, Left, shows the folded tRNA structure with the anticodon region shown as a loop at the bottom of the structure. In this example, UCG is the wildtype tRNA anticodon for decoding the CGA Arginine codon. Right, mutation of UCG-to-UCA within the anticodon (underline) converts this tRNA to a UGA (RNA) stop codon suppressor.

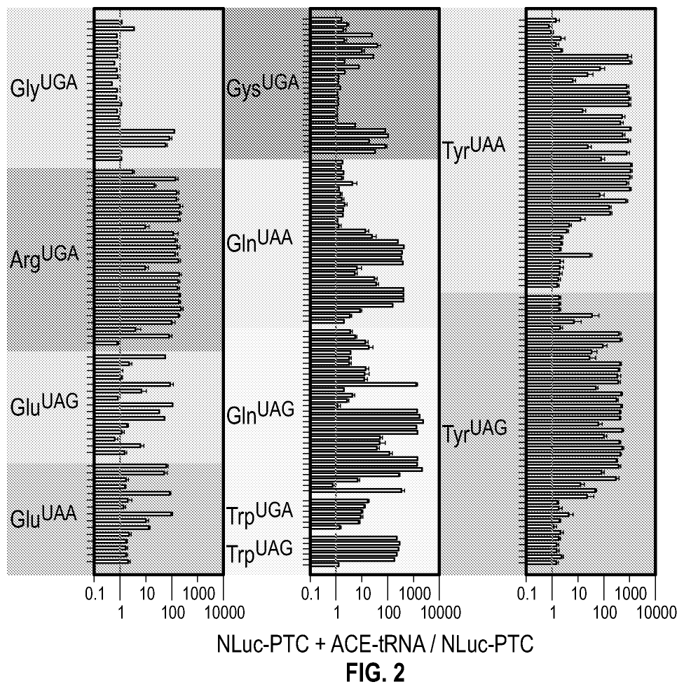

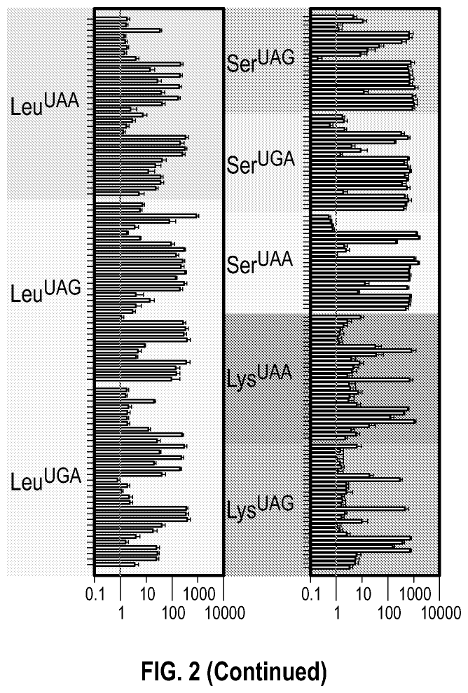

tRNA suppressors for every known human disease-causing PTC have been identified (FIG. 2). Data are shown in Log 10 scale (below) in terms of normalized fold-over background NLuc luminescence with each bar an individual engineered tRNA sequence. There are multiple unique tRNA sequences (isodecoders) within each tRNA family, where in each case the anticodon editing type is superscripted. Dashed line indicates baseline tRNA suppression activity. Each tRNA dataset were obtained in triplicates and are displayed at average±SEM.

Nucleotide sequences encoding several hundred human tRNAs are known and generally available to those of skill in the art through sources such as GenBank. The structure of tRNAs is highly conserved, and tRNAs can be functional across species. Thus, bacterial or other eukaryotic tRNA sequences are also potential sources for the tRNAs of the present disclosure. The determination of whether a particular tRNA is functional as desired, such as in a desired mammalian cell, can be ascertained as described herein or through other experimentation that will be apparent to one of ordinary skill in the art with the benefit of the teachings provided herein. In some embodiments, the tRNA sequences may be any of the sequences provided in PCT/US2018/059065, WO2019/090154, WO2019/090169, and Lueck et al., Nature Communications 10, 822, 2019, the disclosure of the tRNAs and sequences of which are incorporated herein by reference. Thus, in one embodiment of any one of the compositions or methods provided herein the tRNA(s) are selected from any one of the foregoing.

The tRNAs and nucleotide sequences encoding the tRNAs can be generated synthetically. The tRNAs can be delivered as DNA or RNA (e.g., viral vector (e.g., AAV)/minicircle/nanoparticles). Data of viral and non-viral delivery methods of cDNA and RNA to deliver nucleic acids into retinal cells are provided, providing proof of principle. The tRNAs generally are small (˜100 bp). The following are noted:
1. The rescued protein levels of the PTC-containing genes are based on the activity of the endogenous gene promoter, and mRNA present within the targeted cell thus will be subject to normal cellular regulatory events. This is important for ion channel genes that require tightly regulated expression paradigms.
2. The tRNA suppression approach is “positionally agnostic,” that is, an in-frame termination codon can be repaired regardless of its location or context within a gene of interest or any gene. Further, preliminary data suggest that the size of the PTC-containing gene does not impact rescue efficacy, thus allowing for the targeting of large genes that exceed viral payloads, such as cytoskeletal proteins, pumps, transporters, and ion channels.

The tRNA therapeutics may delivered as DNA, such as cDNA, or RNA, in some embodiments of any one of the compositions or methods provided herein. The tRNA(s) may be in the form of any suitable recombinant plasmid that comprises a heterologous nucleic acid sequence to be delivered to a target cell or subject, either in vitro or in vivo. The heterologous nucleic acid sequence encodes a gene product (e.g., a tRNA) of interest for the purposes of, for example, any one of the uses or methods provided herein including disease treatment, and may, optionally, be in the form of an expression cassette. The term “recombinant” refers to a polynucleotide which either does not occur in nature or is linked to another polynucleotide in an arrangement not found in nature. The term “heterologous,” as used herein refers to a nucleic acid sequence obtained or derived from a genetically distinct entity from the rest of the entity to which it is being compared.

In certain embodiments of any one of the compositions or methods provided, the tRNA is encoded in an expression cassette. Thus, the present invention also provides an expression cassette comprising a sequence encoding a tRNA as provided herein. In certain embodiments, the expression cassette further contains a promoter. In certain embodiments, the promoter is a regulatable promoter. In certain embodiments, the promoter is a constitutive promoter. The promoter to drive expression of the sequence encoding the tRNA to be delivered can be any desired promoter, selected by known considerations, such as the level of expression of a nucleic acid functionally linked to the promoter and the cell type in which the vector is to be used. Promoters can be an exogenous or an endogenous promoter.

“Expression cassette” as used herein means a nucleic acid sequence capable of directing expression of a particular nucleotide sequence in an appropriate cell, which may include a promoter operably linked to the nucleotide sequence of interest that may be operably linked to termination signals. The expression cassette including the nucleotide sequence of interest may be chimeric. The expression cassette may also be one that is naturally occurring but has been obtained in a recombinant form useful for heterologous expression. The expression of the nucleotide sequence in the expression cassette may be under the control of a constitutive promoter or of a regulatable promoter. The expression cassette may be or contained in a vector.

Typically, the expression cassette includes the heterologous gene together with a promoter to control transcription of the new gene. The promoter characteristically has a specific nucleotide sequence necessary to initiate transcription. Optionally, the expression cassette further includes additional sequences (i.e., enhancers) required to obtain the desired gene transcription activity. The heterologous gene may be introduced into a nucleic acid immediately downstream from the promoter so that the promoter and coding sequence are operatively linked so as to permit transcription of the coding sequence.

“Operably linked” refers to the association of nucleic acid sequences on single nucleic acid fragment so that the function of one of the sequences is affected by another. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence.

In addition to at least one promoter and at least one heterologous nucleic acid, the expression cassette may include a selection gene, for example, green fluorescent protein (GFP), for facilitating selection of cells that have been transfected with the expression cassette. Alternatively, the cells are transfected with two or more expression cassettes, at least one containing the gene(s) encoding the tRNA, the other containing a selection gene. The selection of a suitable promoter, enhancer, selection gene and/or signal sequence is deemed to be within the scope of one of ordinary skill in the art without undue experimentation.

The tRNA therapeutic can be delivered in the form of a vector. Vectors include but are not limited to eukaryotic vectors, prokaryotic vectors (such as for example bacterial vectors) and viral vectors including, but not limited to, retroviral vectors, adenoviral vectors, adeno-associated viral vectors, lentivirus vectors (human and other including porcine), Herpes virus vectors, Epstein-Barr viral vectors, SV40 virus vectors, pox virus vectors, and pseudotyped viral vectors.

Successful delivery and function of tRNAs as provided herein can occur when in the form of minicircles. The minicircle technology uses a small excised, bacterial DNA-free circular DNA fragment that results in the efficient delivery of any insert size for both in vitro and in vivo applications. The term “minicircle”, as used herein, refers to small circular DNA fragments that are largely or completely free of non-essential prokaryotic elements. Minicircles include circular forms of DNA without prokaryotic elements and/or in which prokaryotic elements have been removed. Minicircles can be from a parental plasmid where bacterial DNA sequences have been excised.

The use of minicircles has a strong safety profile, and the preparation of minicircles has been described in the art (e.g., in Nehlsen et al., Gene Ther. Mol. Biol. 10:233-244, 2006; and Kay et al., Nature Biotechnology. 28:1287-1289, 2010). The preparation can, for example, follow a two-step procedure: (i) production of a ‘parental plasmid’ (bacterial plasmid with eukaryotic inserts); and (ii) induction of a site-specific recombinase at the end of this process. These steps can be followed by the excision of prokaryotic vector parts via recombinase-target sequences and recovery by capillary gel electrophoresis.

As a nonlimiting example, a minicircle may be produced as follows. An expression cassette, which comprises the polynucleotide coding sequence along with regulatory elements for its expression, is flanked by attachment sites for a recombinase. A sequence encoding the recombinase is located outside of the expression cassette and includes elements for inducible expression (such as, for example, an inducible promoter). Upon induction of recombinase expression, the vector DNA is recombined, resulting in two distinct circular DNA molecules. One of the circular DNA molecules is relatively small, forming a minicircle that comprises the expression cassette for the polynucleotide; this minicircle DNA vector is devoid of any bacterial DNA sequences. The second circular DNA sequence contains the remaining vector sequence, including the bacterial sequences and the sequence encoding the recombinase. The minicircle DNA containing the polynucleotide sequence can then be separately isolated and purified. In some embodiments, a minicircle DNA vector may be produced using plasmids similar to pBAD.ϕ.C31.hFIX and pBAD.ϕ.C31.RHB. See, e.g., Chen et al. (2003) Mol. Ther. 8:495-500, or as otherwise provided herein.

Examples of recombinases that may be used for creating a minicircle include, but are not limited to, Streptomyces bacteriophage ϕ31 integrase, Cre recombinase, and the 2 integrase/DNA topoisomerase IV complex. Each of these recombinases catalyzes recombination between distinct sites. For example, ϕ31 integrase catalyzes recombination between corresponding attP and attB sites, Cre recombinase catalyzes recombination between loxP sites, and the 2 integrase/DNA topoisomerase IV complex catalyzes recombination between bacteriophage 2 attP and attB sites.

Published U.S. application No. 20170342424 also describes a system making use of a parent plasmid which is exposed to an enzyme which causes recombination at recombination sites, thereby forming a (i) minicircle including the polynucleotide sequence and (ii) miniplasmid comprising the remainder of the parent plasmid. One recombination site is modified at the 5′ end such that its reaction with the enzyme is less efficient than the wild type site, and the other recombination site is modified at the 3′ end such that its reaction with the enzyme is less efficient than the wild type site, and the other recombination site is modified at the 3′ end such that its reaction with the enzyme is less efficient than the wild type site, both modified sites being located in the minicircle after recombination.

Removal of prokaryotic sequences ideally should be efficient, using the smallest possible excision site, while creating supercoiled DNA minicircles which consist solely of gene expression elements under appropriate—preferably mammalian—control regions. Some techniques for minicircle production use bacterial phage lambda (2) integrase mediated recombination to produce minicircle DNA. See, for example, Darquet, et al. 1997 Gene Ther 4(12): 1341-9; Darquet et al. 1999 Gene Ther 6(2): 209-18; and Kreiss, et al. 1998 Appl Micbiol Biotechnol 49(5): 560-7).

Kits for producing minicircle DNA are known in the art and are commercially available (System Biosciences, Inc., Palo Alto, Calif.). For example, a MC-Easy™ (Cat #MN920A-1, SBI System Biosciences) Minicircle DNA production kit can be used to obtain high-quality minicircle DNA. Information on minicircle DNA is provided in Dietz et al., Vector Engineering and Delivery Molecular Therapy (2013); 21 8, 1526-1535 and Hou et al., Molecular Therapy—Methods & Clinical Development, Article number: 14062 (2015) doi: 10.1038/mtm.2014.62. More information on Minicircles is provided in Chen Z Y, He C Y, Ehrhardt A, Kay M A. Mol Ther. 2003 September; 8(3): 495-500 and Minicircle DNA vectors achieve sustained expression reflected by active chromatin and transcriptional level. Gracey Maniar L E, Maniar J M, Chen Z Y, Lu J, Fire A Z, Kay M A. Mol Ther. 2013 January; 21(1): 131.

Any of the compositions provided herein can be placed in contact with, administered to or introduced into a cell with genetic transfer methods, such as transfection. Thus, any of the compositions provided herein can be included with or in a delivery vehicle. The compositions provided herein can be contacted with cells or delivered or administered to a subject within a particle, such as a nanoparticle. A particle, such as a nanoparticle (NP), can be, but is not limited to, lipid-based nanoparticles (also referred to herein as lipid nanoparticles, i.e., nanoparticles where the majority of the material that makes up their structure are lipids). A particle can also be polymer-based (also referred to herein as polymer or polymeric nanoparticles, i.e., nanoparticles where the majority of the material that makes up their structure are polymers) (e.g., polyplexes). The lipid or polymer-based NPs can be formed through electrostatic interactions between the negatively charged payloads and the positively charged carrier materials such as cationic lipids and cationic polymers.

In one embodiment of any one of the compositions or methods provided herein, the nanoparticles are silica nanoparticles (SNPs), such as glutathione (GSH)-responsive silica nanoparticles. The SNPs can have (1) high payload loading content and loading efficiency, (2) versatile surface chemistry to allow physical coating or chemical conjugation of various moieties to facilitate transport to the outer retina after intravitreal injections and to enable targeting, (3) small SNP sizes (e.g., dH<50 nm) which are desirable for tissue penetration and diffusion, (4) excellent in vitro and in vivo stability, (5) efficient endo/lysosomal escape, (6) rapid stimulus-responsive payload release once inside the cytosol, (7) excellent biocompatibility and negligible immunogenicity, (8) ability to scale up, (9) ease of handling, storage, and transport or a combination thereof. In one embodiment of any one of the compositions or methods provided herein, the SNPs are negatively charged, hyaluronic acid (HA)-coated SNPs that can exhibit enhanced penetration to the outer retina after intravitreal injections. In another embodiment of any one of the compositions or methods provided herein, to enhance the cellular uptake of SNPs by target cells, molecules, such as 11cRal or ATRA, can be conjugated on the surface of the SNPs for delivery.

The present disclosure provides methods for delivering one or more tRNA(s) or compositions provided herein to a subject or to cells of a subject. The tRNA(s) or composition as described herein can be delivered to a cell in vivo or in vitro. Administration to the cell can be accomplished by any means, including simply contacting the cell. The contact with the cells can be for any desired length of time. The cells can include any desired cell in humans as well as other large (non-rodent) mammals, such as primates, horse, sheep, goat, pig, and dog. Any one of the subjects provided herein can be a human or other mammal. The term “mammal” includes, but is not limited to, humans, mice, rats, guinea pigs, monkeys, dogs, cats, horses, cows, pigs, and sheep. Any one of the subjects provided herein can have any one of the diseases or conditions provided herein and/or can benefit from treatment with any one of the tRNAs provided herein.

Suitable methods for delivery and introduction into a subject are also provided or otherwise understood in the art. In one embodiment of any one of the compositions or methods provided herein, pharmaceutical compositions comprise sufficient genetic material to produce a therapeutically effective amount of the tRNA(s) of interest. The tRNAs or genetic material that encodes the tRNA(s) can be delivered in an effective amount, and into a cell, such as one with endogenous tRNA synthetase. A tRNA synthetase is considered to be “endogenous” to a cell if it is present in the cell into which a tRNA is introduced according to the present invention. As will be the apparent to those of ordinary skill in the art, a tRNA synthetase may be considered to be endogenous for these purposes whether it is naturally found in cells of the relevant type, or whether the particular cell at issue has been engineered or otherwise manipulated by the hand of man to contain or express it.

In some embodiments, the tRNA or composition as provided herein can be formulated in a pharmaceutical composition. The pharmaceutical compositions may also contain a pharmaceutically acceptable excipient. Such excipients include any pharmaceutical agent that may be administered without undue toxicity. Pharmaceutically acceptable excipients include, but are not limited to, sorbitol, Tween80, and liquids such as water, saline, glycerol and ethanol. Pharmaceutically acceptable salts can be included therein, for example, mineral acid salts such as hydrochlorides, hydrobromides, phosphates, sulfates, and the like; and the salts of organic acids such as acetates, propionates, malonates, benzoates, and the like. Additionally, auxiliary substances, such as wetting or emulsifying agents, pH buffering substances, and the like, may be present in such vehicles. A thorough discussion of pharmaceutically acceptable excipients is available in Remington's Pharmaceutical Sciences (Mack Pub. Co., N.J. 1991).

As is apparent to those skilled in the art in view of the teachings of this specification, an effective amount of the tRNAs provided may be empirically determined. Administration can be effected in one dose, continuously or intermittently, throughout the course of treatment. The administration may be essentially continuous over a preselected period of time or may be in a series of spaced doses. Methods of determining the most effective means and dosages of administration may vary with the composition of the therapy, target cells, and the subject being treated, etc. Single and multiple administrations can be carried out with the dose level and pattern being selected by the treating physician.

Vehicles including water, aqueous saline, artificial CSF, or other known substances can be employed with the subject invention. To prepare a formulation, the purified composition can be isolated. The composition may then be adjusted to an appropriate concentration and packaged for use.

As used herein, the terms “treat” and “treatment” refer to both therapeutic treatment and measures that can alleviate symptoms or provide some benefit to a subject. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. Those in need of treatment include those already with the condition, disease or disorder.

The phrase “therapeutically effective amount” means an amount of one or more compounds or a composition of the present invention that (i) treats the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein. The tRNA(s) or composition described herein may be administered so as to result in a reduction in at least one symptom associated with a disease or disorder. The amount administered will vary depending on various factors including, but not limited to, the composition chosen, the particular disease, the weight, the physical condition, and the age of the mammal. Such factors can be readily determined by the clinician employing animal models or other test systems that are known to the art.

One or more suitable unit dosage forms having one or more tRNA(s) or composition of the invention may be formulated and can be administered by a variety of routes. When the agents of the invention are prepared for administration, they may be combined with a pharmaceutically acceptable carrier, diluent or excipient to form a pharmaceutical formulation, or unit dosage form. The total active ingredients in such formulations include from 0.1 to 99.9% by weight of the formulation. A “pharmaceutically acceptable” is a carrier, diluent, excipient, and/or salt that is compatible with the other ingredients of the formulation, and not deleterious to the recipient thereof. Pharmaceutical formulations can be prepared by procedures known in the art using well-known and readily available ingredients. The agents of the invention can also be formulated as solutions appropriate for administration. The pharmaceutical formulations of the agents of the invention can also take the form of an aqueous or anhydrous solution or dispersion, or alternatively the form of an emulsion or suspension.

Thus, the agent(s) may be formulated for administration and may be presented in unit dose form in ampules, pre-filled syringes, small volume infusion containers or in multi-dose containers with an added preservative. The active ingredients may take such forms as suspensions, solutions, or emulsions in oily or aqueous vehicles, and may contain formulatory agents such as suspending, stabilizing and/or dispersing agents. Alternatively, the active ingredients may be in a suitable vehicle, e.g., sterile, pyrogen-free water, before use. It will be appreciated that the unit content of active ingredient or ingredients contained in an individual dose of each dosage form need not in itself constitute an effective amount for treating the particular indication or disease since the necessary effective amount can be reached by administration of a plurality of dosage units. Moreover, the effective amount may be achieved using less than the dose in the dosage form, either individually, or in a series of administrations.

The pharmaceutical formulations of the present invention may include, as optional ingredients, pharmaceutically acceptable carriers, diluents, solubilizing or emulsifying agents, and salts of the type that are well-known in the art. Specific non-limiting examples of the carriers and/or diluents that are useful in the pharmaceutical formulations of the present invention include water and physiologically acceptable buffered saline solutions such as phosphate buffered saline solutions pH 7.0-8.0 and water.

Although the foregoing specification and examples fully disclose and enable the present invention, they are not intended to limit the scope of the invention, which is defined by the claims appended hereto.

All publications, patents and patent applications are incorporated herein by reference. While in the foregoing specification this invention has been described in relation to certain embodiments thereof, and many details have been set forth for purposes of illustration, it will be apparent to those skilled in the art that the invention is susceptible to additional embodiments and that certain of the details described herein may be varied considerably without departing from the basic principles of the invention.

The use of the terms “a” and “an” and “the” and similar referents in the context of describing the invention are to be construed to cover both the singular and the plural, unless otherwise indicated herein or clearly contradicted by context. The terms “comprising,” “having,” “including,” and “containing” are to be construed as open-ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Embodiments of this invention are described herein, including the best mode known to the inventors for carrying out the invention. Variations of those embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### EXAMPLES

### Example 1: Restoring Vision with High-Fidelity Nonsense Codon Correction

Genetic blindness is a significant health, societal, and economic burden that afflicts millions of patients globally. While there are available adeno-associated viral (AAV) gene therapy approaches, such as Luxturna®, these address rare forms of blindness with very limited indications. Unfortunately, many proteins that are essential to visual processing, e.g., ion channels, exceed the payload size of currently available AAV delivery systems. Further, mutations which produce premature termination codons (PTC), that is, mutations which convert ‘sense’ codons to one of the three nonsense codons (TGA, TAA, TAG), represent a unique challenge given that they often produce more severe phenotypes, are found throughout essential proteins, and are more numerous in large genes. Thus, the nonsense codon PTC itself is a common molecular basis for inherited blindness.

Herein, provided is a therapy approach that utilizes transfer RNA (tRNA) molecules for the high-fidelity in vivo repair of PTC(s). The tRNA may be an “ace-tRNA”, in some embodiments. Data indicate that such tRNAs can be expressed as DNA or through viral vectors, such as AAV, in retinal cells where they display potent biological PTC repair activity with undetectable toxicology profiles. PTC-containing genes cause a myriad of blindness conditions afflicting—in total—thousands of patients. Both viral and non-viral delivery of tRNAs and the in vivo repair activities in patient-derived iPSC-retinal pigment epithelium (RPE) cells, photoreceptor (PR) organoids, intravitreal injections in mouse models of genetic blindness, and biosafety and bioavailability in non-human primates are or have been assessed.

### Importance of Ion Channels in Inherited Retinal Disease

Ion channels are expressed in retinal neurons, glia, and epithelial cells to maintain membrane potential, signal transduction, ion transport, and volume regulation. Interactions between PR and RPE is responsible for collecting and processing visual stimuli, through the synchronized function of multiple ion channels. Ion channel-based disorders-collectively termed “ion channelopathies”—are central to the development of therapeutics for blindness because they 1) affect both RPE and PRs, 2) benefit from established experimental endpoints in vitro and in vivo readouts (i.e., one can directly measure channel function with electrophysiological approached—not trivial with in vitro systems), 3) they are particularly sensitive to missense mutations, so they generally need not just read-through, but wildtype AA insertion at the PTC site and 4) overall represent an excellent “category” of diseases to test the therapeutic potential for tRNAs.

Provided herein is a therapy approach for the repair of premature termination codons in any one of the genes described herein. This method relies on transfer RNA (tRNA) to suppress premature termination codons. Data show that the approach displays high-efficiency and high-fidelity PTC repair regardless of the cell, gene, or site of repair. High-fidelity repair: the correct amino acid is repositioned at the PTC (as determined by mass spectrometry) in combination with minimal interactions at 3′ protein termination stop codons, as shown by whole-exome ribosomal profiling. Each ace-tRNA for a given amino acid was synthesized as an oligo containing nucleotide changes to the anticodon region and cloned into a single expression vector, which included a nanoluciferase-STOP reporter gene, with the STOP (TGA, TAA, TAG) corresponding to the inserted tRNA. Each construct was transfected in a 96-well format and measured for luciferase activity on a SpectraMax i3 plate reader after 24 hours, see (16) for details. Each bar represents a unique tRNA sequence. This screen identified multiple anticodon-edited ace-tRNAs for each of the ten amino acids vulnerable to stop codon conversion and by stop codon type, with suppression coverage for all three stop codons, thus providing therapeutic coverage of every human disease-causing PTC caused by single nucleotide changes.

### Very Low Suppression Activity at Protein Termination Codons

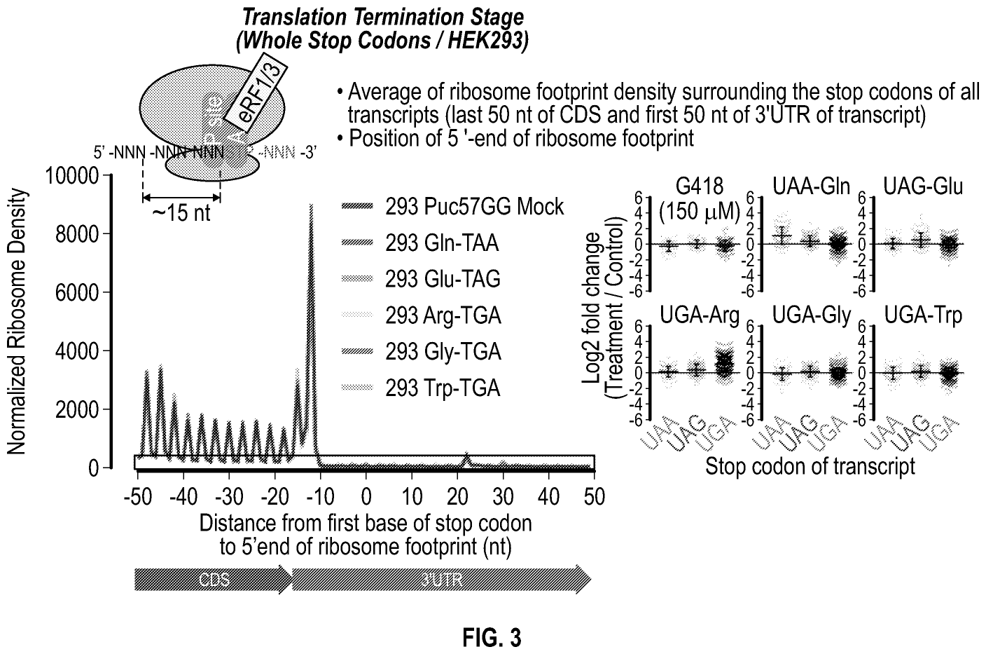

Riboprofiling was used to address the possibility that suppressor tRNAs that show effective repair activity of in-frame PTCs may also induce a global read-through of native stop codons, which would be undesirable for a therapy. Riboprofiling, aka ribosomal profile assay, allows for the simultaneous detection of the location of actively transcribing ribosomes within a cell. If the suppressor tRNAs are promoting read-through at protein termination stop codon, one would be able to measure this as ribosomes being present on the 3′ UTR region of the RNA after the stop codon. To assess such “off-target” suppression of 3′ stop codons a transcriptome-wide quantitative profile was performed of actively engaged ribosomes on all cellular transcripts by generating libraries of ribosome footprints from HEK293 cells expressing exogenous tRNAs or a control mock plasmid (puc57GG). FIG. 3 shows ribosome footprint densities for RNA purified from HEK cells exposed to G418 or expressing each of the five indicated tRNA types. Only transcripts with a minimum threshold of 5 RPKM in the coding sequence and 0.5 RPKM in the 3′UTR in two replicate libraries were included for the quantitation comparison (254 transcripts in G418 and 495-748 transcriptsin tRNAs). The ‘saw-tooth’ plot represents data from all transcripts. It has been averaged for ribosomal positioning, making apparent the reading frame as the ribosome translates the mRNA, pausing at each codon creating a density at the tip of the spike and then ribosomal stalling at the end of the reading frame (seen as the significant spike). Note very low/no ribosomal activity seen after the “−10” coordinate, which demarks the stop codon region and the beginning of the 3′UTR (untranslated region), are shown by the arrows below, respectively. The inset on the right shows these data for each stop codon and ace-tRNA suppressor type. These were compared with controls (log 2-fold change) on 3′UTR regions, i.e., evidence of ribosomes appearing on 3′UTR regions would suggest read-through of the protein termination codon. The ribosome profiling data argue that efficiency of native stop codon suppression by these engineered tRNAs is low or undetectable, and is markedly less than the level of in-frame PTC suppression. One possibility for this observation is that the Eukaryotic translation termination factor 1 (eRF1) outcompetes the suppressor ace-tRNA.

### High-Fidelity Encoding and PTC Suppression

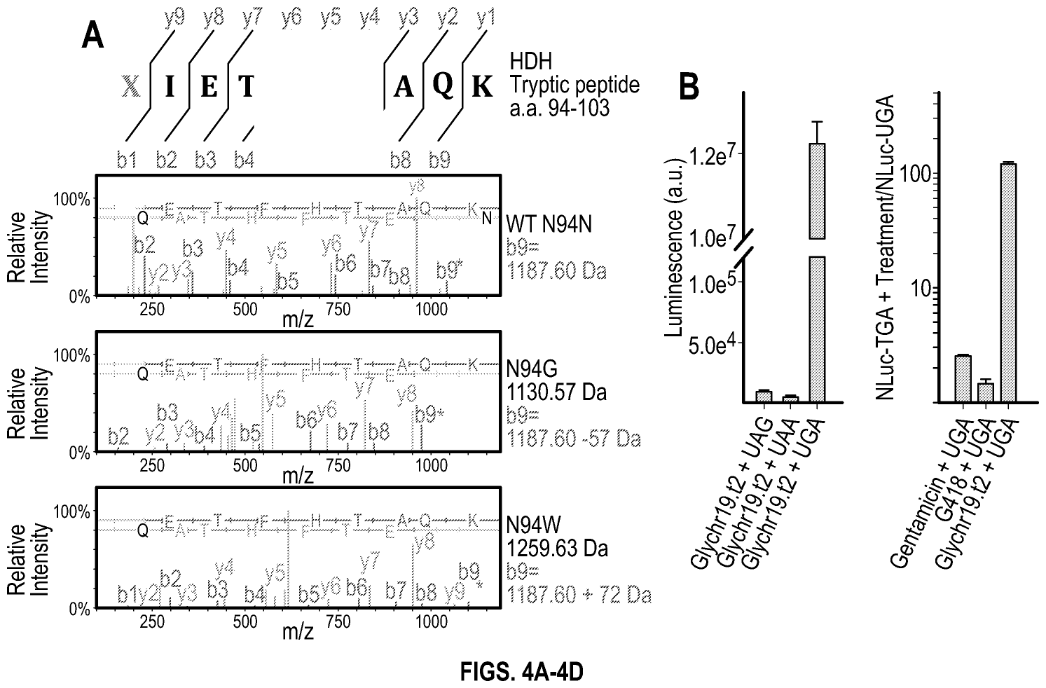

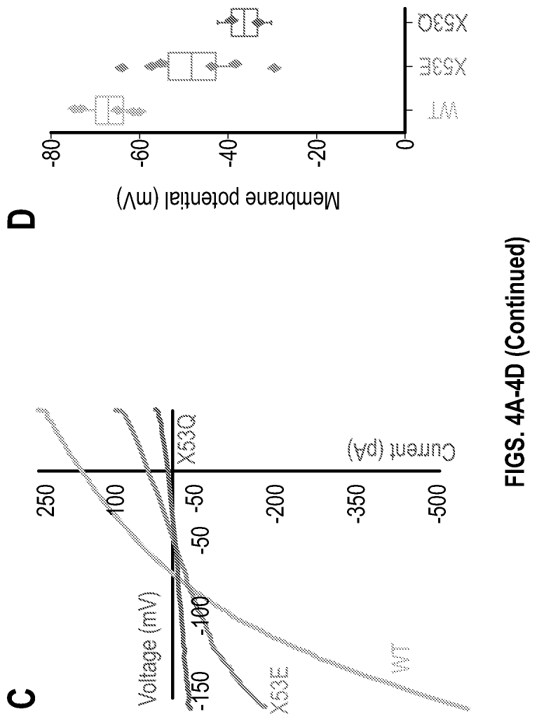

Whether the ace-tRNAs identified in the screen were functionalized to suppress PTC at the expense of aminoacylation stringency by the cognate aminoacyl-tRNA synthetase was considered. To this end, mass spectrometry was used to examine PTC suppression in a model soluble protein, histidinol dehydrogenase (HDH), FIG. 4A. Here, an in-frame TGA codon was introduced at asparagine 94 (N94) co-expressed in HEK293 cells in tandem with plasmids encoding Glychr19.trna2 or Trpchr17.trna39 ace-tRNAs, two representative tRNA from the screen. Searches of the MS data identified the modification of Asn to Trp (+72 Da) for Trp chr17.trna39 and (−57 Da) for Glychr19.trna2, thus confirming the accurate encoding of the cognate amino acid for each ace-tRNA type in >98% of the peptides identified. Codon edited tRNAs displayed selectivity for the UGA stop codon, over UAA and UAG, FIG. 4B, left panel. The suppressor tRNA outperform the read-through compounds gentamicin and G418, FIG. 4B, right panel. It has also been established that missense mutations in ion channels produce altered gating and expression profiles, and are often a cause of disease phenotypes. An example of this is shown in FIGS. 4C and 4D where the Kir7.1 potassium channel (isoform) was tested for tolerance of the W53E and W53Q mutations, with W53X being a known LCA PTC mutation and Gln and Glu being the ‘near-cognate’ amino acids that would be encoded if using read-through drugs, such as Ataluren. Here, the missense containing channels have reduced current density and perturbed voltage-dependent gating.

### Potent Amino Acid Encoding and Protein Rescue Efficiency Regardless of PTC or PTC Position

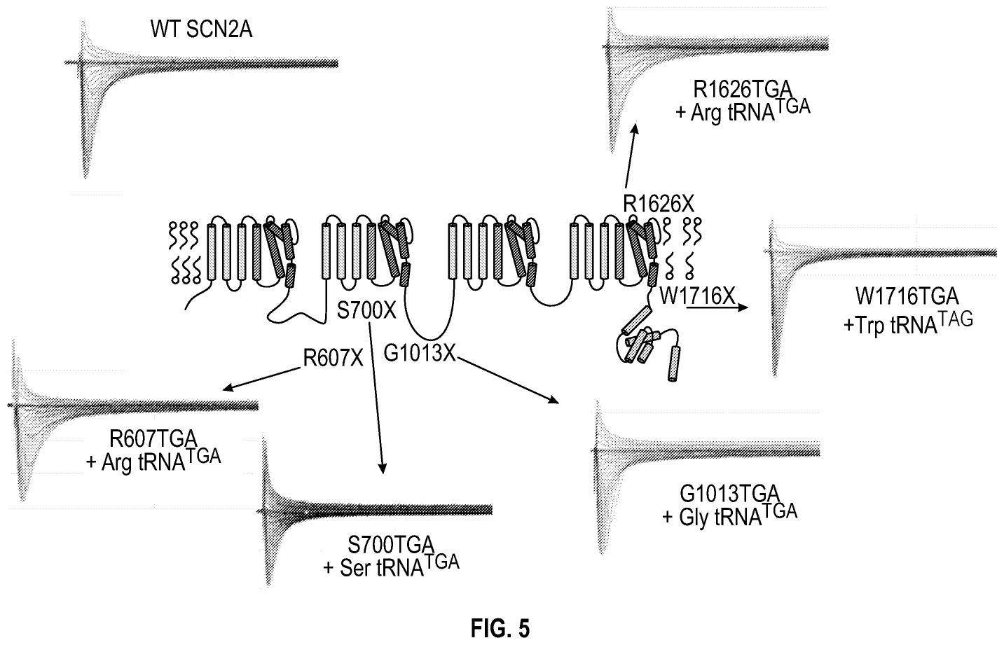

In order to experimentally assess the ability of tRNA identified in the screen for suppression of bona fide human disease-causing PTC, the SCN2A sodium channel transiently expressed in HEK cells was used. Five human different kinds of PTC mutations that are found throughout the SCN2A gene that are known to cause autism and developmental delay were chosen. For each, a tRNA from the expression library was matched with the activity for the desired PTC stop codon and amino acid type. SCN2A PTC channel cDNA was then expressed alone or with a construct expressing the indicated tRNA type and GFP-X reporter to mark expressing cells for patch-clamp analysis. SCN2A PTC cDNA expressed with a WT GFP did not display sodium channel activity consistent with a truncated, non-functional Nav1.2 protein. However, co-expression with the indicated ace-tRNA construct for each PTC produced robust sodium channel activity with a current density (metric of the number of channels expressed) and gating characteristics that were indistinguishable from WT SCN2A channels (FIG. 5, upper left). In each example the rescued currents were similar to WT expression regardless of PTC context (beginning, middle, or end of gene), PTC kind (TGA or TAG), or the encoded amino acid.

### Preparation and Characterization of the SNPs

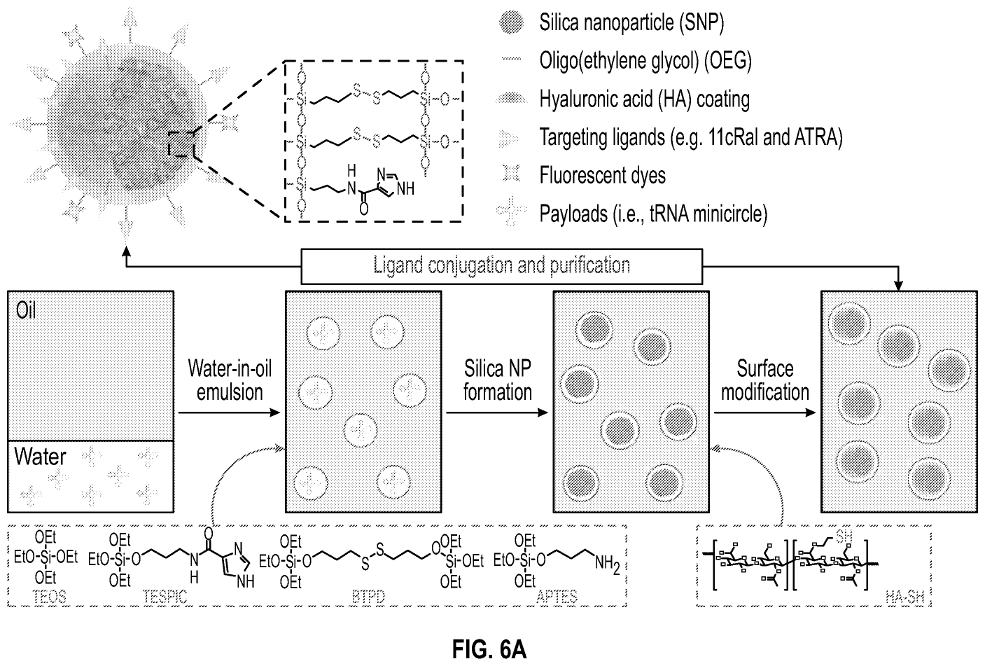

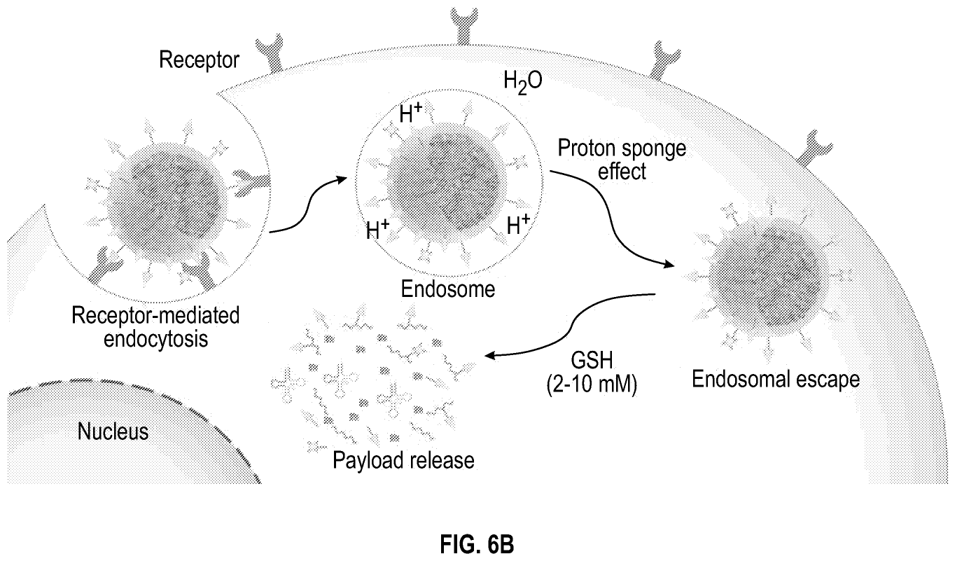

SNPs were synthesized using a water-in-oil microemulsion method. The aqueous solution containing the payload (e.g., pDNA, ace-tRNA encoding-cDNA, RNA or Cas9 RNP) was mixed with N-(3-(triethoxysilyl) propyl)-1H-imidazole-4-carboxamide (TESPIC), tetraethyl orthosilicate (TEOS) and bis[3-(triethoxysilyl)propyl]disulfide (BTPD), and then added to the organic solution composed of Triton X-100, n-hexanol and cyclohexane. Thereafter, an ammonia aqueous solution was added into the mixture to initiate the reaction under stirring overnight. The SNPs were precipitated by acetone and recovered by centrifugation and purified by washing with ethanol and water. The poly(ethylene glycol) (PEG)-conjugated SNPs were prepared by reacting SNPs with PEG-silane and purified by washing with water and centrifugation. The resulting PEGylated SNPs were then modified with a desirable amount of targeting ligands, such as ATRA or CPP (FIGS. 6A-6C).

The morphology (spherical NPs with a size around 40-50 nm) and zeta-potential (i.e., surface charges) of the SNPs were assessed by transmission electron microscopy (TEM) and dynamic light scattering (DLS). The loading efficiency (˜95%) and loading content (˜10 wt %) of the nucleic acids were determined by Nanodrop and/or the bicinchoninic acid assay (BCA assay). The SNPs are prepared using a water-in-oil microemulsion process, thereby leading to high encapsulation efficiencies (˜95%) and high loading contents (˜10 wt %) for nucleic acids. The size of the SNPs can also be conveniently controlled during the microemulsion process to yield SNPs with different sizes, important in determining optimal dimension for retina penetration through intravitreal delivery. The hydrodynamic diameter of the SNPs ranges from 40 to 50 nm. In general, smaller NPs have higher diffusivity and can be more readily taken up by target cells. Due to their covalent nature, the SNPs also have excellent stability in the extracellular space. Imidazole functional groups can be integrated into the SNP, which can facilitate the SNP escape from endo/lysosomes via the proton sponge effect. Disulfide crosslinkers are incorporated into the silica network, which can break down in cytosol due to the high GSH concentration (2-10 mM), thus triggering a fast release of the payloads.

The SNPs demonstrated high in vitro transfection efficiencies in HEK 293T cells for pDNA and mRNA delivery as well as high editing efficiency for CRISPR RNP delivery. The SNP also exhibited significant cellular uptake in iPSC-RPE cells). Moreover, subretinally injected Cre mRNA or Cas9 RNP-loaded SNPs conjugated with all-trans-retinoic acid (ATRA, targeting RPE cells) or 11-cis-retinal (11cRal, targeting PRs) induced significant gene editing in RPE cells or PRs in vivo. The inter-photoreceptor retinoid-binding protein, a major protein in the inter-photoreceptor matrix, selectively transports 11cRal to PR and ATRA to the RPE, respectively (58, 59). The SNP-ATRA can also deliver tRNA-encoding cDNA in vivo, leading to robust ace-tRNA transfection in RPE cells in mice. In addition, no apparent cytotoxicity or tissue damage/inflammation were induced by SNP treatments in cells or animals, suggesting that SNPs have good biocompatibility.

The versatility of the SNP in terms of (1) the types of payload it can deliver (e.g., tRNA minicircle, plasmid DNA, and proteins), (2) the types of surface functionalization it enables, (3) its basic characteristics (e.g., high encapsulation efficiency and high payload loading content, small and controllable sizes, excellent biocompatibility, high stability in the extracellular space, and GSH-triggered payload release inside cytosol), (4) its proven capability to induce high transfection/editing efficiencies for multiple types of payload to various kinds of cells both in vitro and in vivo, or a combination thereof, makes it a desirable nanoplatform to be further engineered for intravitreal delivery of tRNA, such as in the form of minicircles, to target PR or RPE cells.

### Delivery Efficiency of Nucleic Acids and CRISPR RNP by SNPs In Vitro

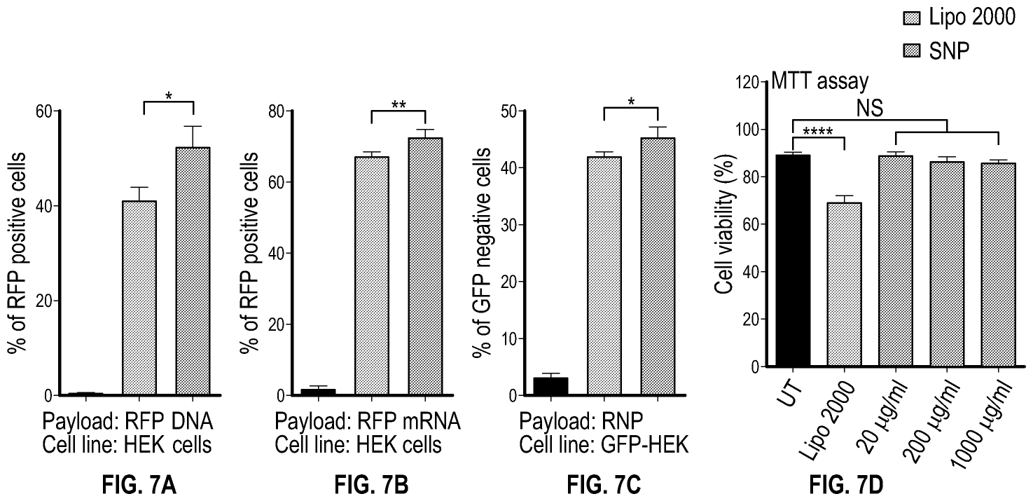

The SNP formulation was optimized in vitro using red fluorescence protein (RFP)-encoding plasmid DNA and mRNA as the payload, respectively. The incorporation ratio of imidazole-containing TESPIC in the formulation and the surface charge of the SNPs were both optimized in HEK 293T cells, with significantly higher transfection efficiency than the commercially available transfection agent Lipofectamine 2000 (Lipo 2000) (FIGS. 7A and B), indicating the efficient delivery of nucleic acids by SNPs. To investigate the genome-editing efficiency of Cas9 RNP-loaded SNPs, a sgRNA targeting the GFP gene in a transgenic GFP-expressing HEK 293T cell line was used. As shown in FIG. 7C, SNPs exhibited a significantly higher GFP-knockout efficiency than Lipo 2000. For evaluation of the biocompatibility of SNPs, the HEK 293T cells were treated with SNPs, and the cell viability was investigated by an MTT assay (FIG. 7D). These results indicate that the SNPs can efficiently deliver a diverse range of hydrophilic payloads in vitro, including nucleic acids, with minimal cytotoxicity.

### Delivery Efficiency of Nucleic Acids and CRISPR RNP by SNPs In Vivo

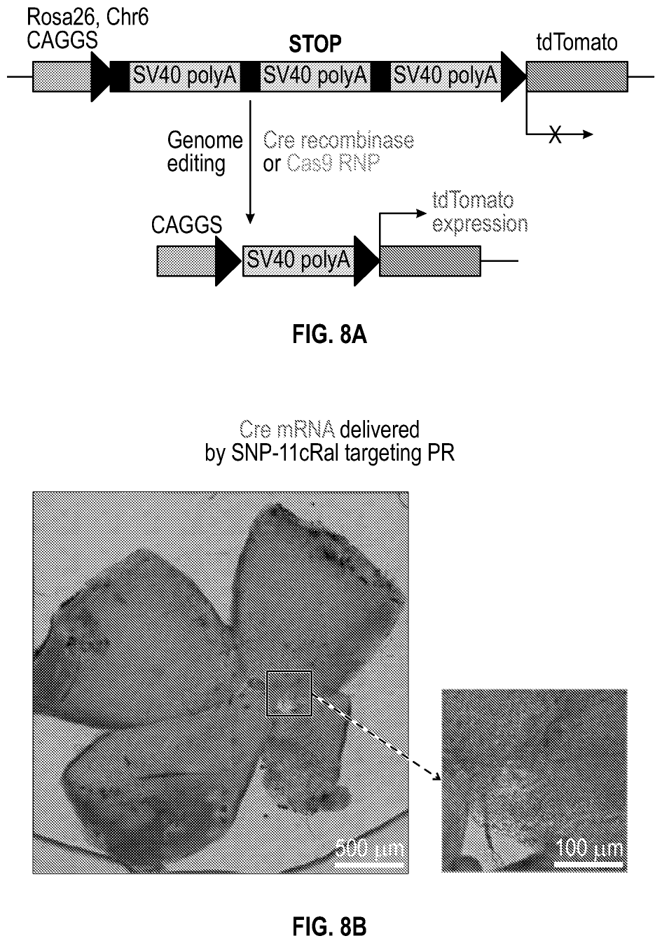

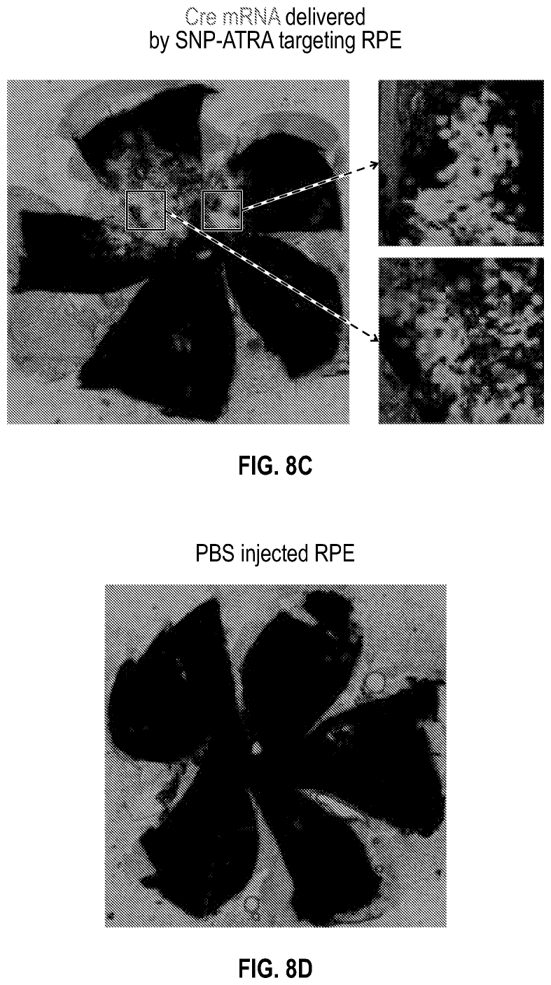

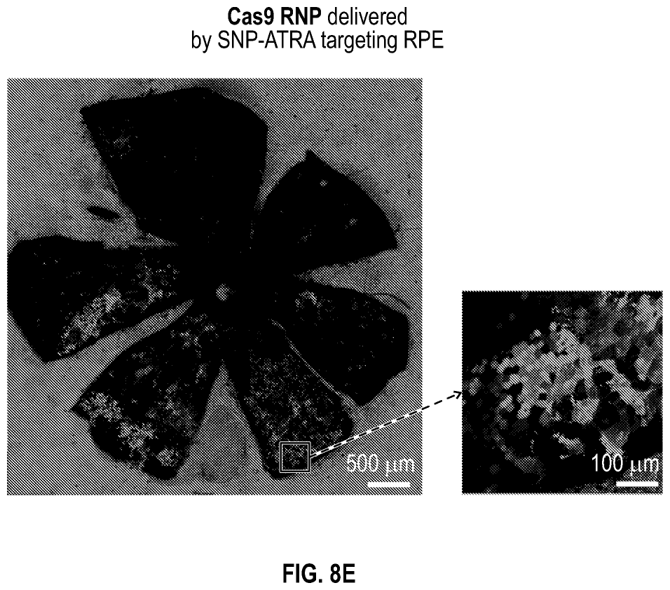

SNPs are suitable for in vivo nucleic acid delivery due to their small size, versatile surface modification, and excellent biocompatibility. The genome-editing efficiency of subretinally injected RNP-loaded SNPs was evaluated in vivo in transgenic Ai14 mice (FIGS. 8A-8E). The Ai14 mice harbor a LoxP-flanked stop cassette (3 repeats of the Sv40-polyA sequence) that prevents the expression of the tdTomato fluorescent protein. CRISPR RNP-guided excision or Cre-lox recombination of the stop cassette leads to tdTomato expression (FIG. 8A). Thus, the Ai14 mouse model provides a robust, high-throughput, quantitative readout of site-specific genome modification at the LoxP-flanked stop cassette locus with a gain-of-function fluorescent signal in cells edited by either Cre-lox recombination via the delivery of Cre-encoding DNA/mRNA, or RNPs targeting the excision of Sv40-polyA blocks (60, 61). Cre mRNA was loaded in SNP-ATRA (for RPE cell targeting) and SNP-11cRal (for PR targeting), respectively, and injected subretinally. Four days post-injection, the RPE layer, and retina layer were then separated and flat-mounted, and the tdTomato expression was evaluated by CLSM. The retina layer was imaged with the PR side up, and this plane included 99% of PRs.

SNP-ATRA induced robust tdTomato expression in the RPE (FIG. 8C). tdTomato expression induced by SNP-11cRal can also be detected in the retina layer (FIG. 8B). These data demonstrated the successful delivery of Cre mRNA by SNPs leading to Cre expression. Moreover, as shown in FIG. 8E, strong tdTomato surrounding the injection site were also observed in RPE cells 12 days post-injection, indicating high gene editing efficiencies enabled by the delivery of Cas9 RNP via ATRA-conjugated SNPs.

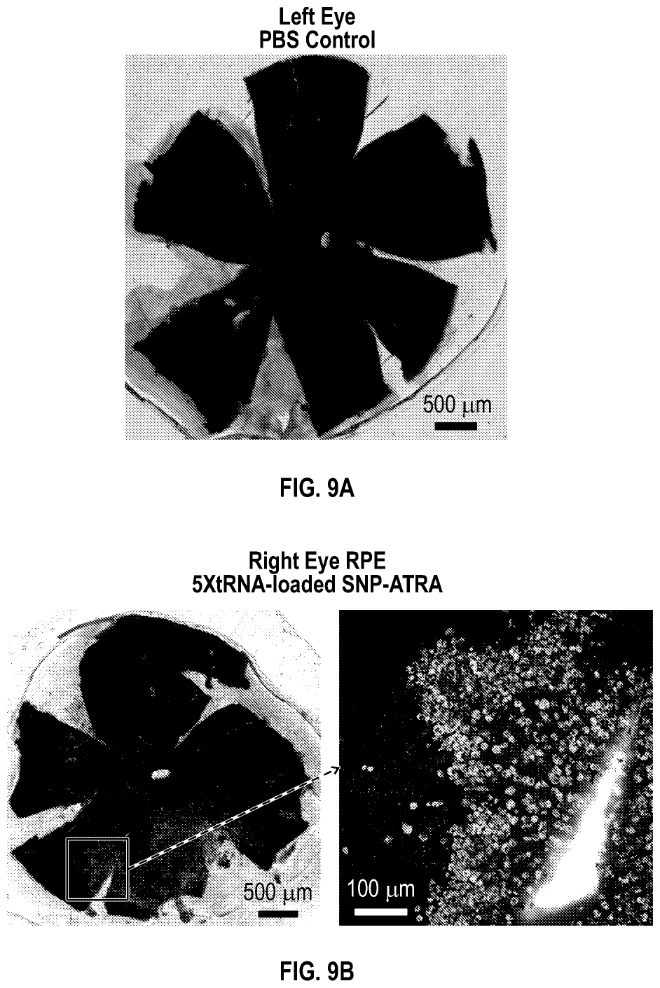

A cDNA plasmid containing five copies of an Arg-UGA ace-tRNA suppressor as well as a fluorescent reporter eGFPY152X (TGA) construct (i.e., 5×tRNA) was loaded into SNP-ATRA, and injected subretinally (FIGS. 9A-9B). GFP expression was evaluated four days post-injection.

As shown in (FIG. 9B), SNP-ATRA induced GFP expression in the RPE, indicating the efficient delivery and subsequent suppression activity of 5×tRNA into RPE cells which then repair the GFPY152X construct.

Cargo Delivery Efficiency of SNP Tested in iPSC-RPE Cells

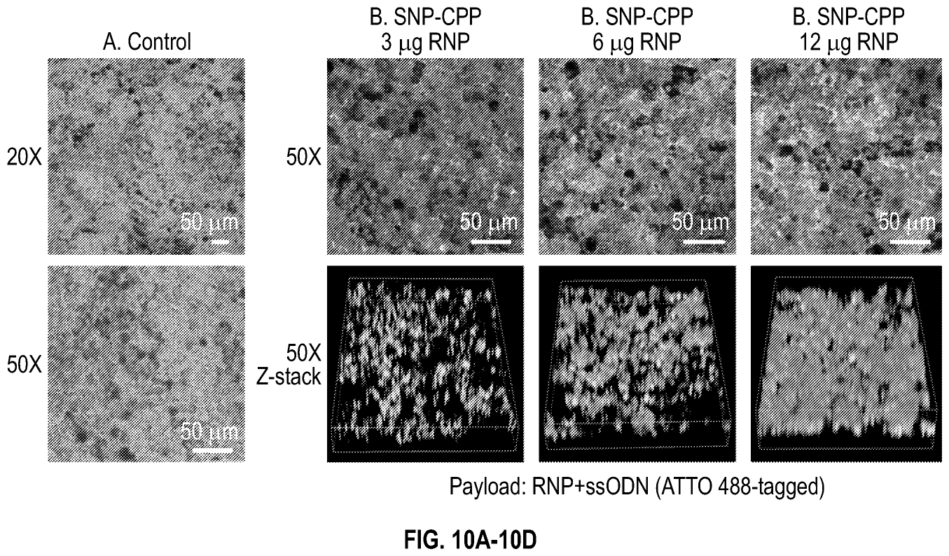

iPSC-RPE is a promising alternative to human RPE for genetic studies, and it has been shown to display identical characteristics of mature human RPE (62, 63). The delivery efficiency of SNPs was also tested in iPSC-RPE cells (FIGS. 10A-10D). RNP with a donor ssODN (RNP+ssODN) with a 1:1 molar ratio was loaded into SNP-CPP (TAT). The donor sequence, ssODN, was tagged with a green fluorescence dye, ATTO-488. iPSC-RPE was treated with RNP+ssODN-loaded SNP-CPP at different dosages, and the cellular uptake of the payload was evaluated four days post-treatment by confocal laser scanning microscopy (CLSM). Significant cellular uptake in iPSC-RPE was observed, and the uptake efficiency was dose-dependent (FIG. 10B-D). In addition, no signs of cytotoxicity were observed, indicating that SNP is an efficient and biocompatible nanoplatform for RNP+ssODN delivery into iPSC-RPE.

Patient-Derived iPSC-RPE as Cellular Testbeds for Therapeutic Development

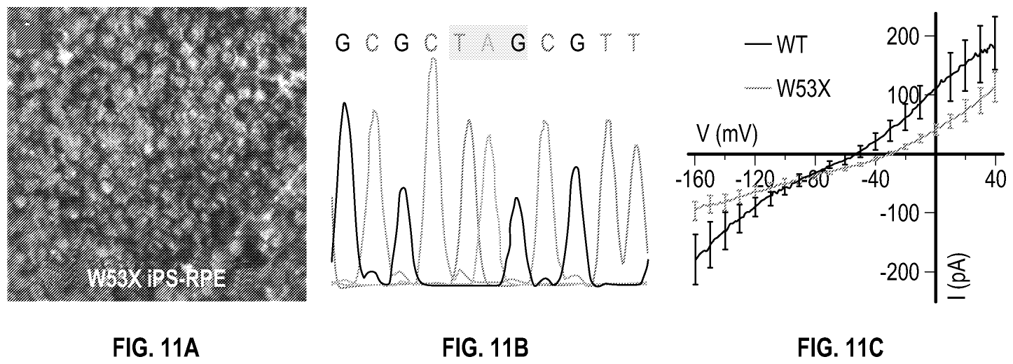

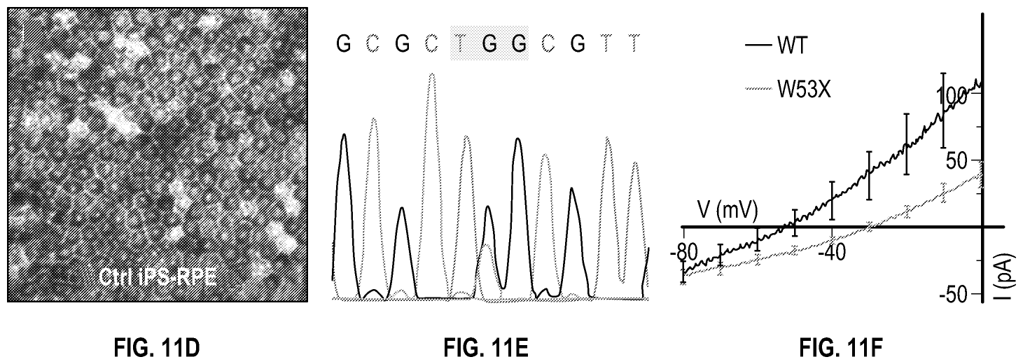

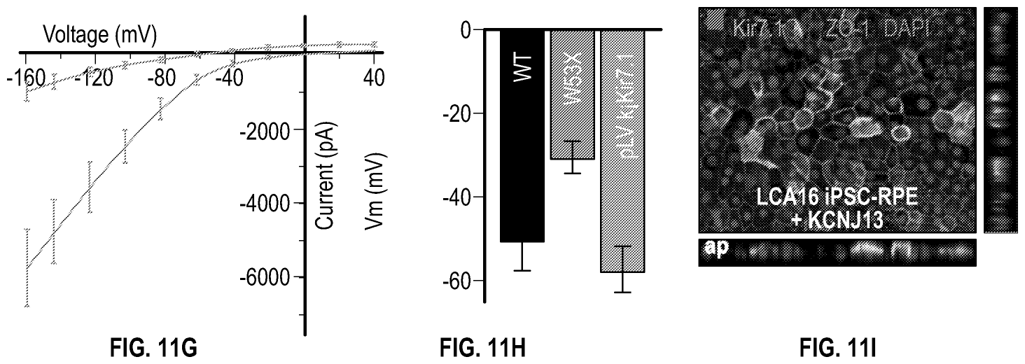

The study of mouse models has made important contributions to our understanding of human retinal disease (64). There have been several attempts to generate mouse models of Kcnj13 knockout, but the mutation results in P0 lethality (35, 65). hiPSC lines have been derived from an LCA-16 patient with the W53X mutation (identified as iPSC-RPEW53X), one unaffected family member (defined as iPSC-RPEKir7.1), and another from an unrelated healthy donor (iPSC-RPEWT) reprogrammed via a lentiviral-mediated transfer of pluripotency factors (FIGS. 11A-11I) (8). The iPSC-RPEW53X cells had comparable karyotype, morphology, expression of RPE cell molecular markers except for Kir7.1 protein expression as confirmed by western blot and immunohistochemistry (FIGS. 11A, 11B, 11D, 11E).

When iPSC-RPE cells are grown on cell culture inserts and were enzymatically digested using papain, pigmented, polarized cells with distinct apical process extensions were obtained. Whole-cell recordings from individual cells revealed a current-voltage (I-V) curve consisting of both inward and outward K⁺ currents, a resting membrane potential of −55 mV, and >10-fold increase in Rb⁺ current (FIGS. 11C, 11F), which is in sharp contrast to the recording from iPSC-RPEW53X cells that showed spontaneous depolarization of at least 15 mV and no increase in current by Rb⁺ (FIGS. 11C, 11F). Kir7.1 channels have a unique permeation for Rb⁺ that increases inward current by more than 10-fold. The results are consistent with a previous observation that the truncated protein product does not form Kir7.1 channels and that Kir7.1 current recording is a reliable measure of the LCA-16 disease state using iPSC-RPE cells as a model system.

Gene therapy through transduction of LCA16 patient-derived iPSC-RPE cells using lentiviral particles carrying GFP-fused human KCNJ13 has also been tested. Open Reading Frame under the control of EF1a and VMD2 promoters. Normal Kir7.1 current in LCA16 iPSC cells after transduction (FIGS. 11G-11I) were recorded. Ion channel protein expression was found to be polarized (FIG. 11I) and at a normal level (FIGS. 11G, 11H).

### Restoration of Current in LCA16 Kir Channel PTC Mutation by Ace-tRNA

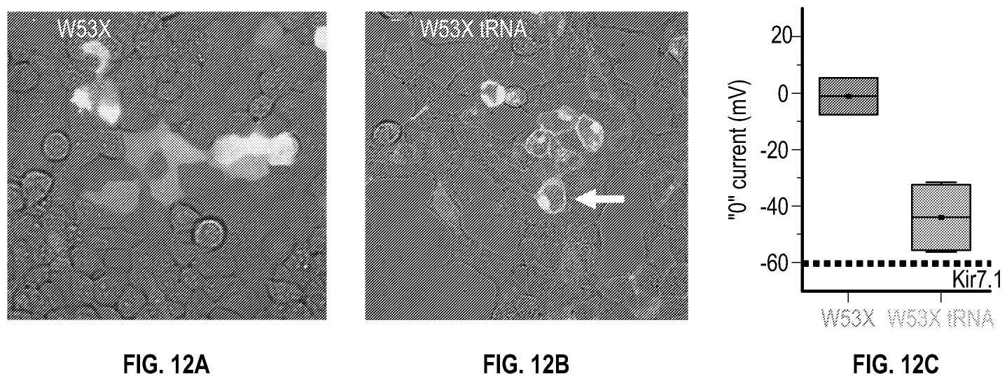

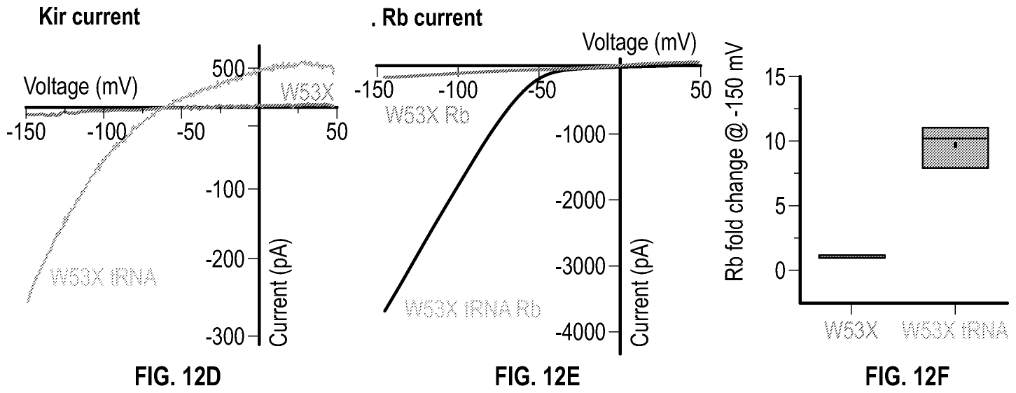

As stated earlier, LCA16 nonsense mutations W53X and R166X results in the non-functional channel. The validity of the proposed ace-tRNA therapy was tested by using transfected HEK293 cells with either GFP fused W53X alone or along with codon edited ace-tRNA^{Trp}. W53X expressing cells showed GFP localization in the cytoplasm, but ace-tRNA^{Trp} induced GFP to be primarily localized in the plasma membrane, the site of Kir7.1 protein expression (FIGS. 12A, 121B). Similarly, whole-cell current recording showed a prominent inwardly rectifying Kir7.1 channel (FIGS. 12C, 2D) compared to a non-functional linear I-V plot (FIGS. 12C, 12D) for cells not expressing tRNA^{Trp}. Compared to −7 and −62 m V membrane potential for W53X and wildtype Kir7.1 channel expressing cells (FIG. 12D), respectively, ace-tRNA^{Trp} treated W53X channel recovered to −53 mV. Proof of channel function rescue by ace-tRNA^{Trp} was also revealed from an almost tenfold increase in inward current using Rb as a charge carrier through Kir7.1 channel (FIGS. 12E, 12F).

Retinal Organoid Production and Photoreceptor Differentiation from Human Pluripotent Stem Cells (hPSCs) and Subsequent Transduction with AAV Vectors

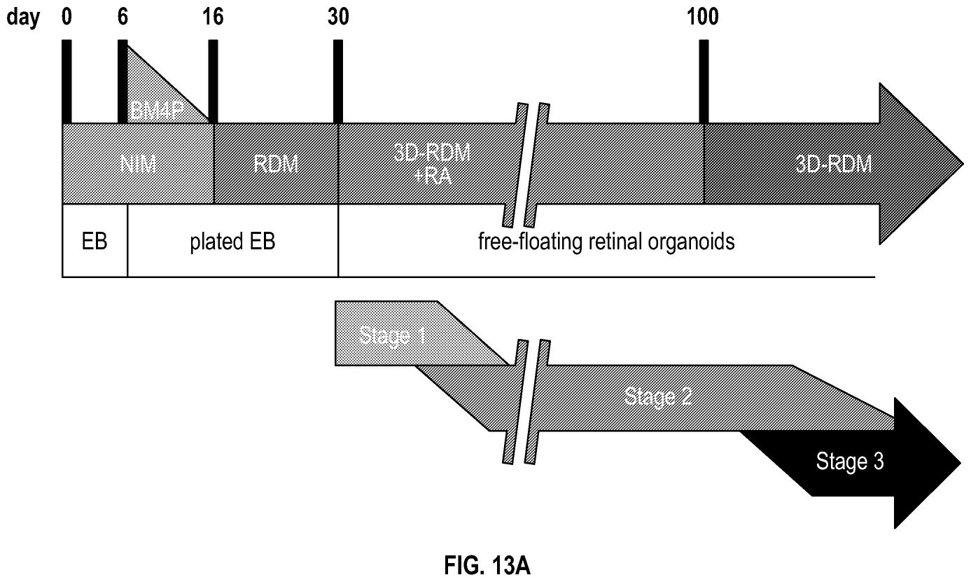

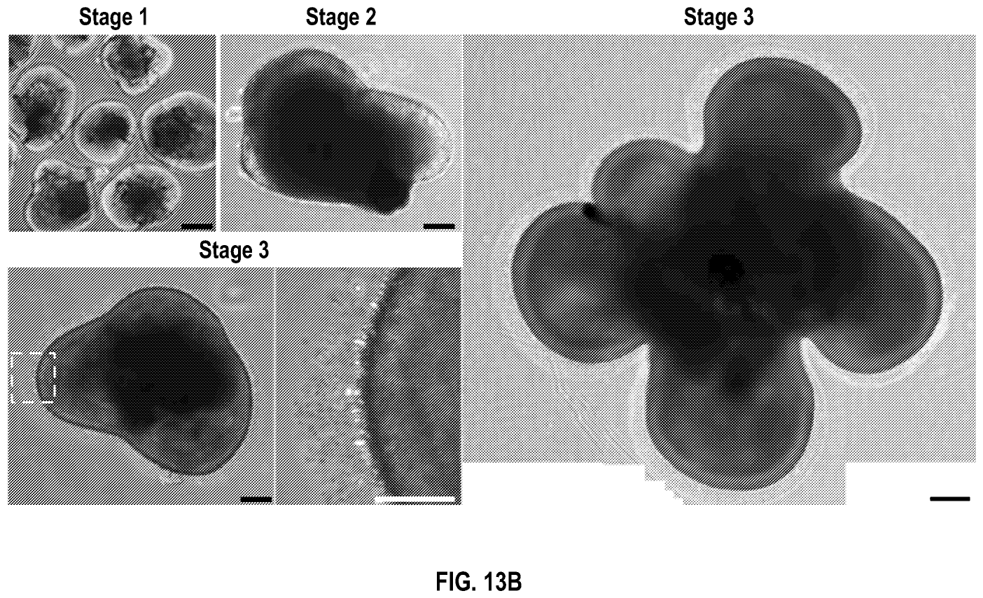

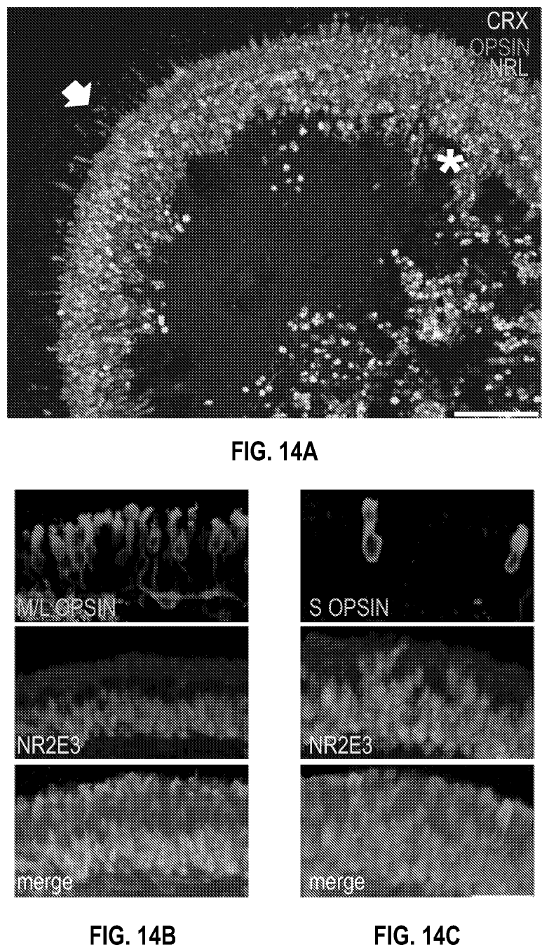

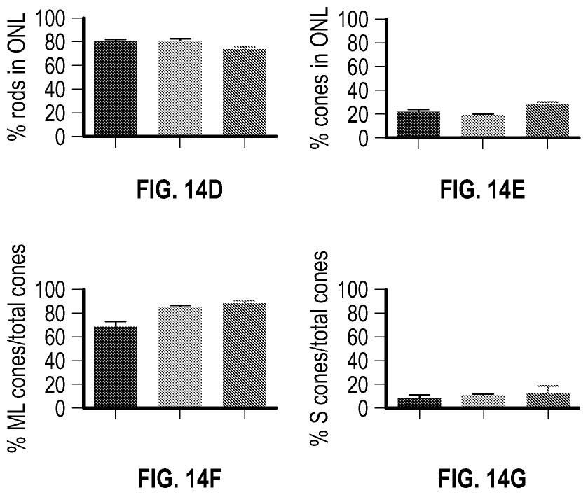

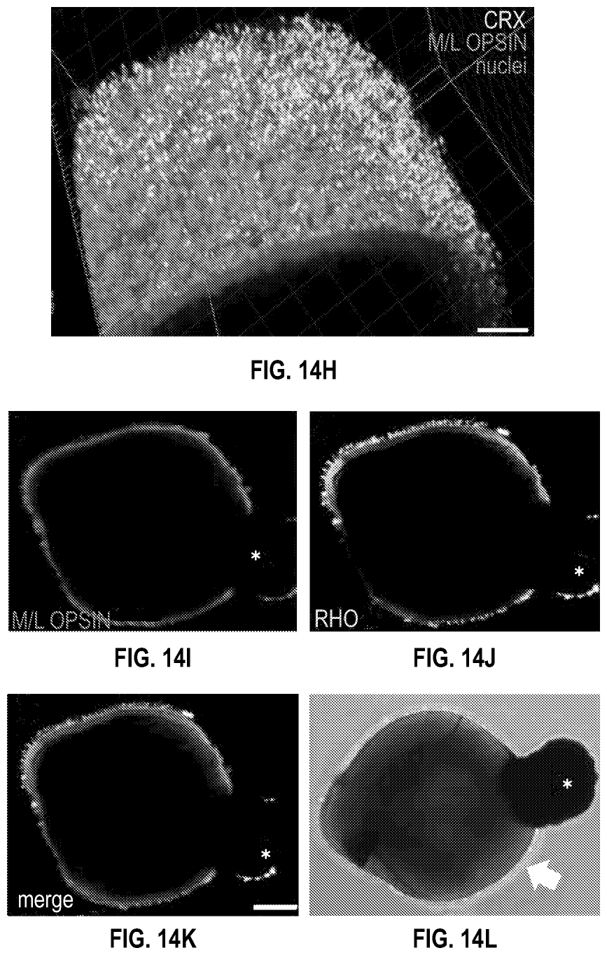

Retinal cell differentiation from hPSCs can use a previously established protocol (2-5) (66-68), with recent modifications to greatly improve production and long-term maintenance of an outer nuclear (i.e., photoreceptor) layer (ONL) from hPSC-OVs (1) (FIGS. 13A-13B). Briefly, hPSCs will be lifted with ReLeSR (STEMCELL Technologies) and grown for 6 days in the neural induction medium (NIM). On day 6 (D6), the suspended cell aggregates are treated with 1.5 nM BMP4 and one day later are reattached to Matrigel-coated plates in BMP4-containing media. The BMP4 is then gradually diluted by half media changes every three days until D16 when cultures are switched to retinal differentiation medium (RDM). Between D27-35, optic vesicle-like neural retina structures (OVs) become clearly visible and are dissected into 3D-RDM, which additionally contains 5% FBS, taurine, and chemically defined lipids (Thermo Fisher) to enhance survival and retinal differentiation. Thereafter, the hPSC-OVs will be maintained in 3D-RDM+retinoic acid until D100, at which time retinoic acid is removed to enhance outer segment formation. The resulting retinal organoids are further differentiated in 3D-RDM until ≥D180, a time point when a well-developed ONL and maturing rod and cone photoreceptors with outer segments are clearly present (FIGS. 14A-14L). Differentiated hPSC retinal organoids then routinely undergo gene and protein expression profiling via PCR and immunocytochemical analyses to verify retinal identity and differentiation state (1-6).

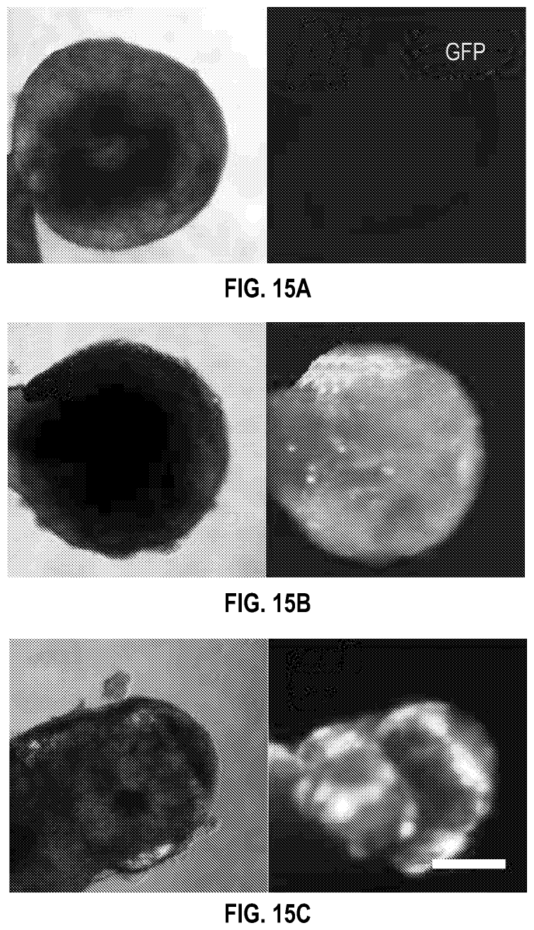

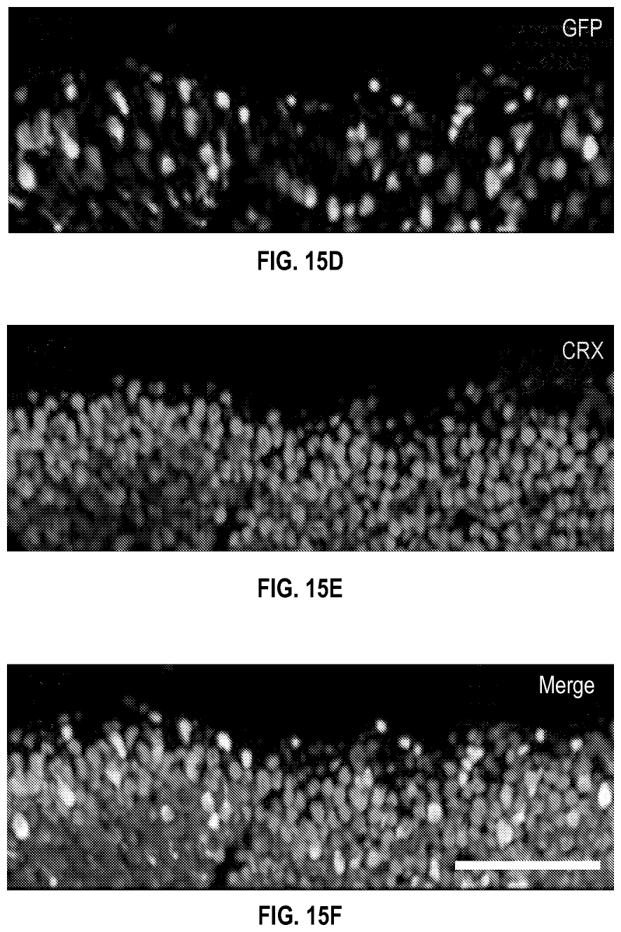

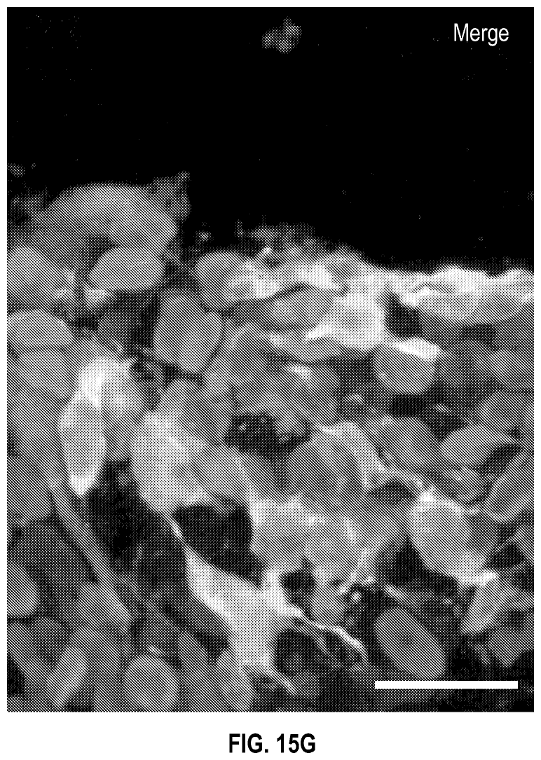

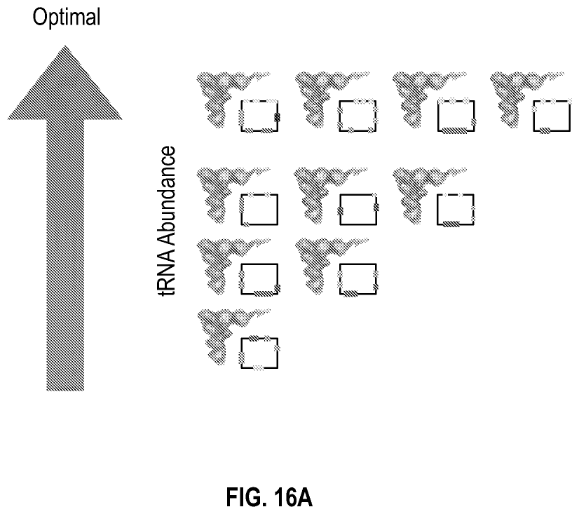

For AAV experiments, hPSC-derived retinal organoids (ROs) in RDM are transduced with 1.5×10¹¹-2.5×10¹² VP/mL AAV with one-half media change 24 hours post-transduction. Thereafter, cultures will undergo daily complete media changes. Based on preliminary AAV-GFP experiments (FIGS. 15A-15G), transgene expression is expected beginning seven days post-transduction, reaching a maximal expression level at approximately 12 days post-transduction. To date, superior transduction of hPSC-ROs using AAV7m8 and AAV5 has been observed (FIGS. 15A-15G).

### AAV2.7m8 Mediated Transduction of Cells as Well as Posterior Retina Through Intravitreal Injection

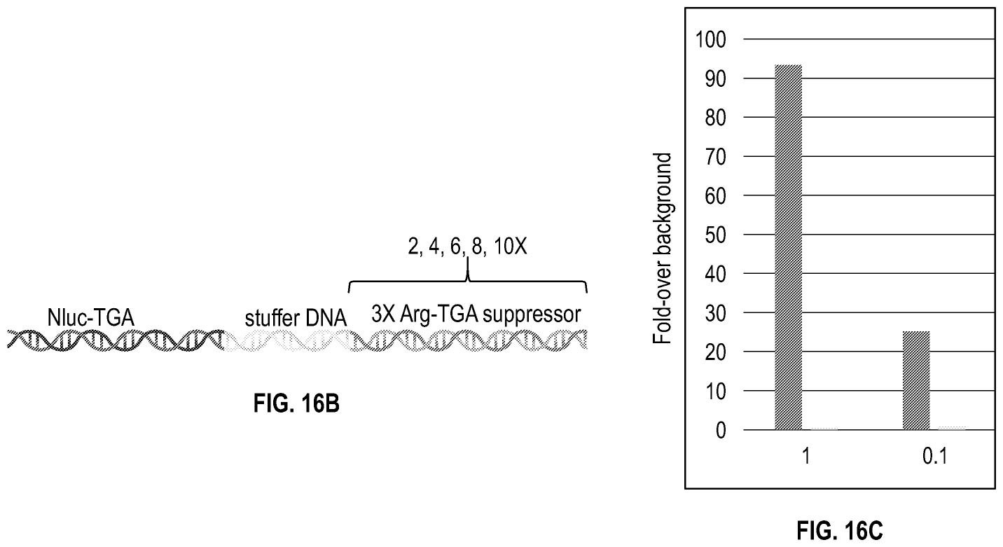

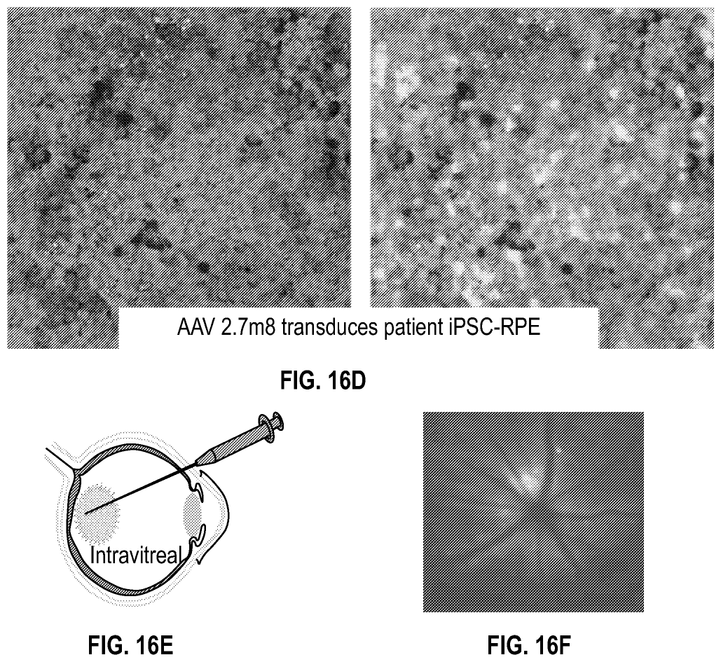

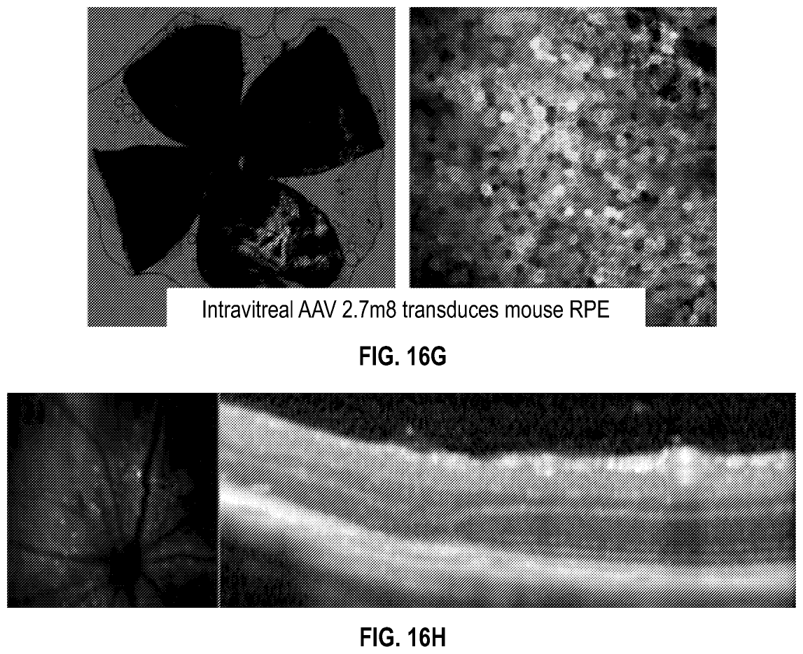

To demonstrate AAV mediated gene transduction (FIGS. 16A-16I), AAV packaging of ace-tRNA (FIG. 16A, 16B) was used. The initial test using a lentiviral construct showed a higher expression of ace-tRNA in an iPSC neuron model (FIG. 16C). Then use of 1 μl of 1×10¹¹ rAAV2.7m8 viral titer per 6 mm transwell showed GFP expression in every iPSC-RPE cell in the field without compromising cell health (FIG. 16D). The same rAAV2.7m8 vector was delivered to the mouse eye by intravitreal injection (2 μl of 1×10¹¹ viral vector) (FIG. 16E). After eight days, GFP expression in the posterior retina was imaged by fundus autofluorescence imaging (FIG. 16F), and the OCT image of the retina appeared normal (FIG. 16H). Examination of flat-mount of RPE cells showed GFP expression in RPE cells, and the morphology of the RPE cells appeared normal cobblestone shaped. This result indicates that there is lower cellular toxicity and higher transduction expression of GFP in vivo through the vitreal delivery of AAV2.7m8.

### Generation of Nonsense Mutant Clones

Site-directed mutagenesis has been performed to generate both W53X and R166X clones of the human KCNJ13 gene (8, 29) and made constitutively expressing HEK-FRT cell lines using Flip-In system (Thermo Fisher). Human mutant clones of BEST1^{R200}, CACNA1F^{R680} and KCNV2^{E148} can be used in mammalian expression plasmids. These nonsense mutant clones can be expressed in HEK293T cells, and respective protein functions quantitated by whole-cell patch-clamp electrophysiology in comparison with wildtype clone (positive control), non-treated cells, and mock-transfected cells (negative control, using empty plasmid). Experiments can be repeated at least six times to determine the statistical difference between groups.

For the experimental group, respective ace-tRNA constructs (tRNA^{Trp}, tRNE^{Arg}, and tRNA^{Glu}) along with the mutant clones are packaged in silica nanocomplexes for delivery to HEK cells. Two days after transfection, electrophysiology can determine the functional rescue of channels after ace-tRNA delivery. Full-length protein expression can be confirmed by immunolocalization and Western blot determination using respective antibodies. In the case of KCNJ13 W53X and R166X HEK-FRT cell lines, ace-tRNA^{Trp} and ace-tRNE^{Arg} packaged in silica nanocomplex can be used, followed by electrophysiology and expression studies. Results can be compared with cell lines expressing mutant channels or control HEK-FRT cells.

### Production of Minicircles Containing Ace-tRNA ORF

Minicircle episomal DNA vectors are minimized eukaryotic expression cassettes without the prokaryotic plasmid DNA backbone (6, 75). In experiments where equimolar reporter gene expression cassette was used for in vitro experiments, minicircle DNA has demonstrated three to ten-fold higher expression in the absence of cytotoxicity (76) and at least 10-13 times more expression in vivo (77). The smaller size of the DNA plasmid is favored for diffusion across the plasma membrane, its escape of endo/lysosomal degradation, and cross the nuclear membrane for long term gene expression (78, 79). The plasmid remains in a stable supercoiled structure and can incorporate a cell-specific promoter for cellular targeting to either PR or RPE cells. Minicircle technology has seen widespread use in the stem cell field for reprogramming because of the transgene-free expression of factors and in inherited retinopathy (80, 81). Minicircles are well-suited for the expression of tRNA because the encoding region for an individual tRNA is ˜75 bases in length, and the promotors for tRNA expression (the A and B box regions) are contained within this 75 base sequence. The minimal ‘expression cassette’ for ace-tRNA expression has been found to also includes a ˜70 base 5′ genomic sequence upstream of the tRNA and a short (6 base) ‘terminator’ sequence 3′ to the tRNA sequence. Thus, ˜150 bases represent the entire genetic package.

The coding sequence for the promoter (for the reporter gene) and ace-tRNA can be subcloned into a parental plasmid (System Biosciences, Palo Alto, CA). MC-Easy™ (Cat #MN920A-1, SBI System Biosciences) Minicircle DNA production kit can be used to obtain high-quality Minicircle DNA. Purified bacteria-free MinicircleDNA can be packaged in SNP for delivery to iPSC cells or subretinal injections.

AAV Packaging and Delivery of tRNA ORF

AAV2.7m8 can be used for outer retina transduction. In addition, existing AAV systems display a maximal ˜4.5 kb ‘payload’ amount, which limits the size of the gene that can be delivered to the retinal cell. Transfer RNA is compact, ˜75 bases, and requires limited surrounding sequence for biological processing: a 5′ short ˜100 base genomic ‘leader sequence’ and a 3′ 6-base termination sequence.

The coding sequence of the ace-tRNA (leader and terminator) can be packaged within the AAV payload region between ITR sequences. The tRNA ‘cassettes’ can be interspersed by non-identical cDNA sequences which serve to minimize recombination. For NHP experiments, the AAV can either have a payload of VMD2-GFP^{Arg}-ace-tRNA^{Arg} for RPE cell expression or hGRK1-GFP^{Arg}-acetRNA^{Arg} for expression in the PR. High-titer (10\hat{ }14 genome copies per milliliter) affinity-purified ready to use AAV2.7m8 can have specificity for either PR (OPSIN) or RPE (VMD2) based on the choice of cell type-specific promoter. A minimized VMD2 promoter, and optimized intravitreal delivery to avoid toxicity have been used (83). The transgene is optimized for expression in hiPSC-RPE and in vivo mouse RPE. Also, a T2A-GFP can be included so that whole-cell electrophysiology analyses of transduced cells can be individually performed. AAV5 also transduces PR in hiPSC-organoids and mouse RPE cells, providing an alternative approach for expressing ace-tRNA.

### Design, Synthesize, and Optimize Intravitreally Injected SNPs for PR- or RPE-Targeted Ace-tRNA Minicircle Delivery

Intravitreally injected SNPs targeting either PR or RPE can be engineered and optimized for tRNA minicircle delivery. The SNPs coated with HA and conjugated with various targeting ligands in HEK293, iPSC-RPE, iPSC-PR cells can be first tested in vitro.

Design and Synthesize SNPs for tRNA Minicircle Delivery

Subretinally injected SNPs can efficiently deliver a number of payloads to RPE and PR cells in vivo, but intravitreal delivery is less invasive. Functionalizing the surface of the SNPs with hyaluronic acid (SNP-HA) can not only promote retinal penetration but also enhance the cellular uptake of the SNPs by PR or RPE cells via receptor-mediated endocytosis, making it a desirable nanovector for intravitreal delivery of nucleic acids in PR and RPE cells. In addition, both 11cRal and ATRA have a high binding affinity to interphotoreceptor retinoid-binding protein (IRBP) (90, 91). IRBP is a major protein in the interphotoreceptor matrix (IPM) that selectively transports 11cRal to PR, and ATRA to RPE cells (58, 59, 92). Therefore, SNP-HA conjugated with 11cRal and ATRA can further increase the transfection efficiency and specificity of ace-tRNA minicircles into PR and RPE cells, respectively, through such a selective transport mechanism.

HA-modified SNPs can be engineered for intravitreal delivery of ace-tRNA minicircle, or co-delivery of ace-tRNA minicircle and a GFP plasmid as a reporter system for initial formulation optimization. Furthermore, the potential synergistic effects of hybrid targeting ligands can be investigated (e.g., HA in combination with either 11cRal (for PR) or ATRA (for RPE cells)). With SNPs that are small (e.g., 40-50 nm) and coated with hyaluronic acid, it is expected that the depth of penetration and area of diffusion will be higher than other nucleic acid nanocarriers (55, 87-89, 93).

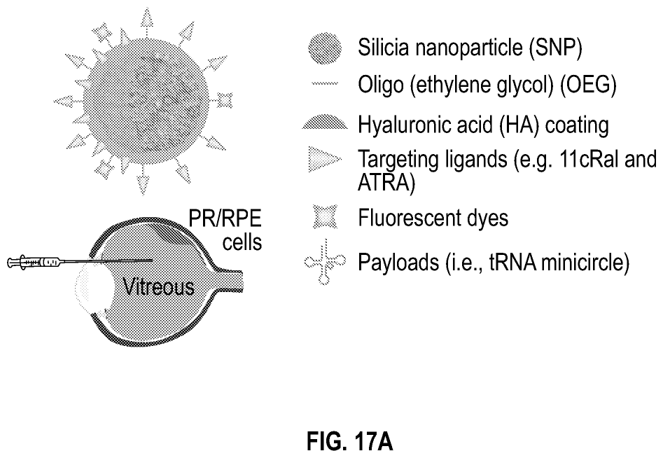

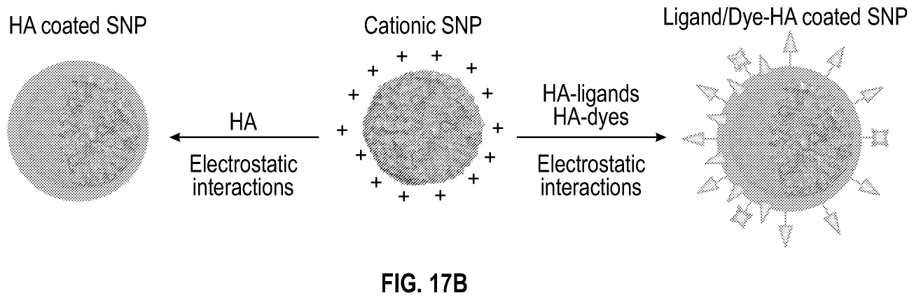

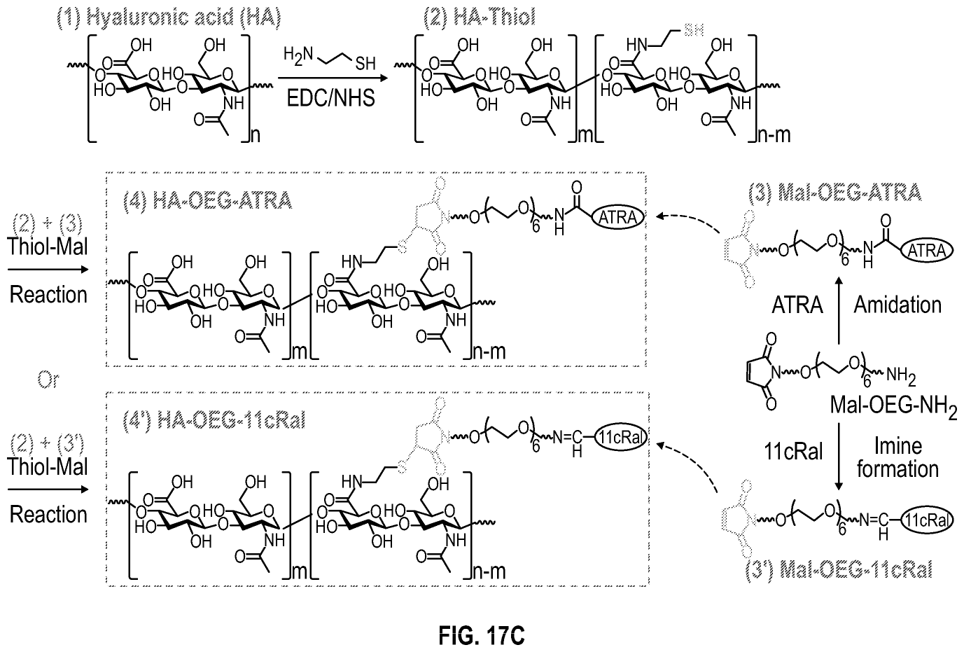

FIG. 17A shows the SNPs re-engineered for intravitreal injections. SNP-HA, as well as 11cRal-conjugated SNP-HA (i.e., SNP-HA-11cRal targeting PR), and ATRA-conjugated SNP-HA (i.e., SNP-HA-ATRA targeting RPE) are prepared. 3-Aminopropyl)triethoxysilane (APTES), a cationic silane monomer, can be used as an additional reactant during the fabrication of the SNPs via the microemulsion process to alter the surface charge of the pristine SNPs (i.e., without PEGylation) from anionic to cationic. The size of SNPs will not be influenced significantly as it is controlled by the water-in-oil microemulsion process. As presented in FIG. 17B, after purification, the cationic SNPs can be coated with either HA (an anionic polysaccharide) or ligand-conjugated HA, at a ratio of HA saccharide unit/APTES=10, via electrostatic interactions. Uncoated HA can be easily removed by washing and centrifugation.

To conjugate targeting ligands on HA, as shown in FIG. 17C, the HA will first react with 2-aminoethanethiol through amidation to yield HA-Thiol. Meanwhile, targeting ligand 11cRal can be conjugated to NH₂-oligo (ethyleneglycol) (OEG)-Maleimide (Mal) through imine bond formation, while ATRA can be conjugated to NH₂-OEG-Mal through amidation. Thereafter, the Mal-OEG-11cRal/ATRA can be conjugated to HA-Thiol through a Michael-addition reaction to form HA-OEG-11cRal/ATRA. The chemical structure of all intermediate and final polymer products can be characterized by nuclear magnetic resonance spectroscopy (NMR). HA with a low molecular weight (i.e., 10 kDa) as it can lead to thinner HA coating to yield smaller SNP-HA sizes and provide higher reactivity for ligand conjugations will be used. OEG₅₀₀ as the spacer for ligand conjugation will be used as it may enhance the binding efficiency of the ligand-conjugated SNPs to IRBP.

Since the negatively charged surface might affect cellular uptake, the molar ratio of HA saccharide unit/APTES is optimized (i.e., 5, 10, and 15). The amounts of targeting ligands on HA and the molecular weight of HA and OEG can be optimized both in vitro and in vivo. The amounts of the targeting ligands can be controlled by the substitution degree of ligands on HA. The different molecular weights of HA and OEG can be directly ordered from Lifecore (Chaska, MN). The substitution degrees of the HA-OEG-11cRal, or HA-OEG-ATRA can vary from 0, 10, 20, 30, to 40% (0% means unmodified HA). The molecular weight of HA can vary from 10 kDa to 1 MDa. The molecular weight of OEG can also vary from 200 Da to 1 kDa. A family of SNPs coated with different types/substitution degrees of ligand-modified HA can be prepared. Their morphologies and zeta-potentials can be characterized by TEM and DLS.

In a study, cationic silane monomer (i.e., APTES) at a 10% molar ratio was successfully incorporated into SNP and yielded a positively charged SNP (with zeta-potential at +25 mV) with efficient nucleic acid delivery in HEK 293T cells. The HA coating process and parameters will be optimized by varying (1) the HA/SNP ratio, (2) HA molecular weight, and (3) the ligand conjugation ratio to HA.

In terms of targeting ligands, other than employing 11cRal/ATRA for targeting PR/RPE cells, other types of targeting ligands can be explored to optimize the delivery efficacy in vivo (for example, aptamer (AS1411)). AS1411 is a G-quartet DNA aptamer that targets nucleolin, which is an RNA and protein-binding protein expressed on the surface of PR and RPE cells (94). AS1444 can facilitate the transport of the SNPs to PR/RPE cells and enhance their uptake by PR/RPE cells. To conjugate this aptamer onto the SNPs, a thiol group on its 5′-end (Integrated DNA Technologies, IA) can be used, which can then be linked to HA-OEG-Mal through a Michael-addition reaction. The resulting HA-OEG-aptamer can be used to coat the SNPs through electrostatic interactions to yield SNP-HA-aptamer. The AS1411 density (e.g., 10, 20 and 30% in molar ratio to HA saccharide unit) can be controlled by varying the feed molar ratio of AS1411 and HA-OEG-Mal during the reaction.

### Evaluate the Cellular Uptake, Biocompatibility, and Transfection Efficiency of Non-Viral Ace-tRNA Minicircle Delivery SNPs In Vitro

The cellular uptake, biocompatibility, and transfection efficiency of the SNPs engineered for the delivery of ace-tRNA minicircle can be evaluated in vitro using human embryonic kidney cells (HEK 293T cells) first and then induced pluripotent stem cell-derived RPE cells (iPSC-RPE) and iPSC-derived PRs (iPSC-PR), respectively. For cellular uptake studies, the SNPs can be conjugated with a fluorescent dye (e.g., Alexa Fluor 488 or 594) via OEG conjugation. Both flow cytometry and fluorescence microscopy can be used to monitor the cellular uptake at various time points. The biocompatibility of the SNPs can be evaluated using a colorimetric MTT assay. To facilitate the initial evaluation and optimization of the SNPs, both ace-tRNA minicircle and GFP-encoding plasmid DNA (at 1:1 molar ratio) in the SNPs can be encapsulated. Successful transfection leads to GFP expression; thus, it will serve as a fluorescence reporter system for SNP formulation optimization. Using this reporter system, the in vitro delivery efficacy of SNPs can be observed by CLSM and quantified by flow cytometry by counting GFP-positive cells. However, for the majority of in vitro and subsequent in vivo studies where cells and animals with target mRNA present, SNPs can be used to deliver ace-RNA minicircle alone. In this case, editing efficiency can be measured through deep sequencing. Meanwhile, full-length protein expression and electrophysiological studies can be done with treated cells to assess ACE-tRNA minicircle mediated functional restoration of ion channels.

The following SNP formulations can be tested: (1) in HEK 293T cells, cationic SNP (with different cationic APTES ratio, namely, 10, 20 and 30% in a molar ratio to the total silica reagents (i.e., TEOS and BTPD)), and SNP-HA (with different HA/APTES molar ratios (e.g., 5, 10, and 15), and HA molecular weights ranging from 10 kDa to 1 MDa). (2) In iPSC-RPE, SNP-HA (without ligands) and SNP-HA-ATRA (with 10, 20, and 30% in a molar ratio to HA saccharide unit). (3) In iPSC-PR, SNP-HA (without ligands) and SNP-HA-11cRal (with 10, 20, and 30% in a molar ratio to HA saccharide unit). For the control groups to be tested in all three cell types, SNPs with untreated cells, Lipofectamine 2000-treated cells (with payload), and SNP formulations loaded with negative control payloads (e.g., non-coding nucleic acids) can be compared.

### In Vitro Transfection Method

HEK293T cells can be cultured on 24-well plates. SNP formulations can be treated with cells with dosages varying from 3-12 ug payload per well. ace-tRNA minicircle delivery efficiency can be analyzed four days post-treatment. Similarly, iPSC-RPE and PR experiments and assessment of study outcomes can be evaluated.

### Optimize the SNP Formulations for ACE-tRNA Minicircle Delivery in PR and RPE Cells In Vivo Through Intravitreal Injections

SNPs can encapsulate ace-tRNA minicircles. For optimization, the ligand (i.e., ATRA and 11cRal) can be conjugated to HA at 10, 20, and 30% in a molar ratio to the HA saccharide units, prior to coating to SNPs. HA with varying molecular weights, including 10, 100, and 1000 kDa, can be tested. The OEG length for ligand conjugations to HA can be kept at 500 Da initially. SNP-HA without any ligands can be used as one of the controls. Several controls other than non-ligand SNPs can be included in the studies. In particular, the delivery efficiencies of the aforementioned SNP formulations with PBS, naked ace-tRNA minicircle, Lipofectamine+ace-ERNA minicircle, non-coding ace-tRNA, and ultimately the optimal SNPs with coding Ace-ERNA minicircle can be compared. The SNP formulations encapsulating ace-tRNA minicircles and the controls at 8-16 ug (2 μl solution by intra-vitreal delivery) can be injected in Rd1, and Rd12 mice disease (RPE/PR) models.

To assess the ace-tRNA minicircle delivery efficiency by imaging (ace-tRNA formulations along with a reporter gene), the mice are euthanized, and eyes collected four days after injection. Enucleated eyes from these mice are rinsed twice with PBS, a puncture is made at ora serrata with an 18-gauge needle, and the eyes opened along the corneal incisions. The lens is then carefully removed. The eyecup is flattened, making incisions radially to the center, to give the final ‘starfish’ appearance. The retina separated gently from the RPE layer. The separated RPE and retina are flat mounted on the cover-glass slide and imaged with NIS-Elements using a Nikon C2 confocal microscope (Nikon Instruments Inc.). The ace-tRNA delivery efficacy can be determined by PCR amplification and detection of the ace-tRNA sequence. Any reduction in visual acuity, reduction in the ERG response, and changes in retinal structure in RPE, PR, or inner retina by either mock delivery of SNP alone or SNP containing ace-tRNA minicircle can be indications of toxicity.

Ultrasound (US)-enhanced intravitreal delivery of the various SNP-HAs can also be tested. Briefly, microbubbles (i.e., LUMASON®, an FDA-approved ultrasound enhancing agent) will be injected intravitreally together with the ligand-conjugated SNP-HA formulations. A therapeutic US machine and a 2-cm² probe will be employed. The US exposure parameters are as follows: frequency, 1 MHz; intensity, 2 W/cm²; duty cycle, 50%; pulse recurrent frequency, 100 Hz; duration, 5 min (95).

Further, preliminary data suggest that SNPs conjugated with proper targeting ligands such as 11cRal and ATRA can successfully deliver the ace-tRNA minicircle to the PR and RPE cells in vivo. In addition, cell-penetrating peptides (CPPs) can be used to enhance the delivery of NPs to the PR and RPE cells. Examples of CPPs include TAT, an arginine-rich peptide (97), and POD, a novel peptide used to enhance the DNA delivery efficiency into retinal cells in vivo (98, 99). Since CPPs lack cell-type specificity, they can be used for subretinal injection together with PR- or RPE cell-specific ligands to enhance the specificity for PR or RPE cell-targeted delivery as well as the cellular uptake of the SNPs.

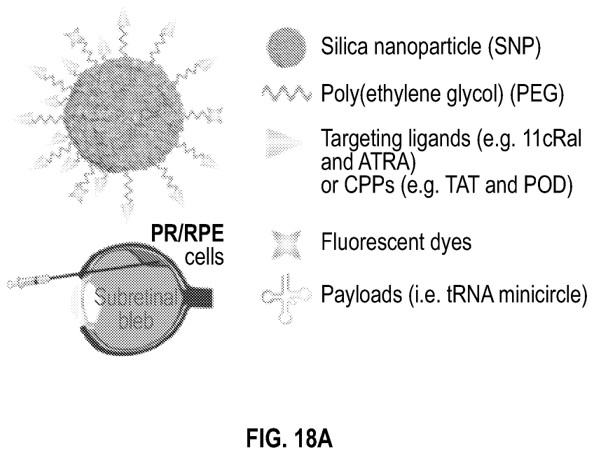

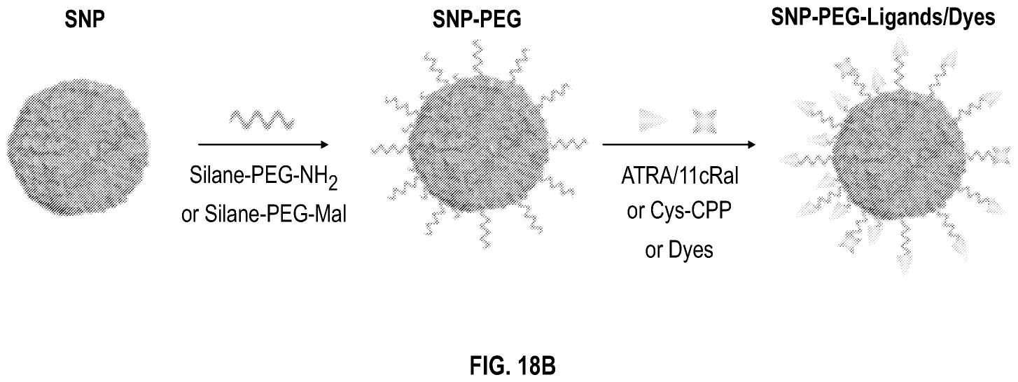

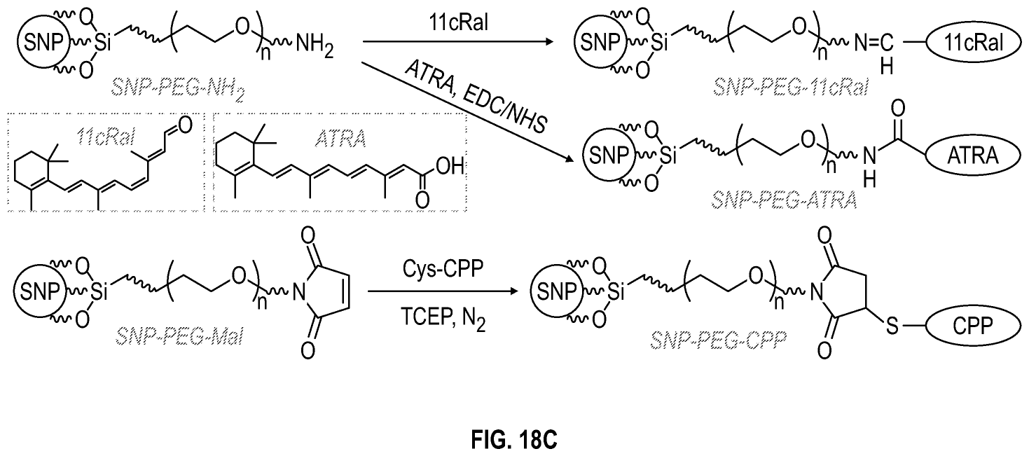

FIGS. 18A-18C presents the design of SNPs for subretinal injections. GSH-responsive SNPs are synthesized, and targeting ligands or CPPs conjugated on SNP surfaces with PEG (SNP-PEG) as the spacers. FIGS. 18B and 18C show the SNP surface modification process and the synthesis schemes of 11cRal- or ATRA-modified SNPs or CPP-modified SNPs. 11cRal or ATRA are conjugated to the PEG terminal ends by imine bonds or via amidation, respectively. CPPs (e.g., TAT and POD) re modified with a terminal thiol group-bearing cysteine, and then conjugated on the SNP-PEG surface via Michael-addition reaction. The morphology and zeta-potential of the SNPs are characterized by TEM and DLS. The subretinally injected SNP formulations with varying PEG density (i.e., PEG/SNP weight ratio) and varying types (11cRal, ATRA, CPPs) and amounts of ligands/CPPs (i.e., the molar ratio of ligands/CPPs to total surface PEG) are evaluated in HEK293T, iPSC-RPE, and iPSC-PR cells. Two factors can be studied to achieve optimal in vivo efficacy, including: (1) the density of PEG on SNP, which can be tuned by varying the weight ratios of PEG in SNP (with three levels, i.e., 1/10, 2/10 and 3/10), and (2) the density of targeting ligands/CPPs on SNP, which can be adjusted by varying the molar ratios of targeting ligands/CPPs to PEG on each SNP (with three levels, i.e., 0, 20, 40 and 60% in a molar ratio to PEG).

Rescue of Phenotype in iPSC-RPE and iPSC-PR Models of Blindness

For monogenic diseases, iPSC technology provides a unique opportunity to create human disease models with a patient-specific genetic signature (19). In undertaking any iPSC-based disease modeling effort, channelopathies were the focus. Standard electrophysiological techniques are used to ascertain the impact of ace-tRNA therapeutics.

Cell Lines and ace-tRNA Delivery. Differentiation of iPSCs to Retinal Organoids and RPE and Subsequent Characterization of Maturing Cultures

Retinal organoids from LCA16 and multiple Best patients have been produced, as well as their gene-corrected isogenic controls (8, 74, 101). For the BEST1^{R200} line, a wildtype iPSC line is gene-edited to generate the appropriate nonsense mutation model in BEST1 (74). Both the CACNA2D4^{R628} and CACNA1F^{R680} iPSC lines can be used. All patient-specific and gene-corrected iPSC lines are differentiated to RPE or stage 3 organoids (which contain an abundance of PRs with outer segments evident by light microscopy) as previously described (8, 67, 74, 100, 101).

### Characterization of the PR Phenotype of Mutant Retinal Organoids and Initiation of Read-Through Drug Experiments

Overall, with the iPSC tools the ability of read-through ace-tRNA drugs to restore the production of full-length protein in differentiated KCNJ13 and BEST1 in iPSC-RPE and CACNA1F and CACNA2D4 in iPSC-PR can be tested. For the iPSC-RPE cells, ace-tRNA is delivered through SNP ATRA minicircle or AAV2.7m8. For the iPSC-PR, ace-tRNA is delivered using SNP 11-Cisretinal minicircle and AAV2.7m8. The expression of ion-channel current and full-length protein expression can be determined in the KCNJ13, BEST1, CACNA1F, and CACNA2D4 cells. Patch-clamp measurements on the enzymatically dissociated cells at single-cell resolution generate a highly accurate real-time electrophysiological measurement of channel activity. Details of the voltage dependence and current response waveforms combined with the specialized composition of salt solution allows for the identification of the activity of a singlechannel type. TotalSeq™ merged protein analysis and scRNA-seq can be used, following ace-tRNA treatment, using specific antibody derived TAG for library construction (Biolegend) (102, 103).

Analysis of whole-cell electrophysiology will find the characteristic ionic current (Kir7.1, Ca2+ activated Cl—, Ca2+) and pharmacological profile (8, 74, 104). Measured channel kinetics such as voltage dependence and pharmacological drug sensitivity can be indicative of minor changes after therapeutic interventions. Previous studies showed a successful recording of both Kir7.1 and Ca2+ activated Cl— currents in KCNJ13 and BEST1 mutant iPSC-RPE after gene therapy (74, 105). Protein expression analyses are expected to show membrane localization of the channel protein and a full-length protein product compared to non-treated cells, as has been previously shown in KCNJ13 and BEST1 mutant iPSC-RPE. TotalSeq™ validation can determine any divergence between RNA and protein expression, and any effect ace-tRNA might have on the global transcriptional profile (74). AAV9 or AAV5 vectors can be considered.

### Restoration of Visual Function in PTC Mouse Models of PR and RPE Dystrophy Rationale

Several mouse mutants are available through the Translational Vision Research Models (TVRMs) with the established molecular basis for the human blindness phenotype for testing drugs targeting either PRs or RPE cells. The rdl mice carry an autosomal recessive natural mutation 347C-T with an mRNA codon transition UAC to UAA in the PR Pde6b gene that causes early-onset severe retinal degeneration (106, 107). AAV-mediated gene therapy has restored the retinal structure and function of rd1 mice (108).

Similarly, Cacna1f G305X loss-of-function mice is a model of human CSNB2A and has been well characterized for electroretinogram and retina morphology (109, 110). The functional restoration of the Cav1.4 channel is a good model for PR targeting of ace-tRNA. nob2 mice have a spontaneous loss-of-function mutation null mutation. For targeting the RPE cells, rd12 mice is a spontaneously arising LCA mice with an R44X mutation in the Rpe65 gene (111). The targeting of RPE cells with ace-tRNA can, therefore, rescue retina morphology, protein expression, and visual function.

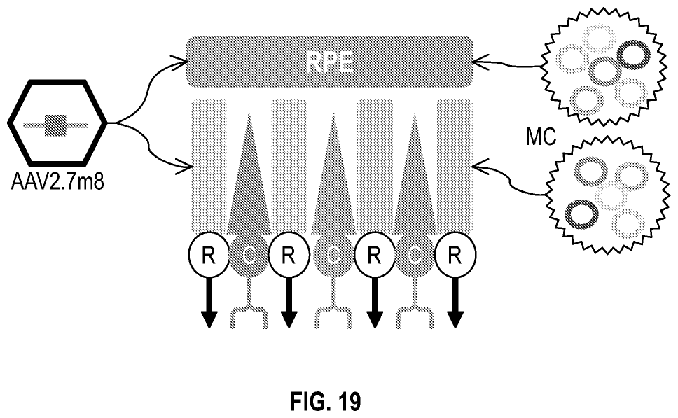

The mice can be bred and maintained. About 2-3 wks old mice are used for intravitreal injections targeting either PR or RPE (FIG. 19) for the following experiments.
1) PR-rdl/Cacna1f eyes receive tRNA through AAV2/9.
2) RPE-rd12 eyes receive tRNA through AAV2.7m8.
3) A Kcnv2 E314X mice colony established through CRISPR genome editing mediated mutation knock-in. These Kcnv2 mutant mice serve as a model for targeting PR to study vision (112). The mouse Kcnv2 gene (GenBank accession number: NM_183179.1; Ensembl: ENSMUSG00000047298) is located on mouse chromosome 19. Two exons have been identified, with the ATG start codon in exon 1 and TAA stop codon in exon 2. Exon 1 is selected as target sites. A gRNA targeting vector and donor oligo (with targeting sequence, flanked by 64 bp homologous sequences combined on both sides) is designed. The E314X (GAA to UAA) mutation sites in donor oligo can be introduced into exon 1 by homology-directed repair. A silent mutation can be introduced upstream so that the re-cutting of the sequence by gRNA is prevented. Cas9 mRNA, two gRNA generated by in vitro transcription, and donor oligo can be co-injected into fertilized eggs for KI mouse production. The pups are genotyped by PCR followed by sequence analysis. Positive founders can be bred to generate further homozygous colonies. A set of experiments for each strain can use AAV mediated expression, and one group of each strain can receive minicircles based expression. For each experiment, wild-type mice and mock injections serve as experimental controls.

Vision is evaluated through 1) ERG performed every two weeks up to 12 wks post-injection, 2) Fundus photo and OCT images 4 wks after injection, 3) Optomotor response at 8 wks. Imaging for marker expression is performed to establish delivery after 4-8 days. After 3 wks, some of the mice are sacrificed to image targeted delivery to PR or RPE. At the end of the experiments, eyes are harvested from control and experimental animals for retina image comparison and protein expression studies. Using our recently acquired 3 View serial block-face scanning electron microscope (SBFSEM), cellular structures are reconstructed in 3D to determine the expression of full-length protein products in the PR and RPE at its finest resolution (113, 114). The images can determine the exact expression of corrected protein products within the RPE or PR microdomains (115). Morphometric analysis is carried out for outer nuclear layer thickness and RPE cell count using frozen tissue histologic analysis. Correlation between protein and transcriptome can be established through TotalSeq™ approaches.

Compared with wild-type mice (+/+) sequence, both heterozygous (E314X/+) and homozygous (E314X/E314X) G to T mutant mice can be detected. Cas-CLOVER (Herabiolabs) or iGONAD technology can be used to generate homozygous (E314X/E314X) mice (116). These mice can survive postnatally, and all breeding will follow normal Mendelian inheritance. The mice manifest severely reduced ERG waveforms for both photopic and scotopic responses along with alteration in the outer retina structure. The truncated protein can show altered localization compared to wild-type and heterozygous mice. The phenotypic characterization can reveal cone dystrophy with supernormal rod response.

Both photopic, as well as scotopic ERG responses, and measured c-wave amplitude and implicit times, can show either normal or near-normal values (35, 117). The effect may be local, and that may have a suboptimal full-field outcome. A Celeris system can be used to measure multifocal ERG in mice to reveal any local changes. Either ex vivo ERG or whole-cell recording can be used to demonstrate the expression of channel current using pharmacological blockers. Both the Fundus photo and OCT can reveal the preservation of structure similar to wild-type or heterozygous control mice. Three-dimensional volumes of RPE cells, PR, and PR synapses can indicate protein expression and rescue of structural phenotypes (identical to wild-type retina) compared to untreated. For both Rd1 and Rd12 mice, normal PR phagocytosis (118) is expected. In the Cacna1f mice, treatment with ace-tRNA can result in crescent-shaped localization of Ca2+ channels in the 3-D reconstruction images of mouse OPL (119). A full-length protein product, in general, can correlate with no changes in cellular transcriptome profile between the treated and untreated cells. Morphology, inflammation, and physiology are expected to be consistent.

### Example 2: Initial Results of High-Fidelity Nonsense Codon Correction Generation of Minicircles for Ace-tRNA and Virus

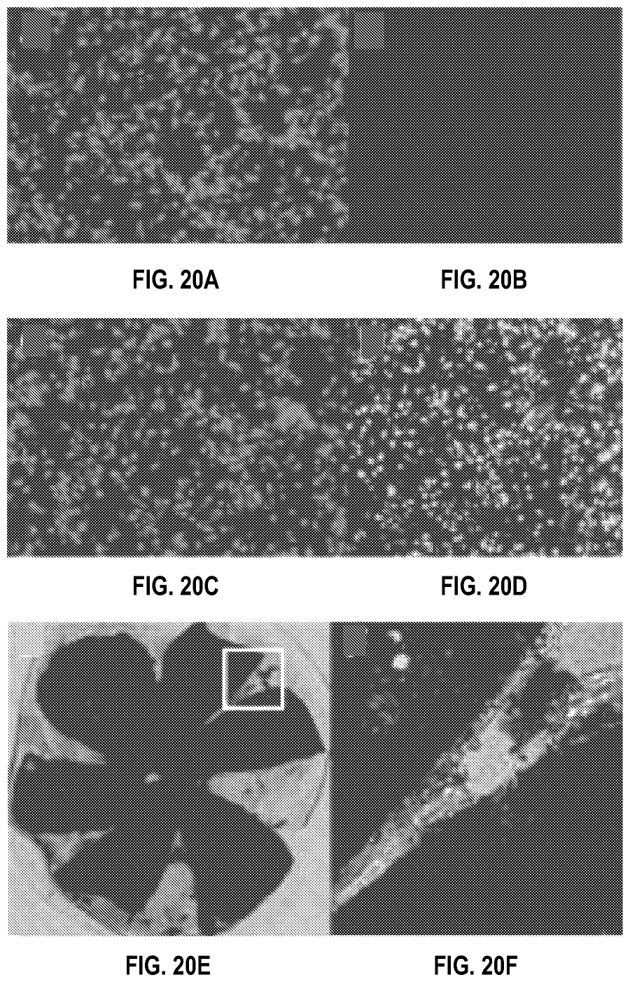

A test of 2×Arg tRNA delivery as minicircles transfected along with mOrange2 carrying a TAG mutation into HEK cells showed robust mOrange2 expression (FIG. 20A) compared to the negative control (FIG. 20B, 2×ArgRAN with tRNA sequence randomized). Lentivirus production of 3X Arg tRNA with the verification of the plasmid and also editing of both mOrange2 (FIG. 20C) and GFP (FIG. 20D) with TGA mutation. Results of GFP expression in the RPE floret (FIGS. 20E-20F) through the delivery of minicircle GFP packaged in silica nanoparticles (SNP) and injected through subretinal route are provided.

### Fabrication of Hyaluronic Acid-Coated Silica Nanoparticles (HA-SNP)

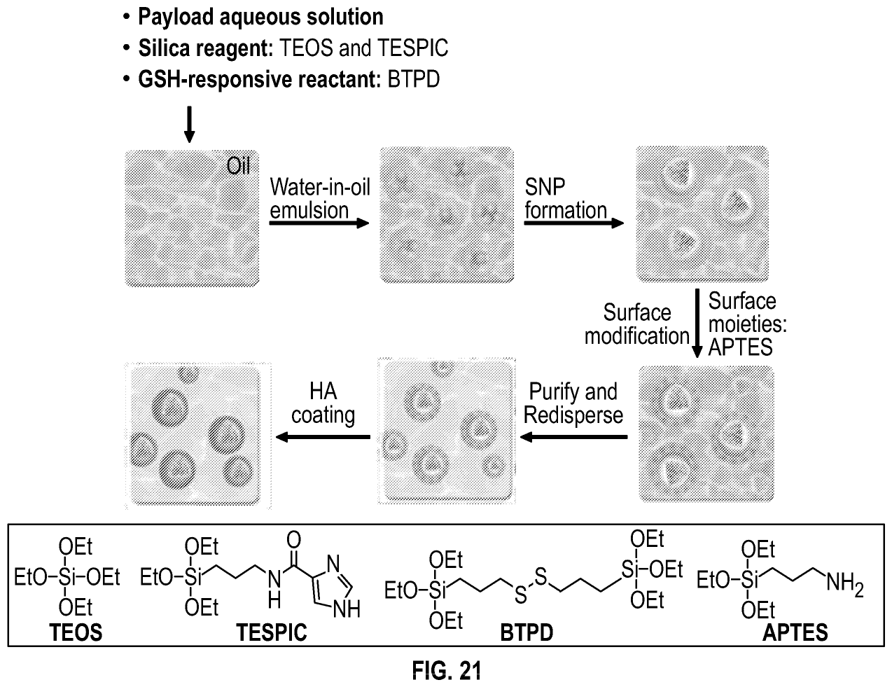

As shown in FIG. 21, silica nanoparticles (SNP) can be synthesized through a water-in-oil microemulsion method. An aqueous phase consisting of a mixture of payload and silica reagents was emulsified in an oil phase. Tetraethyl orthosilicate (TEOS) was used as a basic building block that constructs the silica network, while bis[3-(triethoxysilyl)propyl]-disulfide (BTPD) with disulfide bond was incorporated into the silica network to yield the glutathione (GSH)-responsive SNP. The surface of the SNP nanoparticles can be modified with amine groups to achieve positively charged SNP (SNP-NH2) by addition of (3-aminopropyl)triethoxysilane (APTES). SNP-NH2 can be purified by precipitation in acetone and washed by ethanol and deionized (DI) water. To obtain hyaluronic acid-coated nanoparticles, SNP-NH2 were redispersed in DI water and mixed thoroughly with an equal volume of HA aqueous solution. The size and surface charge of the HA-SNP can be adjusted by the molar ratio of HA saccharide unit/APTES. HA-SNP with a size around 50-60 nm and surface charge of −15 mV can be prepared at a ratio of HA saccharide unit/APTES=0.5 via electrostatic interactions.

### In Vivo RPE Genome Editing by SNP Via Subretinal Injection

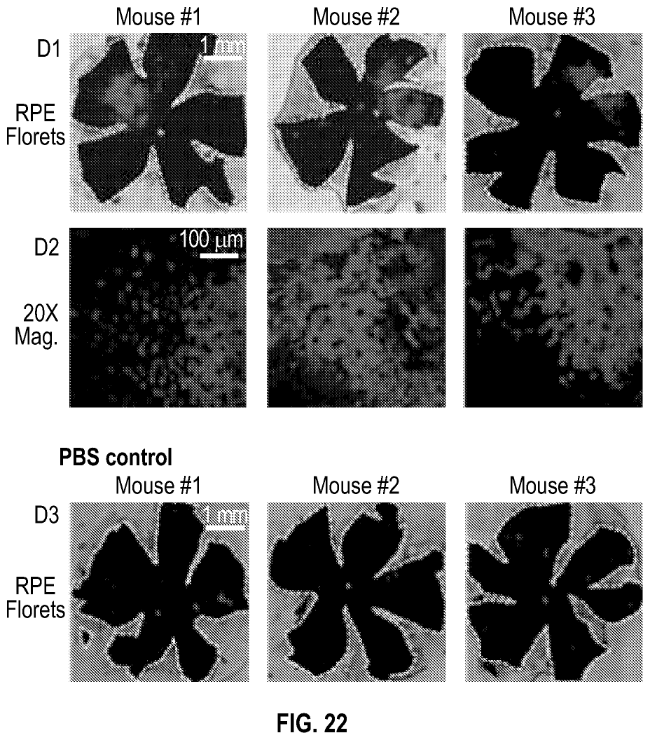

Nucleic acid delivery/genome editing efficiency of SNP (SNP-PEG) conjugated with a retinal pigment epithelium (RPE) targeting ligand all-trans-retinoic acid (ATRA) was tested in transgenic Ai14 mice (FIG. 22) (Wang et. al. PMID: 34174352; PMCID: PMC8383466). SNP-PEG-ATRA loaded with either Cre-mRNA or Cas9/sgRNA ribonucleoprotein (RNP) induced strong tdTomato expression in the RPE surrounding the subretinal injection site indicating that SNP can be a reliable nanoplatform for in vivo biomacromolecule delivery.

### In Vitro Nucleic Acid and Genome Editor Delivery by SNP

To study the DNA and RNP delivery efficiency of HA-SNP in cells, RPF (red fluorescent protein) DNA or RNP was loaded to SNP. Both SNP-NH2 and HA-SNP showed significantly higher gene transfection/editing efficiency than the Lipo 2000 control group. Moreover, there was no significant difference between SNP-NH2 and HA-SNP groups suggesting that HA coating did not impair the transfection ability of SNP.

Kir Current and Protein Expression after Ace-tRNA Treatment in HEK-FRT Cells

Much of the Kir current analysis in the HEK-FRT cells has centered around establishing high throughput patch-clamp electrophysiology. Patch solution, cell number, cell dissociation, and APC protocol have been successfully standardized, which increased the whole-cell success rate from 51% to 81%. Unlike previously reported by several groups using automated patch systems, Kir7.1 current was acquired with an inwardly rectifying current-voltage plot without the use of enhancers.

### Kv8.2—Current and Protein Expression in HEK-293 Cells

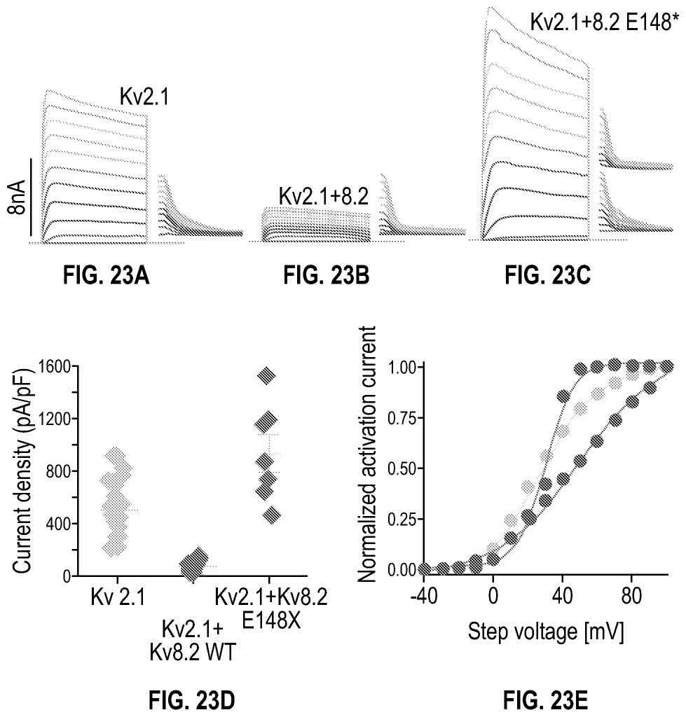

Kv8.2 is present in the photoreceptors, and as a heteromeric channel with Kv2.1, it shifts steady-state membrane potential and decreases the dark current. Several point mutations or deletions in the KCNV2 gene encode the Kv8.2 channel result in an altered rod electroretinogram, “cone dystrophy with supernormal rod response (CDSRR).” HEK293 cells with a stable expression of Kv2.1 channel (FIG. 23A) were used to overexpress Kv8.2 wildtype (FIG. 23B) or Kv8.2 E148* (FIG. 23C). The current recordings were optimized using a high throughput automated patch-clamp device QPatchII. Expression of Kv8.2 reduced current amplitude (FIGS. 23B, 23D) and shifted the activation curve (FIG. 23E). Co-expression of Kv8.2 E148*, which likely results in a truncated protein product, did not affect the current or activation curve of the Kv2.1 channel, confirming the dominant dominant-negative effect Kv8.2 on Kv2.1 current to reproduce disease phenotype.

Establishing and Characterizing BEST1R200 iPSC-RPE

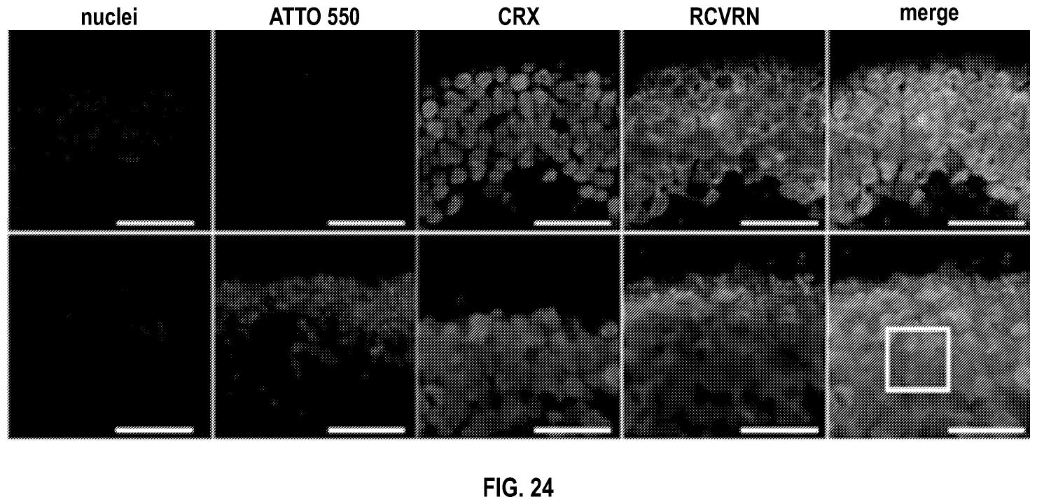

A payload delivery to the photoreceptors (PRs) in stage 3 3D retinal organoids was tested (FIG. 24). In this example, a 3D organoid was treated with 15 ug/well SNP conjugated to cell-penetrating peptide (CPP) carrying a payload of nuclear-localized Cas9/gRNA complexed RNP 10% conjugated to ATTO 550. Half media was replaced after 24 hrs due to osmotic toxicity. After removing excess SNP, the organoids were fixed and imaged. The upper panel in the figure represents untransduced organoids, and the lower panels are images from transduced organoids. In the figure, ATTO 550 appeared to be localized in the interphotoreceptor matrix and the nuclei of both CRX+ and CRX− cells.

### Generation of Kcnv2 and Cacna1f Mutant Mice

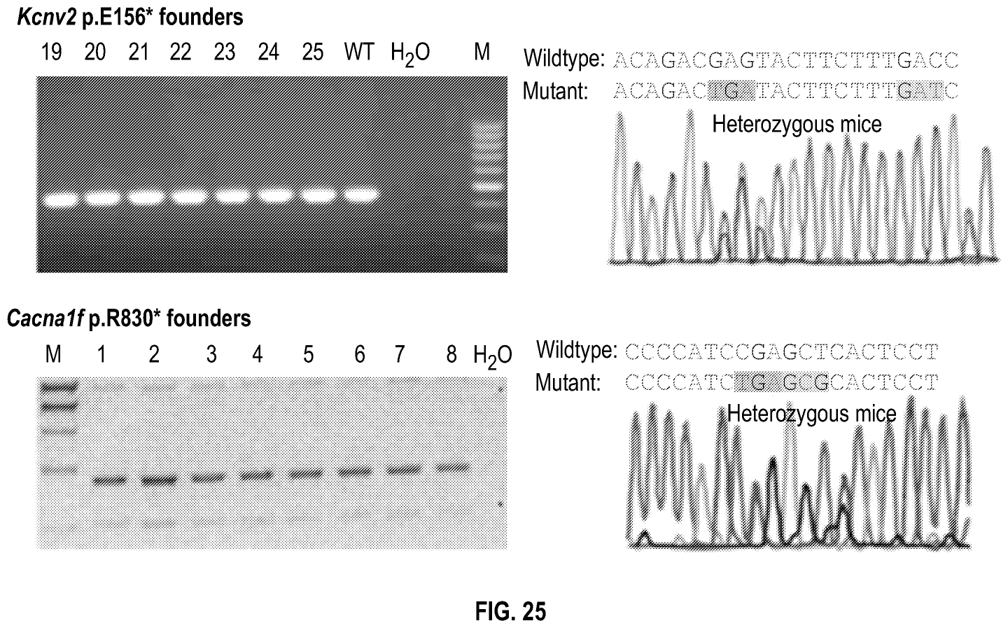

Nonsense mutations in human KCNV2 and CACNA1f cause CDSSR and congenital stationary night blindness (CSNB), respectively. Both to study the mechanisms that link genetic mutations to retinal dystrophy and test the effectiveness of ace-tRNA to treat channelopathies, mouse models have been generated. These models were developed by genome editing using Cas9, respective guide RNA, and 64 nucleotide donor DNA containing required mutations. Zygotes were microinjected to surrogate mothers, and pups were genotyped. As in FIG. 25, sequencing the PCR product identified heterozygous mice Kcnv2 pE156* (GAG to TGA) with silent mutation (GAC to GAT) and Cacna1f pR830* (CGA to TGA) with silent mutation (GCT to GCG). PCR product confirmed the gene mutations in founders.

### REFERENCES

1. Atkinson J, Martin R. Mutations to nonsense codons in human genetic disease: implications for gene therapy by nonsense suppressor tRNAs. Nucleic Acids Res. 1994; 22(8):1327-34. Epub 1994 Apr. 25. PubMed PMID: 8190621; PMCID: PMC307985.
2. Linde L, Boelz S, Nissim-Rafinia M, Oren Y S, Wilschanski M, Yaacov Y, Virgilis D, Neu-Yilik G, Kulozik A E, Kerem E, Kerem B. Nonsense-mediated mRNA decay affects nonsense transcript levels and governs response of cystic fibrosis patients to gentamicin. J Clin Invest. 2007; 117(3):683-92. Epub 2007 Feb. 10. doi: 10.1172/JCI28523. PubMed PMID: 17290305; PMCID: PMC1783999.
3. Liu H X, Cartegni L, Zhang M Q, Krainer A R. A mechanism for exon skipping caused by nonsense or missense mutations in BRCA1 and other genes. Nat Genet. 2001; 27(1):55-8. Epub 2001 Jan. 4. doi: 10.1038/83762. PubMed PMID: 11137998.
4. Mort M, Ivanov D, Cooper D N, Chuzhanova N A. A meta-analysis of nonsense mutations causing human genetic disease. Hum Mutat. 2008; 29(8):1037-47. Epub 2008 May 6. doi: 10.1002/humu.20763. PubMed PMID: 18454449.
5. Howard M, Frizzell R A, Bedwell D M. Aminoglycoside antibiotics restore CFTR function by overcoming premature stop mutations. Nat Med. 1996; 2(4):467-9. PubMed PMID: 8597960.
6. Arakawa M, Shiozuka M, Nakayama Y, Hara T, Hamada M, Kondo S, Ikeda D, Takahashi Y, Sawa R, Nonomura Y, Sheykholeslami K, Kondo K, Kaga K, Kitamura T, Suzuki-Miyagoe Y, Takeda S, Matsuda R. Negamycin restores dystrophin expression in skeletal and cardiac muscles of mdx mice. J Biochem. 2003; 134(5): 751-8. PubMed PMID: 14688241.
7. Welch E M, Barton E R, Zhuo J, Tomizawa Y, Friesen W J, Trifillis P, Paushkin S, Patel M, Trotta C R, Hwang S, Wilde R G, Karp G, Takasugi J, Chen G, Jones S, Ren H, Moon Y C, Corson D, Turpoff A A, Campbell J A, Conn M M, Khan A, Almstead N G, Hedrick J, Mollin A, Risher N, Weetall M, Yeh S, Branstrom A A, Colacino J M, Babiak J, Ju W D, Hirawat S, Northcutt V J, Miller L L, Spatrick P, He F, Kawana M, Feng H, Jacobson A, Peltz S W, Sweeney H L. PTC124 targets genetic disorders caused by nonsense mutations. Nature. 2007; 447(7140):87-91. doi: 10.1038/nature05756. PubMed PMID: 17450125.
8. Shahi P K, Hermans D, Sinha D, Brar S, Moulton H, Stulo S, Borys K D, Capowski E, Pillers D M, Gamm D M, Pattnaik B R. Gene Augmentation and Readthrough Rescue Channelopathy in an iPSC-RPE Model of Congenital Blindness. Am J Hum Genet. 2019; 104(2):310-8. Epub 2019 Jan. 29. doi: 10.1016/j.ajhg.2018.12.019. PubMed PMID: 30686507; PMCID: PMC6369573.
9. Roy B, Friesen W J, Tomizawa Y, Leszyk J D, Zhuo J, Johnson B, Dakka J, Trotta C R, Xue X, Mutyam V, Keeling K M, Mobley J A, Rowe S M, Bedwell D M, Welch E M, Jacobson A. Ataluren stimulates ribosomal selection of near-cognate tRNAs to promote nonsense suppression. Proc Natl Acad Sci USA. 2016; 113(44):12508-13. doi: 10.1073/pnas. 1605336113. PubMed PMID: 27702906; PMCID: PMC5098639.
10. Berecki G, Bryson A, Terhag J, Maljevic S, Gazina E V, Hill S L, Petrou S. SCN1A gain of function in early infantile encephalopathy. Ann Neurol. 2019; 85(4):514-25. Epub 2019 Feb. 20. doi: 10.1002/ana.25438. PubMed PMID: 30779207.
11. Dhifallah S, Lancaster E, Merrill S, Leroudier N, Mantegazza M, Cestele S. Gain of Function for the SCN1A/hNav1.1-L1670W Mutation Responsible for Familial Hemiplegic Migraine. Front Mol Neurosci. 2018; 11:232. Epub 2018 Jul. 25. doi: 10.3389/fnmol.2018.00232. PubMed PMID: 30038559; PMCID: PMC6046441.
12. Kotecha B, Richardson GP. Ototoxicity in vitro: effects of neomycin, gentamicin, dihydrostreptomycin, amikacin, spectinomycin, neamine, spermine and poly-L-lysine. Hear Res. 1994; 73(2):173-84. PubMed PMID: 7514588.
13. Ledford H. CRISPR treatment inserted directly into the body for first time. Nature. 2020; 579(7798):185. Epub 2020 Mar. 12. doi: 10.1038/d41586-020-00655-8. PubMed PMID: 32157225.
14. Dai W J, Zhu L Y, Yan Z Y, Xu Y, Wang Q L, Lu X J. CRISPR-Cas9 for in vivo Gene Therapy: Promise and Hurdles. Mol Ther Nucleic Acids. 2016; 5:e349. Epub 2017 Jan. 31. doi: 10.1038/mtna.2016.58. PubMed PMID: 28131272; PMCID: PMC5023403.
15. Peng R, Lin G, Li J. Potential pitfalls of CRISPR/Cas9-mediated genome editing. FEBS J. 2016; 283(7):1218-31. Epub 2015 Nov. 5. doi: 10.1111/febs.13586. PubMed PMID: 26535798.
16. Lueck J D, Yoon J S, Perales-Puchalt A, Mackey A L, Infield D T, Behlke M A, Pope M R, Weiner D B, Skach W R, McCray P B, Jr., Ahern C A. Engineered transfer RNAs for suppression of premature termination codons. Nat Commun. 2019; 10(1):822. Epub 2019 Feb. 20. doi: 10.1038/s41467-019-08329-4. PubMed PMID: 30778053; PMCID: PMC6379413.
17. Gonzalez-Duarte R, de Castro-Miro M, Tuson M, Ramirez-Castaneda V, Gils R V, Marfany G. Scaling New Heights in the Genetic Diagnosis of Inherited Retinal Dystrophies. Adv Exp Med Biol. 2019; 1185:215-9. Epub 2019 Dec. 31. doi: 10.1007/978-3-030-27378-1 35. PubMed PMID: 31884614.
18. Garafalo A V, Cideciyan A V, Heon E, Sheplock R, Pearson A, WeiYang Yu C, Sumaroka A, Aguirre G D, Jacobson S G. Progress in treating inherited retinal diseases: Early subretinal gene therapy clinical trials and candidates for future initiatives. Prog Retin Eye Res. 2019:100827. Epub 2020 Jan. 4. doi: 10.1016/j.preteyeres.2019.100827. PubMed PMID: 31899291.
19. Lakkaraju A, Umapathy A, Tan L X, Daniele L, Philp N J, Boesze-Battaglia K, Williams D S. The cell biology of the retinal pigment epithelium. Prog Retin Eye Res. 2020:100846. Epub 2020 Feb. 28. doi: 10.1016/j.preteyeres.2020.100846. PubMed PMID: 32105772.
20. Schatz P, Klar J, Andreasson S, Ponjavic V, Dahl N. Variant phenotype of Best vitelliform macular dystrophy associated with compound heterozygous mutations in VMD2. Ophthalmic Genet. 2006; 27(2):51-6. Epub 2006 Jun. 7. doi: 10.1080/13816810600677990. PubMed PMID: 16754206.
21. Burgess R, Millar I D, Leroy B P, Urquhart J E, Fearon I M, De Baere E, Brown P D, Robson A G, Wright G A, Kestelyn P, Holder G E, Webster A R, Manson F D, Black G C. Biallelic mutation of BEST1 causes a distinct retinopathy in humans. Am J Hum Genet. 2008; 82(1):19-31. Epub 2008 Jan. 9. doi: 10.1016/j.ajhg.2007.08.004. PubMed PMID: 18179881; PMCID: PMC2253971.
22. Chibani Z, Abid I Z, Molbaek A, Soderkvist P, Feki J, Hmani-Aifa M. Novel BEST1 gene mutations associated with two different forms of macular dystrophy in Tunisian families. Clin Exp Ophthalmol. 2019; 47(8):1063-73. Epub 2019 Jun. 30. doi: 10.1111/ceo.13577. PubMed PMID: 31254423.
23. Wu H, Cowing J A, Michaelides M, Wilkie S E, Jeffery G, Jenkins S A, Mester V, Bird A C, Robson A G, Holder G E, Moore A T, Hunt D M, Webster A R. Mutations in the gene KCNV2 encoding a voltage-gated potassium channel subunit cause “cone dystrophy with supernormal rod electroretinogram” in humans. Am J Hum Genet. 2006; 79(3): 574-9. Epub 2006 Aug. 16. doi: 10.1086/507568. PubMed PMID: 16909397; PMCID: PMC1559534.
24. Wissinger B, Dangel S, Jagle H, Hansen L, Baumann B, Rudolph G, Wolf C, Bonin M, Koeppen K, Ladewig T, Kohl S, Zrenner E, Rosenberg T. Cone dystrophy with supernormal rod response is strictly associated with mutations in KCNV2. Invest Ophthalmol Vis Sci. 2008; 49(2): 751-7. Epub 2008 Feb. 1. doi: 10.1167/iovs.07-0471. PubMed PMID: 18235024.
25. Wycisk K A, Budde B, Feil S, Skosyrski S, Buzzi F, Neidhardt J, Glaus E, Nurnberg P, Ruether K, Berger W. Structural and functional abnormalities of retinal ribbon synapses due to Cacna2d4 mutation. Invest Ophthalmol Vis Sci. 2006; 47(8): 3523-30. Epub 2006 Aug. 1. doi: 10.1167/iovs.06-0271. PubMed PMID: 16877424.
26 Ba-Abbad R, Arno G, Carss K, Stirrups K, Penkett C J, Moore A T, Michaelides M, Raymond F L, Webster A R, Holder G E. Mutations in CACNA2D4 Cause Distinctive Retinal Dysfunction in Humans. Ophthalmology. 2016; 123(3):668-71 e2. Epub 2015 Nov. 13. doi: 10.1016/j.ophtha.2015.09.045. PubMed PMID: 26560832.
27. Strom T M, Nyakatura G, Apfelstedt-Sylla E, Hellebrand H, Lorenz B, Weber B H, Wutz K, Gutwillinger N, Ruther K, Drescher B, Sauer C, Zrenner E, Meitinger T, Rosenthal A, Meindl A. An L-type calcium-channel gene mutated in incomplete X-linked congenital stationary night blindness. Nat Genet. 1998; 19(3):260-3. Epub 1998 Jul. 14. doi: 10.1038/940. PubMed PMID: 9662399.
28. Bech-Hansen N T, Naylor M J, Maybaum T A, Pearce W G, Koop B, Fishman G A, Mets M, Musarella M A, Boycott K M. Loss-of-function mutations in a calcium-channel alpha1-subunit gene in Xp11.23 cause incomplete X-linked congenital stationary night blindness. Nat Genet. 1998; 19(3):264-7. Epub 1998 Jul. 14. doi: 10.1038/947. PubMed PMID: 9662400.
29. Pattnaik B R, Shahi P K, Marino M J, Liu X, York N, Brar S, Chiang J, Pillers D A, Traboulsi E I. A Novel KCNJ13 Nonsense Mutation and Loss of Kir7.1 Channel Function Causes Leber Congenital Amaurosis (LCA16). Hum Mutat. 2015; 36(7):720-7. Epub 2015 Apr. 30. doi: 10.1002/humu.22807. PubMed PMID: 25921210.
30. Sergouniotis P I, Davidson A E, Mackay D S, Li Z, Yang X, Plagnol V, Moore A T, Webster A R. Recessive mutations in KCNJ13, encoding an inwardly rectifying potassium channel subunit, cause leber congenital amaurosis. Am J Hum Genet. 2011; 89(1):183-90. Epub 2011 Jul. 19. doi: 10.1016/j.ajhg.2011.06.002. PubMed PMID: 21763485; PMCID: PMC3135807.
31. Audo I, Kohl S, Leroy B P, Munier F L, Guillonneau X, Mohand-Said S, Bujakowska K, Nandrot E F, Lorenz B, Preising M, Kellner U, Renner A B, Bernd A, Antonio A, Moskova-Doumanova V, Lancelot M E, Poloschek C M, Drumare I, Defoort-Dhellemmes S, Wissinger B, Leveillard T, Hamel C P, Schorderet D F, De Baere E, Berger W, Jacobson S G, Zrenner E, Sahel J A, Bhattacharya S S, Zeitz C. TRPM1 is mutated in patients with autosomal-recessive complete congenital stationary night blindness. Am J Hum Genet. 2009; 85(5):720-9. Epub 2009 Nov. 10. doi: 10.1016/j.ajhg.2009.10.013. PubMed PMID: 19896113; PMCID: PMC2775830.
32. AlTalbishi A, Zelinger L, Zeitz C, Hendler K, Namburi P, Audo I, Sheffer R, Yahalom C, Khateb S, Banin E, Sharon D. TRPM1 Mutations are the Most Common Cause of Autosomal Recessive Congenital Stationary Night Blindness (CSNB) in the Palestinian and Israeli Populations. Sci Rep. 2019; 9(1):12047. Epub 2019 Aug. 21. doi: 10.1038/s41598-019-46811-7. PubMed PMID: 31427709; PMCID: PMC6700182.
33. Nakamura M, Sanuki R, Yasuma T R, Onishi A, Nishiguchi K M, Koike C, Kadowaki M, Kondo M, Miyake Y, Furukawa T. TRPM1 mutations are associated with the complete form of congenital stationary night blindness. Mol Vis. 2010; 16:425-37. Epub 2010 Mar. 20. PubMed PMID: 20300565; PMCID: PMC2838739.
34. Yang D, Pan A, Swaminathan A, Kumar G, Hughes BA. Expression and localization of the inwardly rectifying potassium channel Kir7.1 in native bovine retinal pigment epithelium. Invest Ophthalmol Vis Sci. 2003; 44(7):3178-85. Epub 2003 Jun. 26. PubMed PMID: 12824269.
35. Shahi P K, Liu X, Aul B, Moyer A, Pattnaik A, Denton J, Pillers D M, Pattnaik B R. Abnormal Electroretinogram after Kir7.1 Channel Suppression Suggests Role in Retinal Electrophysiology. Sci Rep. 2017; 7(1):10651. Epub 2017 Sep. 8. doi: 10.1038/s41598-017-11034-1. PubMed PMID: 28878288; PMCID: PMC5587531.
36. Khan A O, Bergmann C, Neuhaus C, Bolz H J. A distinct vitreo-retinal dystrophy with early-onset cataract from recessive KCNJ13 mutations. Ophthalmic Genet. 2015; 36(1):79-84. Epub 2014 Dec. 6. doi: 10.3109/13816810.2014.985846. PubMed PMID: 25475713.
37. Perez-Roustit S, Marquette V, Bocquet B, Kaplan J, Perrault I, Meunier I, Hamel C P. Leber Congenital Amaurosis with Large Retinal Pigment Clumps Caused by Compound Heterozygous Mutations in Konj13. Retin Cases Brief Rep. 2017; 11(3):221-6. Epub 2016 May 21. doi: 10.1097/ICB.0000000000000326. PubMed PMID: 27203561.
38. Toms M, Dubis A M, Lim W S, Webster A R, Gorin M B, Moosajee M. Missense variants in the conserved transmembrane M2 protein domain of KCNJ13 associated with retinovascular changes in humans and zebrafish. Exp Eye Res. 2019; 189:107852. Epub 2019 Oct. 28. doi: 10.1016/j.exer.2019.107852. PubMed PMID: 31647904; PMCID: PMC6899441.
39. Ottschytsch N, Raes A, Van Hoorick D, Snyders D J. Obligatory heterotetramerization of three previously uncharacterized Kv channel alpha-subunits identified in the human genome. Proc Natl Acad Sci USA. 2002; 99(12):7986-91. Epub 2002 Jun. 13. doi: 10.1073/pnas. 122617999. PubMed PMID: 12060745; PMCID: PMC123007.
40. Zobor D, Kohl S, Wissinger B, Zrenner E, Jagle H. Rod and cone function in patients with KCNV2 retinopathy. PLOS One. 2012; 7(10):e46762. Epub 2012 Oct. 19. doi: 10.1371/journal.pone.0046762. PubMed PMID: 23077521; PMCID: PMC3471896.
41. Marmorstein A D, Marmorstein L Y, Rayborn M, Wang X, Hollyfield J G, Petrukhin K. Bestrophin, the product of the Best vitelliform macular dystrophy gene (VMD2), localizes to the basolateral plasma membrane of the retinal pigment epithelium. Proc Natl Acad Sci USA. 2000; 97(23):12758-63. Epub 2000 Oct. 26. doi: 10.1073/pnas.220402097 220402097 [pii]. PubMed PMID: 11050159; PMCID: 18837.
42. Tsunenari T, Sun H, Williams J, Cahill H, Smallwood P, Yau K W, Nathans J. Structure-function analysis of the bestrophin family of anion channels. J Biol Chem. 2003; 278(42):41114-25. Epub 2003 Aug. 9. doi: 10.1074/jbc.M306150200 M306150200 [pii]. PubMed PMID: 12907679; PMCID: 2885917.
43. Qin N, Yagel S, Momplaisir M L, Codd E E, D'Andrea M R. Molecular cloning and characterization of the human voltage-gated calcium channel alpha(2)delta-4 subunit. Mol Pharmacol. 2002; 62(3):485-96. Epub 2002 Aug. 16. doi: 10.1124/mol.62.3.485. PubMed PMID: 12181424.
44. Baumann L, Gerstner A, Zong X, Biel M, Wahl-Schott C. Functional characterization of the L-type Ca2+ channel Cav1.4alpha1 from mouse retina. Invest Ophthalmol Vis Sci. 2004; 45(2):708-13. Epub 2004 Jan. 28. doi: 10.1167/iovs.03-0937. PubMed PMID: 14744918.
45. Morgans C W. Localization of the alpha(1F) calcium channel subunit in the rat retina. Invest Ophthalmol Vis Sci. 2001; 42(10): 2414-8. Epub 2001 Aug. 31. PubMed PMID: 11527958.
46. Bech-Hansen N T, Boycott K M, Gratton K J, Ross D A, Field L L, Pearce W G. Localization of a gene for incomplete X-linked congenital stationary night blindness to the interval between DXS6849 and DXS8023 in Xp11.23. Hum Genet. 1998; 103(2):124-30. Epub 1998 Oct. 6. doi: 10.1007/s004390050794. PubMed PMID: 9760193.
47. Prado D A, Acosta-Acero M, Maldonado R S. Gene therapy beyond luxturna: a new horizon of the treatment for inherited retinal disease. Curr Opin Ophthalmol. 2020. Epub 2020 Mar. 17. doi: 10.1097/ICU.0000000000000660. PubMed PMID: 32175942.
48. Tokhmafshan F, Dickinson K, Akpa M M, Brasell E, Huertas P, Goodyer P R. A no-nonsense approach to hereditary kidney disease. Pediatr Nephrol. 2019. Epub 2019 Dec. 7. doi: 10.1007/s00467-019-04394-5. PubMed PMID: 31807928.
49. Cohen S Y, Mimoun G, Oubraham H, Zourdani A, Malbrel C, Quere S, Schneider V, Group L S. Changes in visual acuity in patients with wet age-related macular degeneration treated with intravitreal ranibizumab in daily clinical practice: the LUMIERE study. Retina. 2013; 33(3):474-81. Epub 2012 Dec. 26. doi: 10.1097/IAE.0b013e31827b6324. PubMed PMID: 23266880.
50. Weng CY. Bilateral Subretinal Voretigene Neparvovec-rzyl (Luxturna) Gene Therapy. Ophthalmol Retina. 2019; 3(5):450. Epub 2019 May 3. doi: 10.1016/j.oret.2019.02.007. PubMed PMID: 31044739.
51. Luaces-Rodriguez A, Mondelo-Garcia C, Zarra-Ferro I, Gonzalez-Barcia M, Aguiar P, Fernandez-Ferreiro A, Otero-Espinar F J. Intravitreal anti-VEGF drug delivery systems for age-related macular degeneration. Int J Pharm. 2020; 573:118767. Epub 2019 Nov. 2. doi: 10.1016/j.ijpharm.2019.118767. PubMed PMID: 31669558.
52. Edelhauser H F, Boatright J H, Nickerson J M, Third APRIWG. Drug delivery to posterior intraocular tissues: third Annual ARVO/Pfizer Ophthalmics Research Institute Conference. Invest Ophthalmol Vis Sci. 2008; 49(11):4712-20. Epub 2008 Aug. 19. doi: 10.1167/iovs.08-1904. PubMed PMID: 18708617; PMCID: PMC2633627.
53. Jiang S, Franco Y L, Zhou Y, Chen J. Nanotechnology in retinal drug delivery. Int J Ophthalmol. 2018; 11(6):1038-44. Epub 2018 Jul. 7. doi: 10.18240/ijo.2018.06.23. PubMed PMID: 29977820; PMCID: PMC6010371.
54. Huang X, Chau Y. Intravitreal nanoparticles for retinal delivery. Drug discovery today. 2019.
55. Koo H, Moon H, Han H, Na J H, Huh M S, Park J H, Woo S J, Park K H, Kwon I C, Kim K. The movement of self-assembled amphiphilic polymeric nanoparticles in the vitreous and retina after intravitreal injection. Biomaterials. 2012; 33(12):3485-93.
56. Lowe T M, Chan P P. tRNAscan-SE On-line: integrating search and context for analysis of transfer RNA genes. Nucleic Acids Res. 2016; 44(W1):W54-7. doi: 10.1093/nar/gkw413. PubMed PMID: 27174935; PMCID: PMC4987944.
57. Lowe T M, Eddy S R. tRNAscan-SE: a program for improved detection of transfer RNA genes in genomic sequence. Nucleic Acids Res. 1997; 25(5):955-64. PubMed PMID: 9023104; PMCID: PMC146525.
58. Sun D, Sahu B, Gao S, Schur R M, Vaidya A M, Maeda A, Palczewski K, Lu Z-R. Targeted multifunctional lipid ECO plasmid DNA nanoparticles as efficient non-viral gene therapy for leber's congenital amaurosis. Molecular Therapy-Nucleic Acids. 2017; 7:42-52.
59. Carlson A, Bok D. Promotion of the release of 11-cis-retinal from cultured retinal pigment epithelium by interphotoreceptor retinoid-binding protein. Biochemistry-us. 1992; 31(37):9056-62.
60. Madisen L, Zwingman T A, Sunkin S M, Oh S W, Zariwala H A, Gu H, Ng L L, Palmiter R D, Hawrylycz M J, Jones A R. A robust and high-throughput Cre reporting and characterization system for the whole mouse brain. Nature neuroscience. 2010; 13(1):133.
61. Staahl B T, Benekareddy M, Coulon-Bainier C, Banfal A A, Floor S N, Sabo J K, Urnes C, Munares G A, Ghosh A, Doudna J A. Efficient genome editing in the mouse brain by local delivery of engineered Cas9 ribonucleoprotein complexes. Nature Biotechnology. 2017; 35(5):431-4.
62. Smith E N, D'Antonio-Chronowska A, Greenwald W W, Borja V, Aguiar L R, Pogue R, Matsui H, Benaglio P, Borooah S, D'Antonio M. Human iPSC-derived retinal pigment epithelium: a model system for prioritizing and functionally characterizing causal variants at AMD risk loci. Stem cell reports. 2019; 12(6):1342-53.
63. Maruotti J, Sripathi S R, Bharti K, Fuller J, Wahlin K J, Ranganathan V, Sluch V M, Berlinicke C A, Davis J, Kim C. Small-molecule-directed, efficient generation of retinal pigment epithelium from human pluripotent stem cells. Proc Natl Acad Sci USA. 2015; 112(35):10950-5.
64. Williams L M CaRW, editor. Eye, Retin, and Visual System of the Mouse. 1 ed. Cambridge Massachusetts, and London England: The MIT Press; 2008.
65. Zhong H, Chen Y, Li Y, Chen R, Mardon G. CRISPR-engineered mosaicism rapidly reveals that loss of Kcnj13 function in mice mimics human disease phenotypes. Sci Rep. 2015; 5:8366. doi: 10.1038/srep08366. PubMed PMID: 25666713; PMCID: 4322368.
66. Phillips M J, Wallace K A, Dickerson S J, Miller M J, Verhoeven A D, Martin J M, Wright L S, Shen W, Capowski E E, Percin E F, Perez E T, Zhong X, Canto-Soler M V, Gamm D M. Blood-derived human iPS cells generate optic vesicle-like structures with the capacity to form retinal laminae and develop synapses. Invest Ophthalmol Vis Sci. 2012; 53(4):2007-19. doi: 10.1167/iovs.11-9313. PubMed PMID: 22410558; PMCID: 3648343.
67. Meyer J S, Howden S E, Wallace K A, Verhoeven A D, Wright L S, Capowski E E, Pinilla I, Martin J M, Tian S, Stewart R, Pattnaik B, Thomson J A, Gamm D M. Optic vesicle-like structures derived from human pluripotent stem cells facilitate a customized approach to retinal disease treatment. Stem Cells. 2011; 29(8):1206-18. Epub 2011 Jun. 17. doi: 10.1002/stem.674. PubMed PMID: 21678528.
68. Meyer J S, Shearer R L, Capowski E E, Wright L S, Wallace K A, McMillan E L, Zhang S C, Gamm D M. Modeling early retinal development with human embryonic and induced pluripotent stem cells. Proc Natl Acad Sci USA. 2009; 106(39):16698-703. Epub 2009 Aug. 27. doi: 10.1073/pnas.0905245106. PubMed PMID: 19706890; PMCID: 2757802.
69. Dabrowski M, Bukowy-Bieryllo Z, Zietkiewicz E. Advances in therapeutic use of a drug-stimulated translational readthrough of premature termination codons. Mol Med. 2018; 24(1):25. Epub 2018 Aug. 24. doi: 10.1186/s10020-018-0024-7. PubMed PMID: 30134808; PMCID: PMC6016875.
70. Liu X, Fu Y, Yang H, Mavlyutov T, Li J, McCurdy C R, Guo L W, Pattnaik B R. Potential independent action of sigma receptor ligands through inhibition of the Kv2.1 channel. Oncotarget. 2017; 8(35):59345-58. Epub 2017 Sep. 25. doi: 10.18632/oncotarget.19581. PubMed PMID: 28938641; PMCID: PMC5601737.
71. Pattnaik B R, Tokarz S, Asuma M P, Schroeder T, Sharma A, Mitchell J C, Edwards A O, Pillers D A. Snowflake vitreoretinal degeneration (SVD) mutation R162W provides new insights into Kir7.1 ion channel structure and function. PLOS One. 2013; 8(8):e71744. Epub 2013 Aug. 27. doi: 10.1371/journal.pone.0071744. PubMed PMID: 23977131; PMCID: PMC3747230.
72. Pattnaik B R, Hughes B A. Effects of KCNQ channel modulators on the M-type potassium current in primate retinal pigment epithelium. Am J Physiol Cell Physiol. 2012; 302(5):C821-33. Epub 2011 Dec. 3. doi: 10.1152/ajpcell.00269.2011. PubMed PMID: 22135213; PMCID: PMC3311298.
73. Peloquin J B, Rehak R, Doering C J, McRory J E. Functional analysis of congenital stationary night blindness type-2 CACNAIF mutations F742C, G1007R, and R1049W. Neuroscience. 2007; 150(2):335-45. Epub 2007 Oct. 24. doi: 10.1016/j.neuroscience.2007.09.021. PubMed PMID: 17949918.
74. Sinha D, Steyer B, Shahi P K, Mueller K, Valiauga R, Edwards K L, Bacig C, Steltzer S S, Srinivasan S, Abdeen A, Cory E, Periyasamy V, Siahpirani A F, Stone E M, Tucker B A, Roy S, Pattnaik B R, Saha K, Gamm D M. Human iPSC modeling reveals mutation-specific responses to gene therapy in Best disease. bioRxiv. 2020:796581. doi: 10.1101/796581.
75. Gaspar V, de Melo-Diogo D, Costa E, Moreira A, Queiroz J, Pichon C, Correia I, Sousa F. Minicircle DNA vectors for gene therapy: advances and applications. Expert Opin Biol Ther. 2015; 15(3):353-79. Epub 2014 Dec. 30. doi: 10.1517/14712598.2015.996544. PubMed PMID: 25539147.
76. Darquet A M, Cameron B, Wils P, Scherman D, Crouzet J. A new DNA vehicle for nonviral gene delivery: supercoiled minicircle. Gene Ther. 1997; 4(12):1341-9. Epub 1998 Feb. 24. doi: 10.1038/sj.gt.3300540. PubMed PMID: 9472558.
77. Chen Z Y, He C Y, Meuse L, Kay M A. Silencing of episomal transgene expression by plasmid bacterial DNA elements in vivo. Gene Ther. 2004; 11(10):856-64. Epub 2004 Mar. 19. doi: 10.1038/sj.gt.3302231. PubMed PMID: 15029228.
78. Mayrhofer P, Schleef M, Jechlinger W. Use of minicircle plasmids for gene therapy. Methods Mol Biol. 2009; 542:87-104. Epub 2009 Jul. 2. doi: 10.1007/978-1-59745-561-9 4. PubMed PMID: 19565897.
79. Hibbitt O C, McNeil E, Lufino M M, Seymour L, Channon K, Wade-Martins R. Long-term physiologically regulated expression of the low-density lipoprotein receptor in vivo using genomic DNA mini-gene constructs. Mol Ther. 2010; 18(2):317-26. Epub 2009 Oct. 29. doi: 10.1038/mt.2009.249. PubMed PMID: 19861949; PMCID: PMC2839284.
80. Barnea-Cramer A O, Singh M, Fischer D, De Silva S, McClements M E, Barnard A R, MacLaren R E. Repair of Retinal Degeneration following Ex Vivo Minicircle DNA Gene Therapy and Transplantation of Corrected Photoreceptor Progenitors. Mol Ther. 2020; 28(3):830-44. Epub 2020 Feb. 7. doi: 10.1016/j.ymthe.2020.01.023. PubMed PMID: 32027843; PMCID: PMC7054814.
81. Conley S M, Cai X, Naash M I. Nonviral ocular gene therapy: assessment and future directions. Curr Opin Mol Ther. 2008; 10(5):456-63. Epub 2008 Oct. 3. PubMed PMID: 18830921; PMCID: PMC2938038.
82. Liu N, Wang B J, Broughton K M, Alvarez R, Siddiqi S, Loaiza R, Nguyen N, Quijada P, Gude N, Sussman M A. PIM1-minicircle as a therapeutic treatment for myocardial infarction. PLOS One. 2017; 12(3):e0173963. Epub 2017 Mar. 23. doi: 10.1371/journal.pone.0173963. PubMed PMID: 28323876; PMCID: PMC5360264.
83. Xiong W, Wu D M, Xue Y, Wang S K, Chung M J, Ji X, Rana P, Zhao S R, Mai S, Cepko C L. AAV cis-regulatory sequences are correlated with ocular toxicity. Proc Natl Acad Sci USA. 2019; 116(12):5785-94. Epub 2019 Mar. 6. doi: 10.1073/pnas.1821000116. PubMed PMID: 30833387; PMCID: PMC6431174.
84. J Cehajic-Kapetanovic A A, R J Lucas, P N Bishop Outer retinal transduction can be achieved after intravitreal delivery of adeno-associated virus serotype 2 vector with glycosidic enzymes. The Lancet. 2013; 381(Supplement 1). doi: 10.1016/S0140-6736(13)60469-9.
85. Kim H, Robinson S B, Csaky K G. Investigating the movement of intravitreal human serum albumin nanoparticles in the vitreous and retina. Pharmaceutical research. 2009; 26(2):329-37.
86. Schön C, Biel M, Michalakis S. Retinal gene delivery by adeno-associated virus (AAV) vectors: Strategies and applications. European Journal of Pharmaceutics and Biopharmaceutics. 2015; 95:343-52. doi: https://doi.org/10.1016/j.ejpb.2015.01.009.
87. Lee J, Ryoo N-k, Han H, Hong H K, Park J Y, Park S J, Kim Y-K, Sim C, Kim K, Woo S J. Anti-VEGF PolysiRNA polyplex for the treatment of choroidal neovascularization. Molecular pharmaceutics. 2016; 13(6):1988-95.
88. Apaolaza P, Del Pozo-Rodríguez A, Solinís M, Rodríguez J, Friedrich U, Torrecilla J, Weber B H, Rodríguez-Gascón A. Structural recovery of the retina in a retinoschisin-deficient mouse after gene replacement therapy by solid lipid nanoparticles. Biomaterials. 2016; 90:40-9.
89. Gan L, Wang J, Zhao Y, Chen D, Zhu C, Liu J, Gan Y. Hyaluronan-modified core-shell liponanoparticles targeting CD44-positive retinal pigment epithelium cells via intravitreal injection. Biomaterials. 2013; 34(24):5978-87.
90. D'Ambrosio D N, Clugston R D, Blaner W S. Vitamin A metabolism: an update. Nutrients. 2011; 3(1):63-103.
91. Shaw N S, Noy N. Interphotoreceptor retinoid-binding protein contains three retinoid binding sites. Exp Eye Res. 2001; 72(2):183-90.
92. Jin M, Li S, Nusinowitz S, Lloyd M, Hu J, Radu R A, Bok D, Travis G H. The role of interphotoreceptor retinoid-binding protein on the translocation of visual retinoids and function of cone photoreceptors. J Neurosci. 2009; 29(5):1486-95.
93. Sakurai E, Ozeki H, Kunou N, Ogura Y. Effect of particle size of polymeric nanospheres on intravitreal kinetics. Ophthalmic research. 2001; 33(1):31-6.
94. Leaderer D, Cashman S M, Kumar-Singh R. G-quartet oligonucleotide mediated delivery of proteins into photoreceptors and retinal pigment epithelium via intravitreal injection. Experimental Eye Research. 2016; 145:380-92. doi: https://doi.org/10.1016/j.exer.2016.02.009.
95. Du J, Sun Y, Li F-H, Du L-F, Duan Y-R. Enhanced delivery of biodegradable mPEG-PLGA-PLL nanoparticles loading Cy3-labelled PDGF-BB siRNA by UTMD to rat retina. Journal of biosciences. 2017; 42(2):299-309.
96. Huang D, Chen Y-S, Thakur S S, Rupenthal I D. Ultrasound-mediated nanoparticle delivery across ex vivo bovine retina after intravitreal injection. European Journal of Pharmaceutics and Biopharmaceutics. 2017; 119:125-36. doi: https://doi.org/10.1016/j.ejpb.2017.06.009.
97. Rajala A, Wang Y, Zhu Y, Ranjo-Bishop M, Ma J-X, Mao C, Rajala R V S. Nanoparticle-assisted targeted delivery of eye-specific genes to eyes significantly improves the vision of blind mice in vivo. Nano letters. 2014; 14(9):5257-63.
98. Johnson L N, Cashman S M, Kumar-Singh R. Cell-penetrating peptide for enhanced delivery of nucleic acids and drugs to ocular tissues including retina and cornea. Mol Ther. 2008; 16(1):107-14.
99. Read S P, Cashman S M, Kumar-Singh R. A poly (ethylene) glycolylated peptide for ocular delivery compacts DNA into nanoparticles for gene delivery to post-mitotic tissues in vivo. J Gene Med. 2010; 12(1):86-96.
100. Capowski E E, Samimi K, Mayerl S J, Phillips M J, Pinilla I, Howden S E, Saha J, Jansen A D, Edwards K L, Jager L D, Barlow K, Valiauga R, Erlichman Z, Hagstrom A, Sinha D, Sluch V M, Chamling X, Zack D J, Skala M C, Gamm D M. Reproducibility and staging of 3D human retinal organoids across multiple pluripotent stem cell lines. Development. 2019; 146(1). Epub 2018 Dec. 21. doi: 10.1242/dev.171686. PubMed PMID: 30567931; PMCID: PMC6340149.
101. Singh R, Shen W, Kuai D, Martin J M, Guo X, Smith M A, Perez E T, Phillips M J, Simonett J M, Wallace K A, Verhoeven A D, Capowski E E, Zhang X, Yin Y, Halbach P J, Fishman G A, Wright L S, Pattnaik B R, Gamm D M. iPS cell modeling of Best disease: insights into the pathophysiology of an inherited macular degeneration. Hum Mol Genet. 2013; 22(3):593-607. Epub 2012 Nov. 10. doi: 10.1093/hmg/dds469. PubMed PMID: 23139242; PMCID: PMC3542866.
102. Stoeckius M, Hafemeister C, Stephenson W, Houck-Loomis B, Chattopadhyay P K, Swerdlow H, Satija R, Smibert P. Simultaneous epitope and transcriptome measurement in single cells. Nature methods. 2017; 14(9):865-8. Epub 2017 Aug. 2. doi: 10.1038/nmeth.4380. PubMed PMID: 28759029; PMCID: PMC5669064.
103. Peterson V M, Zhang K X, Kumar N, Wong J, Li L, Wilson D C, Moore R, McClanahan T K, Sadekova S, Klappenbach J A. Multiplexed quantification of proteins and transcripts in single cells. Nat Biotechnol. 2017; 35(10):936-9. Epub 2017 Aug. 31. doi: 10.1038/nbt.3973. PubMed PMID: 28854175.
104. Pattnaik B R, Hughes B A. Regulation of Kir channels in bovine retinal pigment epithelial cells by phosphatidylinositol 4,5-bisphosphate. Am J Physiol Cell Physiol. 2009; 297(4):C1001-11. Epub 2009 Jul. 31. doi: 10.1152/ajpcell.00250.2009. PubMed PMID: 19641096; PMCID: PMC2770741.
105. Shahi P K, Hermans D, York N, Brar S, Borys K, Capowski E E, Welch E, Pillers D-A M, Gamm D M, Pattnaik B R. Read-through of LCA16 nonsense mutation therapy using iPS-RPE cells. Investigative Ophthalmology & Visual Science. 2016; 57(12):1103-.
106. Pittler S J, Baehr W. Identification of a nonsense mutation in the rod photoreceptor cGMP phosphodiesterase beta-subunit gene of the rd mouse. Proc Natl Acad Sci USA. 1991; 88(19):8322-6. Epub 1991 Oct. 1. doi: 10.1073/pnas.88.19.8322. PubMed PMID: 1656438; PMCID: PMC52500.
107. Farber D B, Flannery J G, Bowes-Rickman C. The rd mouse story: Seventy years of research on an animal model of inherited retinal degeneration. Progress in retinal and eye research. 1994; 13(1):31-64. doi: https://doi.org/10.1016/1350-9462(94)90004-3.
108. Nishiguchi K M, Carvalho L S, Rizzi M, Powell K, Holthaus S M, Azam S A, Duran Y, Ribeiro J, Luhmann U F, Bainbridge J W, Smith A J, Ali R R. Gene therapy restores vision in rd1 mice after removal of a confounding mutation in Gpr179. Nature communications. 2015; 6:6006. Epub 2015 Jan. 24. doi: 10.1038/ncomms7006. PubMed PMID: 25613321; PMCID: PMC4354202.
109. Mansergh F, Orton N C, Vessey J P, Lalonde M R, Stell W K, Tremblay F, Barnes S, Rancourt D E, Bech-Hansen N T. Mutation of the calcium channel gene Cacna1f disrupts calcium signaling, synaptic transmission and cellular organization in mouse retina. Hum Mol Genet. 2005; 14(20):3035-46. Epub 2005 Sep. 13. doi: 10.1093/hmg/ddi336. PubMed PMID: 16155113.
110. Waldner D M, Giraldo Sierra N C, Bonfield S, Nguyen L, Dimopoulos I S, Sauve Y, Stell W K, Bech-Hansen N T. Cone dystrophy and ectopic synaptogenesis in a Cacnalf loss of function model of congenital stationary night blindness (CSNB2A). Channels (Austin). 2018; 12(1):17-33. Epub 2017 Nov. 29. doi: 10.1080/19336950.2017.1401688. PubMed PMID: 29179637; PMCID: PMC5972796.
111. Pang J J, Chang B, Hawes N L, Hurd R E, Davisson M T, Li J, Noorwez S M, Malhotra R, McDowell J H, Kaushal S, Hauswirth W W, Nusinowitz S, Thompson D A, Heckenlively J R. Retinal degeneration 12 (rd12): a new, spontaneously arising mouse model for human Leber congenital amaurosis (LCA). Mol Vis. 2005; 11:152-62. Epub 2005 Mar. 15. PubMed PMID: 15765048.
112. Hart N S, Mountford J K, Voigt V, Fuller-Carter P, Barth M, Nerbonne J M, Hunt D M, Carvalho L S. The Role of the Voltage-Gated Potassium Channel Proteins Kv8.2 and Kv2.1 in Vision and Retinal Disease: Insights from the Study of Mouse Gene Knock-Out Mutations. eNeuro. 2019; 6(1). Epub 2019 Mar. 2. doi: 10.1523/ENEURO.0032-19.2019. PubMed PMID: 30820446; PMCID: PMC6393689.
113. He Q, Hsueh M, Zhang G, Joy D C, Leapman R D. Biological serial block face scanning electron microscopy at improved z-resolution based on Monte Carlo model. Sci Rep. 2018; 8(1):12985. Epub 2018 Aug. 30. doi: 10.1038/s41598-018-31231-w. PubMed PMID: 30154532; PMCID: PMC6113311.
114. Denk W, Horstmann H. Serial block-face scanning electron microscopy to reconstruct three-dimensional tissue nanostructure. PLOS Biol. 2004; 2(11):e329. Epub 2004 Oct. 30. doi: 10.1371/journal.pbio.0020329. PubMed PMID: 15514700; PMCID: PMC524270.
115. Mustafi D, Kikano S, Palczewski K. Serial block face-scanning electron microscopy: a method to study retinal degenerative phenotypes. Curr Protoc Mouse Biol. 2014; 4(4):197-204. Epub 2015 Jan. 27. doi: 10.1002/9780470942390.mo140169. PubMed PMID: 25621191; PMCID: PMC4303034.
116. Ohtsuka M, Sato M, Miura H, Takabayashi S, Matsuyama M, Koyano T, Arifin N, Nakamura S, Wada K, Gurumurthy C B. i-GONAD: a robust method for in situ germline genome engineering using CRISPR nucleases. Genome Biol. 2018; 19(1):25. Epub 2018 Feb. 28. doi: 10.1186/s13059-018-1400-x. PubMed PMID: 29482575; PMCID: PMC5828090.
117. Chan K, Hoon M, Pattnaik B R, Ver Hoeve J N, Wahlgren B, Gloe S, Williams J, Wetherbee B, Kiland J A, Vogel K R, Jansen E, Salomons G, Walters D, Roullet J B, Gibson K M, Mclellan G J. Vigabatrin-Induced Retinal Functional Alterations and Second-Order Neuron Plasticity in C57BL/6J Mice. Invest Ophthalmol Vis Sci. 2020; 61(2):17. Epub 2020 Feb. 14. doi: 10.1167/iovs.61.2.17. PubMed PMID: 32053727.
118. Mustafi D, Kevany B M, Genoud C, Okano K, Cideciyan A V, Sumaroka A, Roman A J, Jacobson S G, Engel A, Adams M D, Palczewski K. Defective photoreceptor phagocytosis in a mouse model of enhanced S-cone syndrome causes progressive retinal degeneration. FASEB J. 2011; 25(9):3157-76. Epub 2011 Jun. 11. doi: 10.1096/fj.11-186767. PubMed PMID: 21659555; PMCID: PMC3157681.
119. Morgans C W, Bayley P R, Oesch N W, Ren G, Akileswaran L, Taylor W R. Photoreceptor calcium channels: insight from night blindness. Vis Neurosci. 2005; 22(5):561-8. Epub 2005 Dec. 8. doi: 10.1017/S0952523805225038. PubMed PMID: 16332266.
120. Stone J, Johnston E. The topography of primate retina: a study of the human, bushbaby, and new- and old-world monkeys. J Comp Neurol. 1981; 196(2):205-23. Epub 1981 Feb. 20. doi: 10.1002/cne.901960204. PubMed PMID: 7217355.
121. Raghunathan V, Eaton J S, Christian B J, Morgan J T, Ver Hoeve J N, Yang C C, Gong H, Rasmussen C A, Miller P E, Russell P, Nork T M, Murphy C J. Biomechanical, ultrastructural, and electrophysiological characterization of the non-human primate experimental glaucoma model. Sci Rep. 2017; 7(1):14329. Epub 2017 Nov. 1. doi: 10.1038/s41598-017-14720-2. PubMed PMID: 29085025; PMCID: PMC5662689.
122. Jacobson S G, Boye S L, Aleman T S, Conlon T J, Zeiss C J, Roman A J, Cideciyan A V, Schwartz S B, Komaromy A M, Doobrajh M, Cheung A Y, Sumaroka A, Pearce-Kelling S E, Aguirre G D, Kaushal S, Maguire A M, Flotte T R, Hauswirth W W. Safety in nonhuman primates of ocular AAV2-RPE65, a candidate treatment for blindness in Leber congenital amaurosis. Hum Gene Ther. 2006; 17(8):845-58. Epub 2006 Sep. 1. doi: 10.1089/hum.2006.17.845. PubMed PMID: 16942444.
123. Ye G J, Budzynski E, Sonnentag P, Nork T M, Miller P E, Sharma A K, Ver Hoeve J N, Smith L M, Arndt T, Calcedo R, Gaskin C, Robinson P M, Knop D R, Hauswirth W W, Chulay J D. Safety and Biodistribution Evaluation in Cynomolgus Macaques of rAAV2tYF-PR1.7-hCNGB3, a Recombinant AAV Vector for Treatment of Achromatopsia. Human gene therapy Clinical development. 2016; 27(1):37-48. Epub 2016 Mar. 24. doi: 10.1089/humc.2015.164. PubMed PMID: 27003753; PMCID: PMC4851175.
124. Kim C B, Ver Hoeve J N, Kaufman P L, Nork T M. Interspecies and gender differences in multifocal electroretinograms of cynomolgus and rhesus macaques. Doc Ophthalmol. 2004; 109(1):73-86. Epub 2005 Jan. 29. doi: 10.1007/s10633-004-2630-7. PubMed PMID: 15675202.
125. Kim C B, VerHoeve J N, Kaufman P L, Nork T M. Effects of reference electrode location on monopolar-derived multifocal electroretinograms in cynomolgus monkeys. Doc Ophthalmol. 2005; 111(2):113-25. Epub 2006 Mar. 4. doi: 10.1007/s10633-005-4781-6. PubMed PMID: 16514493.
126. Nork T M, Murphy C J, Kim C B, Ver Hoeve J N, Rasmussen C A, Miller P E, Wabers H D, Neider M W, Dubielzig R R, McCulloh R J, Christian B J. Functional and anatomic consequences of subretinal dosing in the cynomolgus macaque. Archives of ophthalmology (Chicago, Ill: 1960). 2012; 130(1):65-75. Epub 2011 Sep. 14. doi: 10.1001/archophthalmol.2011.295. PubMed PMID: 21911651; PMCID: PMC3254795.
127. Bantseev V, Miller P E, Nork T M, Rasmussen C A, Mckenzie A, Christian B J, Booler H, Thackaberry E A. Determination of a No Observable Effect Level for Endotoxin Following a

Single Intravitreal Administration to Cynomolgus Monkeys. J Ocul Pharmacol Ther. 2019; 35(4):245-53. Epub 2019 Apr. 10. doi: 10.1089/jop.2018.0149. PubMed PMID: 30964386.
128. Boye S E, Alexander J J, Boye S L, Witherspoon C D, Sandefer K J, Conlon T J, Erger K, Sun J, Ryals R, Chiodo V A, Clark M E, Girkin C A, Hauswirth W W, Gamlin P D. The human rhodopsin kinase promoter in an AAV5 vector confers rod- and cone-specific expression in the primate retina. Hum Gene Ther. 2012; 23(10):1101-15. Epub 2012 Aug. 1. doi: 10.1089/hum.2012.125. PubMed PMID: 22845794; PMCID: PMC3472519.
129. Meyer J S, Howden S E, Wallace K A, Verhoeven A D, Wright L S, Capowski E E, Pinilla I, Martin J M, Tian S, Stewart R, Pattnaik B, Thomson J, Gamm D M. Optic Vesicle-like Structures Derived from Human Pluripotent Stem Cells Facilitate a Customized Approach to Retinal Disease Treatment. Stem Cells. 2011. Epub 2011 Jun. 17. doi: 10.1002/stem.674. PubMed PMID: 21678528.
130. Phillips M J, Jiang P, Howden S, Barney P, Min J, York N W, Chu L F, Capowski E E, Cash A, Jain S, Barlow K, Tabassum T, Stewart R, Pattnaik B R, Thomson J A, Gamm D M. A Novel Approach to Single Cell RNA-Sequence Analysis Facilitates In Silico Gene Reporting of Human Pluripotent Stem Cell-Derived Retinal Cell Types. Stem Cells. 2018; 36(3):313-24. Epub 2017 Dec. 13. doi: 10.1002/stem.2755. PubMed PMID: 29230913; PMCID: PMC5823737.
131. Chen G, Abdeen A A, Wang Y, Shahi P K, Robertson S, Xie R, Suzuki M, Pattnaik B R, Saha K, Gong S. A biodegradable nanocapsule delivers a Cas9 ribonucleoprotein complex for in vivo genome editing. Nat Nanotechnol. 2019; 14(10):974-80. Epub 2019 Sep. 11. doi: 10.1038/s41565-019-0539-2. PubMed PMID: 31501532; PMCID: PMC6778035.
132. Jung Y H, Phillips M J, Lee J, Xie R, Ludwig A L, Chen G, Zheng Q, Kim T J, Zhang H, Barney P, Min J, Barlow K, Gong S, Gamm D M, Ma Z. 3D Microstructured Scaffolds to Support Photoreceptor Polarization and Maturation. Adv Mater. 2018; 30(39):e1803550. Epub 2018 Aug. 16. doi: 10.1002/adma.201803550. PubMed PMID: 30109736.

Although the foregoing specification and examples fully disclose and enable the present invention, they are not intended to limit the scope of the invention, which is defined by the claims appended hereto.

All publications, patents and patent applications are incorporated herein by reference. While in the foregoing specification this invention has been described in relation to certain embodiments thereof, and many details have been set forth for purposes of illustration, it will be apparent to those skilled in the art that the invention is susceptible to additional embodiments and that certain of the details described herein may be varied considerably without departing from the basic principles of the invention.

The use of the terms “a” and “an” and “the” and similar referents in the context of describing the invention are to be construed to cover both the singular and the plural, unless otherwise indicated herein or clearly contradicted by context. The terms “comprising,” “having,” “including,” and “containing” are to be construed as open-ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Embodiments of this invention are described herein, including the best mode known to the inventors for carrying out the invention. Variations of those embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A method of reading through one or more premature termination codons (PTCs) in cells of the eye, comprising delivering one or more of the tRNAs, one or more of the oligonucleotide(s) encoding the tRNA(s), one or more of the vector(s) encoding the tRNA(s), one or more of the expression cassette(s) encoding the tRNA(s), or one or more of the composition(s) comprising any of the foregoing provided herein to cells of the eye in an amount effective to read through the one or more PTCs.

2. A method of restoring protein function in cells of the eye, comprising delivering one or more of the tRNAs, one or more of the oligonucleotide(s) encoding the tRNA(s), one or more of the vector(s) encoding the tRNA(s), one or more of the expression cassette(s) encoding the tRNA(s), or one or more of the composition(s) comprising any of the foregoing provided herein to cells of the eye in an amount effective to restore protein function in the cells.

3. A method of restoring vision in a subject, comprising administering one or more of the tRNAs, one or more of the oligonucleotide(s) encoding the tRNA(s), one or more of the vector(s) encoding the tRNA(s), one or more of the expression cassette(s) encoding the tRNA(s), or one or more of the composition(s) comprising any of the foregoing provided herein to cells of the eye in an amount effective to restore vision in the subject.

4. The method of claim 3, wherein the administration is by subretinal injection.

5. The method of claim 3, wherein the administration is by intravitreal injection.

6. The method of any one of claims 3-5, wherein the subject is a human.

7. The method of any one of the preceding claims, wherein the cells are photoreceptor cells.

8. The method of any one of claims 1-6, wherein the cells are retinal pigment epithelial cells.

9. The method of any one of the preceding claims wherein the cells are iPSCs.

10. The method of any one of the preceding claims, wherein the one or more oligonucleotide(s) or one or more expression cassette(s) are DNA, such as cDNA.

11. The method of any one of the preceding claims, wherein the one or more oligonucleotide(s) or one or more expression cassette(s) are in the form of a plasmid.

12. The method of any one of the preceding claims, wherein the one or more of the oligonucleotide(s) or one or more of the expression cassette(s) are in the form of a minicircle.

13. The method of any one of claims 1-9, wherein the vector is a viral vector.

14. The method of claim 13, wherein the viral vector is an AAV vector.

15. The method of claim 14, wherein the AAV vector is an AAV2, AAV5, AAV9, AAV2/9, AAV7m8 or AAV2.7m8 vector.

16. The method of any one of the preceding claims, wherein at least two or at least three of any of the tRNAs provided herein are delivered or administered.

17. The method of any one of claims 1-15, wherein the at least two or at least three of tRNAs provided herein are encoded by the one or more oligonucleotide(s), one or more vector(s), one or more expression cassette(s), or are comprised in one or more of the composition(s).

18. The method of any one of the preceding claims, wherein the one or more of the tRNAs are ArgtRNA(s), TrptRNA(s) and/or GlutRNA(s).

19. The method of any one of the preceding claims, wherein the one or more of the tRNAs, one or more of the oligonucleotide(s) encoding the tRNA(s), one or more of the vector(s) encoding the tRNA(s), one or more of the expression cassette(s) encoding the tRNA(s), or one or more of the composition(s) comprising any of the foregoing provided herein are comprised in a delivery vehicle.

20. The method of claim 19, wherein the delivery vehicle is a particle.

21. The method of claim 20, wherein the particle is a lipid-based particle, such as a liposome.

22. The method of claim 20, wherein the particle is a polymer-based particle.

23. The method of claim 20, wherein the particle is a silica nanoparticle (SNP).

24. The method of claim 23, wherein the SNP is delivered or administered as a population of SNPs.

25. The method of claim 24, wherein the SNPs are conjugated to HA.

26. The method of claim 25, wherein the molecular weight of the HA is in the range of 10 kDa-1MDa, such as any one specific molecular weights provided herein.

27. The method of any one of claims 23-26, wherein the SNP(s) has surface amine groups, such as APTES amine groups.

28. The method of claim 27, wherein the molar ratio of APTES to total silica of the SNP(s) is in the range of 10-30%, such as any one specific molar ratio provided herein.

29. The method of claim 27 or 28, wherein the molar ration of HA saccharide unit to APTES of the SNP(s) is 5-15, such as any one specific molar ratio provided herein.

30. The method of any one of claims 23-29, wherein the SNP(s) are conjugated to a targeting ligand or cell penetrating peptide (CPP), such as any one of the targeting ligands or CPPs provided herein.

31. The method of claim 30, wherein the molar ratio of targeting ligand or CPP to HA is 10-30%, such as any one specific molar ratio provided herein.

32. The method of any one of claims 25-31, wherein the substitution degree of HA to targeting ligand or CPP conjugate is 1-40%, such as any one specific substitution degree provided herein.

33. The method of any one of claims 23-32, wherein the SNP(s) are conjugated to PEG.

34. The method of claim 33, wherein the PEG links the targeting ligand or CPP to the SNP(s).

35. The method of claim 33 or 34, wherein the weight ratio of PEG/SNP is 1/10-3/10, such as any specific weight ratio provided herein.

36. The method of any one of claims 33-35, wherein the targeting ligand or CPP ratio to PEG is 1-60, such as any one specific ratio provided herein.

37. The method of any one of claim 30 or 31, wherein the targeting ligand or CPP is conjugated to the SNP(s) with a spacer, such as OEG.

38. The method of claim 37, wherein the OEG has a molecular weight of 200 Da-1 kDa, such as any one specific molecular weight provided herein.

39. The method of any one of claims 23-38, wherein the average dimension of the SNP(s) is between 40-60 nm, such as any one specific average dimension provided herein.

40. A composition comprising the tRNA(s), oligonucleotide(s), vector(s), expression cassette(s) or composition(s) of any one of the preceding claims.

41. A composition comprising the delivery vehicle or population of delivery vehicles, such as the particles, of any one of claims 19-40.

42. The composition of claim 40 or 41, further comprising a pharmaceutically acceptable carrier.

43. A cell comprising the tRNA(s), oligonucleotide(s), vector(s), expression cassette(s) or composition(s) of any one of the preceding claims.

44. The cell of claim 43, wherein the cell is a photoreceptor cell.

45. The cell of claim 43, wherein the cell is a retinal pigment epithelial cell.

46. The cell of any one of claims 43-45, wherein the cell is an iPSC.

47. The cell of any one of claims 43-26 that is of a mouse model, such as provided herein.

48. The cell of claim 47, wherein the mouse model is a Kcnv2 and Cacna1f mutant mouse, such as provided herein.

49. A Kcnv2 mutant mouse.

50. A Cacna1f mutant mouse.
